(19) United States
(12) Patent Application Publication
(10) Pub. No.: US 2003/0229113 A1
(43) Pub. Date: Dec. 11, 2003

(54) Keratinocyte growth inhibitors and hydroxamic acid derivatives

(76) Inventors: Koji Hashimoto, Ehime (JP); Shigeki Higashiyama, Osaka (JP); Kohichiro Yoshino, Osaka (JP); Kazuya Yoshiizumi, Osaka (JP); Minoru Yamamoto, Osaka (JP)

Correspondence Address: William M Blackstone, Akzo Nobel Patent Department, Intervet Inc, 405 State Street, Millsboro, DE, 19966, US

(21) Appl. No.: 10/239,675

(22) PCT Filed: Mar. 22, 2001
(86) PCT No.: PCT/JP01/02251

(51) Int. Cl. A61K 31/4745; A61K 31/47

(52) U.S. Cl. 514/291; 514/303; 514/310

(57) ABSTRACT

This invention relates to a keratinocyte-proliferation inhibitor comprising as active ingredient a compound having an activity of inhibiting the solubilization of heparin-binding EGF-like growth factor bound to cell membranes and a compound of the formula (I);

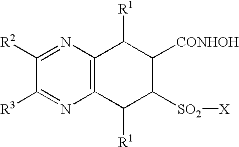

or pharmaceutically acceptable salt thereof, wherein R¹, R², R³ are hydrogen atom or alkyl and X is substituted benzene or the like.

### TECHNICAL FIELD

This invention relates to an inhibitor of proliferation of keratinocytes and hydroxamic acid derivatives which are an active ingredient of the inhibitor. More specifically, it relates to the inhibitor comprising as active ingredient a compound having an activity of inhibiting the solubilization of heparin-binding EGF-like growth factor (hereinafter referred to as “HB-EGF”) bound to cell membranes. Furthermore, it relates to novel hydroxamic acid derivatives having an inhibitory activity on the solubilizing enzyme of HB-EGF.

### BACKGROUND ART

Keratinocytes occupying major portions of epidermal cell layers of skin play an important role in the barrier function of the skin. Excessive proliferation of keratinocytes is observed in dermatitis such as psoriasis, photogenic keratosis, ichthyosis, excrescence, seborrheic dermatitis or atopic dermatitis and skin diseases such as skin cancer.

As a keratinocyte proliferation factor, epithelium growth factor (EGF), transforming growth factor-α (TGF-α), anphiregulin (AR), HB-EGF, fibroblast growth factor-1 (FGF-1), FGF-2, FGF-7, hepatocyte growth factor, etc., have been reported (J. Invest. Dermatol., 115: 715-721 (1988)). It is known that factors of EGF family such as EGF, TGF-α, AR and HB-EGF are synthesized as membrane-bound proteins which are broken into a soluble form by the action of metal enzymes (hereinafter referred to as “solubilizing enzymes”) and that both membrane-bound and soluble forms bind to EGF receptor. (EMBO J., 17:7260-7272 (1998); Nature, 402:884-888 (1999); Proc. Natl. Acad. Sci. USA, 25:6235-6240 (1990)).

It is not necessarily clear, however, how these factors are involved in the above-mentioned keratinocyte-proliferative diseases. It is also unknown which form namely the soluble form or membrane-binding form of EGF-family factors is mainly involved in diseases associated with aberrant proliferation of keratinocytes.

### DISCLOSURE OF INVENTION

It has been desired to develop a new drug for the prevention or treatment of the proliferation of keratinocytes. The object of the present invention is to provide a new agent suppressing the proliferation of keratinocytes based on the new mechanism, and furthermore, new compounds having an inhibitory activity on the solubilizing enzyme of HB-EGF.

The inventors studied which growth factor or factors were increasing in the aberrant proliferation site of a patient with psoriasis which is typical of diseases associated with aberrant proliferation of keratinocytes by immunostaining the epithelium layer of the patient with antibodies against respective growth factors. As a result, a remarkable increase of HB-EGF was found in the epithelium layer of the patient with psoriasis.

Next, the involvement of HB-EGF in the proliferation of keratinocytes was demonstrated using in vivo wound model of Tsuboi et al. (J. Dermatol., 19:673-675 (1992)). To this end, human keratinocytes were cultured on a collagen-coated petri dish. Then a portion of the culture was cut out to make a recess and the culturing was continued whereupon the recess was filled again with proliferated keratinocytes by migration. When anti-HB-EGF neutralizing antibody or anti-EGF receptor neutralizing antibody was present, remarkable suppression of the proliferation and migration of keratinocytes was observed indicating the critical importance of HB-EGF in the proliferation and migration of keratinocytes.

In the next experiment, the breakage and thus production of soluble form of HB-EGF was prevented in order to determine which form, namely membrane-binding form or soluble form of HB-EGF played an important role in the proliferation and migration of keratinocytes in the above wound model. As a result, the proliferation and migration of keratinocytes were remarkably suppressed as well when a portion of cultured keratinocytes was cut out and the culturing was continued in the presence of an inhibitor of solubilizing enzyme in place of the above antibodies.

In a further experiment, the inhibitory effect of inhibitor of solubilizing enzyme on the aberrant proliferation of keratinocytes in mouse model (in vivo) was confirmed. Namely, the inhibitor of solubilizing enzyme remarkably suppressed epidermal hyperplasia caused by aberrant proliferation of keratinocytes induced by the application of a phorbol ester on the skin of mouse (see, Test Example 2 below). The inhibitor of solubilizing enzyme also remarkably suppressed the proliferation and migration of keratinocytes in the skin wound model of mouse used as a model of psoriasis (see, Test Example 3 below).

From the foregoing results, the inventors have found that the soluble form of HB-EGF plays a very important role in the proliferation of keratinocytes, and confirmed that the proliferation of keratinocytes may be suppressed by preventing the production of the said soluble HB-EGF. Furthermore, they have found that some hydroxamic acids have an inhibitory activity on the solubilizing enzyme of HB-EGF and that they are useful as an inhibitor of proliferation of keratinocytes. The invention was completed in this way.

### BEST MODE FOR CARRYING OUT THE INVENTION

The proliferation inhibitor according to the present invention comprises as active ingredient a compound capable of inhibiting the solubilizing enzyme of HB-EGF. As the active ingredient, any compound will be used if it shows inhibitory activity on solubilizing enzyme in human, and there may be used, for example, but not limited thereto, a compound of the formula (I):

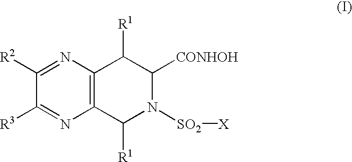

or pharmaceutically acceptable salt thereof, wherein

R¹ is hydrogen atom or methyl;

R² and R³ may be the same or different, and are hydrogen atom or C₁-C₄ alkyl;

X is selected from the following (A), (B), (C) and (D) groups

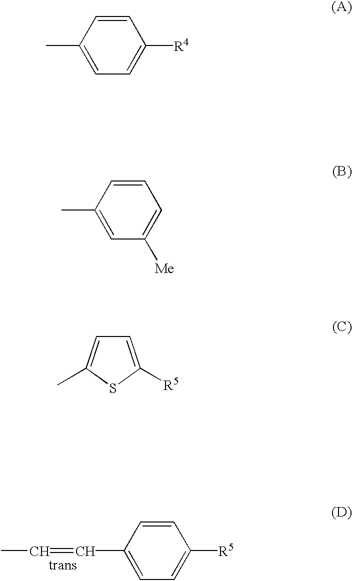

wherein

R⁴ is hydrogen atom, halogen atom, hydroxyl, amino, C₁-C₈ alkyl, vinyl, ethynyl, phenyl, C₁-C₈ alkylthio, phenoxy, 4-aminophenoxy, heteroaryloxy, C₁-C₈ acyloxy, C₁-C₈ acyl or C₁-C₈ alkoxy, wherein the said C₁-C₈ alkoxy may be substituted by 2-propinyl, 2-butinyl, phenyl, halogen atom, C₁-C₄ alkoxyl, C₁-C₄ alkylthio, C₁-C₄ alkylsulfonyl, cyano, C₁-C₄ alkoxylcarbonyl, C₁-C₄ acyloxy or hydroxyl;

R⁵ is hydrogen atom or C₁-C₈ alkyl which may be substituted by C₁-C₄ alkoxy.

In this specification, “C₁-C₄ alkyl” refers to an alkyl which includes a straight chain, a branched chain, a cyclic moiety or combination thereof having 1 to 4 carbon atoms, and an alkyl such as methyl, ethyl, isopropyl or isobutyl is exemplified. In the same way, “C₁-C₈ alkyl” refers to an alkyl which includes a straight chain, a branched chain, a cyclic moiety or combination thereof having 1 to 8 carbon atoms, and an alkyl such as methyl, ethyl, isopropyl, isobutyl, cyclopentyl or cyclohexyl is exemplified.

“Heteroaryl” refers to 5- or 6-membered aromatic group having one or two heteroatoms selected from a group of a sulfur, oxygen or nitrogen atom, and a group such as a thiophen, furan, pyrrole, imidazole or pyridine ring is exemplified. “Halogen atom” refers to a fluorine, chlorine, bromine or iodine atom.

The compounds of the formula (I) have a plural number of stereoisomers and the present invention include all the stereoisomers and mixtures thereof.

Above all, stereochemistry of the carbon in a-position of —CONHOH group is important, and it profoundly affects the inhibitory activity against a target enzyme. Usually R-enantiomer is preferable, if it is indicated by R—S method.

The pharmaceutically acceptable salt includes, for example, a salt with an inorganic base, such as sodium salt, potassium salt, calcium salt, or a salt with an organic base, such as arginine salt, lysine salt.

The processes of the production of the compounds (I) and their synthetic intermediate are explained below. Each abbreviation used in the formula means as follows, without being indicated specifically.

Bzl: benzyl group

Et: ethyl group

Me: methyl group

t-Bu or Bu-t: tert-butyl group

Z: benzyloxycarbonyl group

Ac: acetyl group

Boc tert-butyloxycarbonyl group

1. Preparation of Compounds (I)

The compounds of the present invention are prepared by Method-A or Method-B as shown in the following scheme 1 or scheme 2 respectively. In a case that the X-moiety of the compound (I) includes functional groups, which are easily reduced such as vinyl, ethynyl, cyano or halogen atom, or which may poison catalysts of a reductive reaction like thiophen ring, preparation by Method-B is preferable. On the contrary, Method-A is preferable in a case that the X-moiety has substituents of amino or hydroxyl group, but Method-B is also possible if these groups are chemically protected according to a conventional method.

=Method-A(scheme 1)=

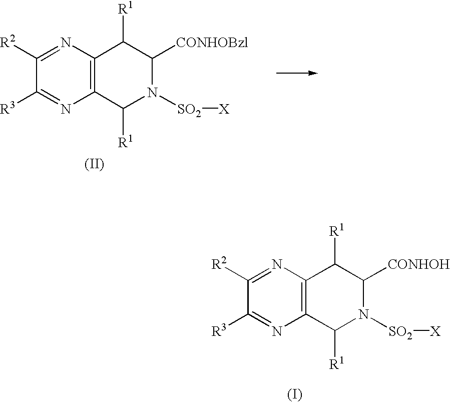

The compound (I) is prepared by hydrogenolysis of the compound (II), which is carried out in a lower alcohol such as methanol or ethanol, if necessary, by adding water, hydrochloric acid, acetic acid, N,N-dimethylformamide(hereinafter, abbreviated as “DMF”), 1,4-dioxane and the like, in the presence of a catalyst such as palladium-carbon(hereinafter, abbreviated as “Pd—C”) under hydrogen atmosphere or hydrogen pressure, at a temperature of room temperature to 60° C.

Particularly in the case of preparing the compound (I) which has an amino substituent at the X-moiety, the desired compound is advantageously obtained by Method-A using the corresponding nitro-substituted compound as a precursor. As shown below, the desired amino compound is prepared from the corresponding nitro-substituted compound by Method-A, in which deprotection of Bzl group at the hydroxamic acid moiety and reduction of the said nitro group proceed simultaneously (See, example 6).

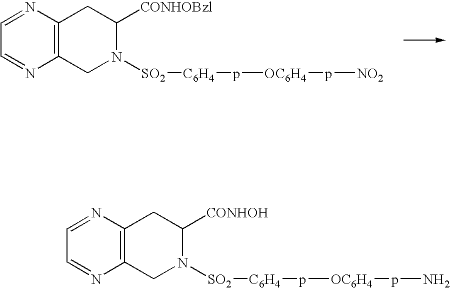

According to Method-B, the compounds of the present invention are prepared from compounds (III) (scheme 2).

=Method-B(scheme 2)=

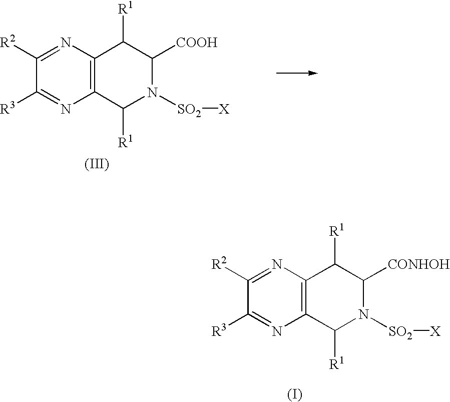

More specifically, each of the following method is available.

(1) After the compound (III) is converted into corresponding acid chloride in a solvent such as dichloromethane, by adding 1.0-5.0 mole of oxalyl chloride per 1.0 mole of the compound (III) and stirring for 15 minutes to 6 hours at a temperature of 0° C. to room temperature, the compound (I) is prepared by adding hydroxylamine to the reaction mixture and stirring at a temperature of 0° C. to room temperature for 0.5 to 6 hours.

(2) The compound (I) is prepared by condensing the compound (III) with hydroxylamine hydrochloride in an aprotic solvent such as DMF, tetrahydrofuran (hereinafter, abbreviated as “THF”) or dichloromethane using a condensing agent and an additive which are conventionally used in peptide synthesis, like 1-ethyl-3-(3-dimethylaminopropyl)carbodiimide hydrochloride (hereinafter, abbreviated as “WSC”) and 1-hydroxybenzotriazole hydrate (hereinafter, abbreviated as “HOBt”) for example; also it is prepared by condensing the compound (III) with a hydroxylamine derivative which was protected by a protective group, like O-(tert-butyldimethylsilyl)hydroxylamine for example, and by treating the product with an acid. The condensing reaction is usually carried out at a temperature of 0° C. to room temperature for 2 to 48 hours, and the molar ratio of the reactants are 1.0-7.0 mole of hydroxylamine hydrochloride or the protected hydroxylamine derivative, 1.0-5.0 mole of the condensing agent and 1.0-5.0 mole of the additive per 1.0 mole of the compound (III).

(3) The preparation of compound (I) is also possible through aminolysis of methyl or ethyl esters of compound (III) using a water solution of hydroxylamine in a solvent such as dimethoxyethane or THF. Furthermore, the method of Fieser (Fieser, Reagents for Organic Synthesis, John Willy and Sons, New York, Vol.1, pp.478-479) is also applicable to prepare the compound (I).

2. Preparation of compounds (II)

The precursor compound (II) shown in the scheme 1 is prepared by the steps as shown in the following scheme 3.

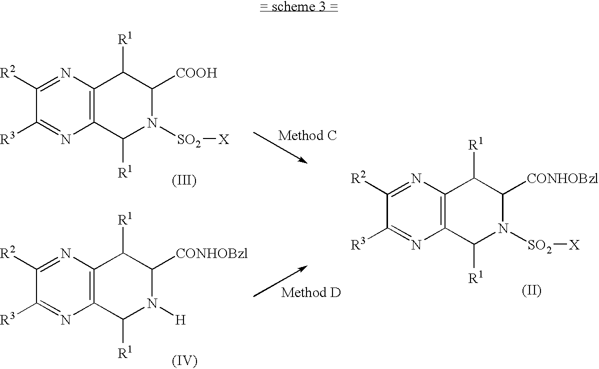

According to Method-C, the compound (II) is prepared by condensing the compound (III) with O-benzylhydroxylamine hydrochloride in an aprotic solvent such as DMF, THF or dichloromethane using a condensing agent (e.g., WSC) and an additive (e.g., HOBt) which are conventionally used in peptide synthesis. The reaction is usually carried out at a temperature of 0° C. to room temperature for 2-24 hours, and the molar ratio of the reactants are 1.0-2.5 mole of O-benzylhydroxylamine hydrochloride, 1.0-2.5 mole of the condensing agent and 1.0-2.5 mole of the additive per 1.0 mole of the compound (III).

According to Method-D, the compound (II) is prepared by reacting the compound (IV) with the corresponding sulfonyl chloride as shown below.

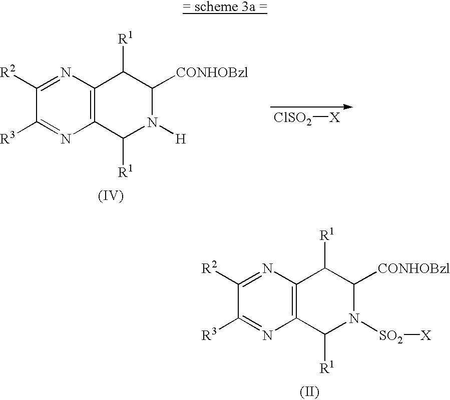

The compound (IV) is reacted with the corresponding sulfonyl chloride in dichloromethane or a mixture of water and 1,4-dioxane, and in the presence of a base such as 4-dimethylaminopyridine (hereinafter, abbreviated as “DMAP”) or triethylamine to give the compound (II). The molar ratio of the reactants are 2.0-3.0 mole of the base and 1.0-2.0 mole of the sulfonyl chloride per 1.0 mole of the compound (IV) and the reaction is carried out for 1.0-18 hours at a temperature of 0° C. to room temperature.

3. Preparation of compounds (III)

The precursor compound (III) shown in the above scheme 2 is prepared by Method E or Method F as shown in the following scheme 4 or scheme 5 respectively.

Method E is a process of preparing the compound (III) from a ester derivative (Va), (Vb) or (Vc) as shown in scheme 4.

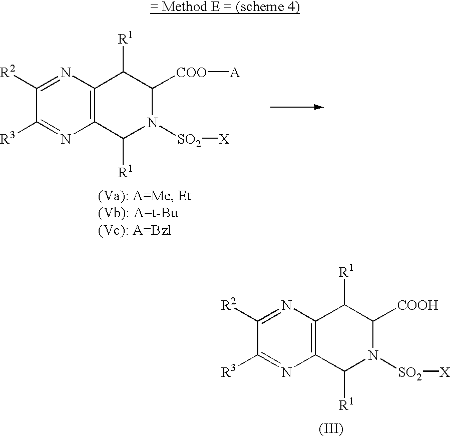

(1) The compound (III) is prepared by alkaline hydrolysis of the compound (Va), in which A is defined as a methyl or ethyl group. Practically, the compound (Va) is treated with an alkali such as sodium hydroxide in a mixture of water and 1,4-dioxane with stirring at a temperature of 0° C. to room temperature for 0.5-5 hours to give the compound (III). The molar ratio of the reactant is 1.0-5.0 mole of sodium hydroxide per 1.0 mole of the compound (Va). If the compound (III) has a functional group which is hydrolyzed easily by alkali, like an acyloxy group, the following methods of (2) or (3) are preferrable.

(2) The compound (III) is also prepared by acidlysis of the compound (Vb), in which A is defined as a tert-butyl group. Practically, the compound (Vb) is treated with an acid such as trifluoroacetic acid or formic acid with stirring at a temperature of 0° C. to room temperature for 1-6 hours.

(3) The compound (III) is also prepared by hydrogenolysis of the compound (Vc), in which A is defined as a benzyl group. In the reaction the compound (Vc) is subjected to hydrogenolysis in a lower alcohol such as methanol or ethanol, if necessary adding thereto water, acetic acid, DMF or 1,4-dioxane in the presence of a catalyst such as Pd—C in a hydrogen atmosphere or under a hydrogen pressure at a temperature of room temperature to 60° C.

Method F is a process of preparing the compound (III) by reacting the carboxylic acid derivative (VI) with sulfonyl chloride as shown in scheme 5.

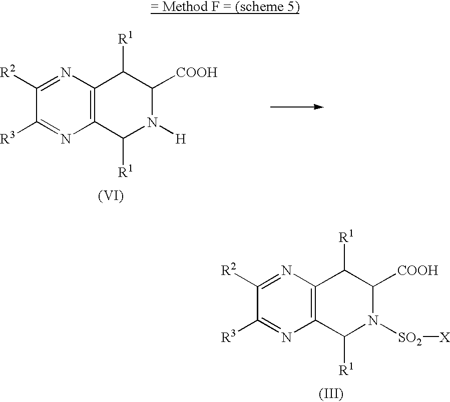

The compound (III) is prepared, for example, by reacting the derivative (VI) with the corresponding sulfonyl chloride in a mixture of 1,4-dioxane and water in the presence of a base such as sodium hydroxide.

4. Other Synthetic Intermediates

(1) Preparation of the Compound (V)

The compound (V) is prepared according to scheme 6 shown below.

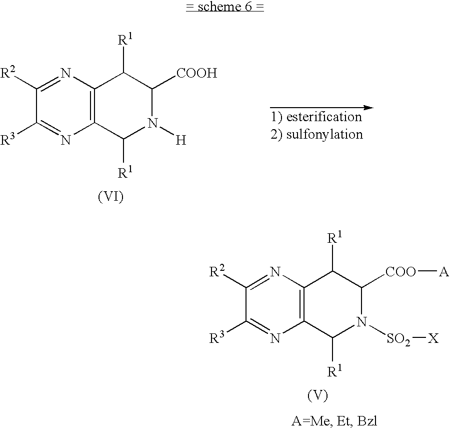

Methyl, ethyl or benzyl ester is prepared by a conventional method of esterification of amino acids. t-Butyl ester is prepared through benzyloxycarbonyl derivatives ((VI-2) and (VI-3)) as shown in scheme 7.

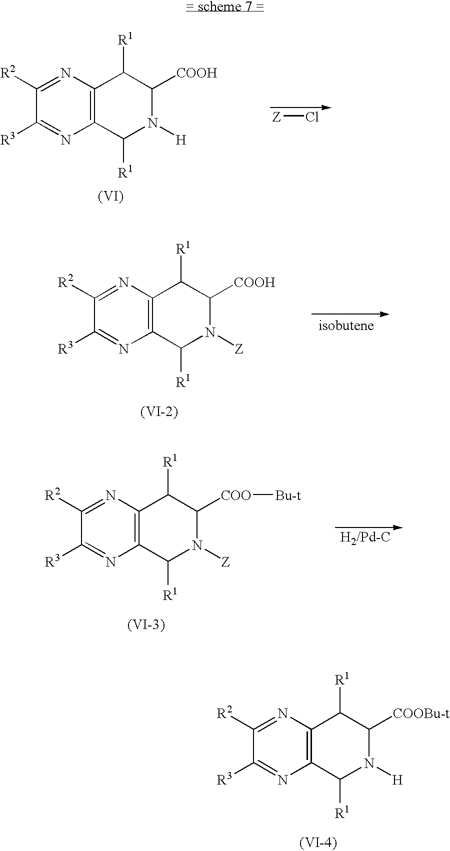

That is, the compound (VI-2) is prepared by reacting the compound (VI) with benzyloxycarbonylchloride (Z-Cl) in a mixture of water and 1,4-dioxane in the presence of alkali such as sodium hydroxide or sodium carbonate at a temperature of 0° C. to room temperature for 1-24 hours. The molar ratio of the reactants are 2.0-3.0 mole of sodium hydroxide and 1.0-2.0 mole of Z-Cl per 1.0 mole of the compound (VI).

Next, the tert-butyl ester (VI-3) is prepared by reacting the compound (VI-2) with an excess amount of isobutene in a solvent such as dichloromethane in the presence of an acid catalyst such as sulfuric acid at −40° C. to room temperature for 5 hours to 7 days.

Next, the tert-butyl ester (VI-3) is subjected to hydrogenolysis in a lower alcohol such as methanol or ethanol, if necessary adding thereto water, hydrochloric acid, acetic acid or DMF in the presence of a catalyst such as Pd—C in a hydrogen atmosphere or under a hydrogen pressure at a temperature of room temperature to 60° C. to give the compound (VI-4).

The compound (V) is obtained through sulfonylation of the esters of the compound (VI) according to the method D described above.

In a case that synthesis of the corresponding sulfonyl chloride is difficult, the objective compound is often prepared by building of the Xa moiety after an introduction of the benzylsulfonyl group. As an example, scheme 6a preparing the compound (V-3) is shown below, in which the p-ethynylphenylsulfonyl group is introduced by a method of substituting the ethynyl group after benzensulfonylation of the precursor compound.

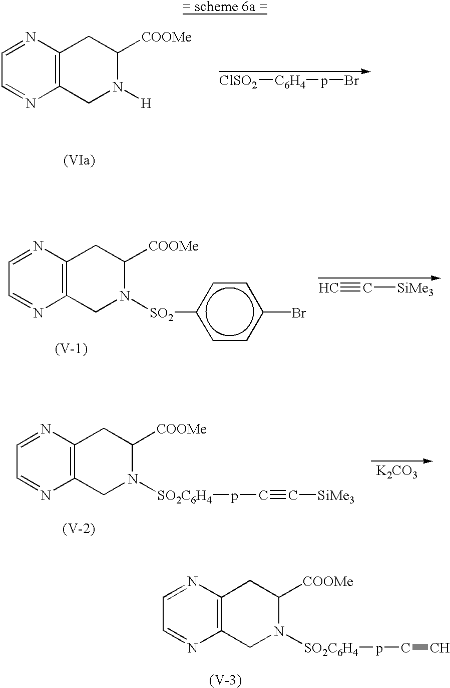

That is, the methyl ester (VIa) is reacted with 4-bromobenzensulfonyl chloride in a solvent such as DMF in the presence of a base such as dimethylaminopyridine or triethylamine to give the compound (V-1). The molar ratios of the reactants are 2.0-3.0 mole of the base and 1.0-3.0 mole of 4-bromobenzensulfonylchloride per 1.0 mole of the methyl ester (VIa).

The compound (V-1) is then reacted with trimethylsilylacetylene with stirring in a solvent such as triethylamine in the presence of a catalytic amount of dichlorobistriphenylphosphine palladium(II) and cuprous iodide at a temperature of −78° C. to refluxing temperature for 1-12 hours to give the compound (V-2). The molar ratio of the reactants is 1.0-3.0 mole of trimethylsilylacetylene per 1.0 mole of the compound (V-1).

The compound (V-2) is then stirred in methanol in the presence of sodium carbonate at a temperature of 0° C. to room temperature for 1-12 hours to give the compound (V-3). The molar ratio of the reactants is 1.0-3.0 mole of sodium carbonate per 1.0 mole of the compound (V-2).

The carboxylic acid derivative is alternatively prepared by subjecting the compound (V-2) to the deprotection of trimethylsilyl group and hydrolysis of the methyl ester group simultaneously by treating it with an alkali such as sodium hydroxide in a mixture of water and 1,4-dioxane

(2) Preparation of the Compound (VI)

The derivative (VI-8) is prepared from the compound (VI-5) by the steps as shown in the following scheme 8.

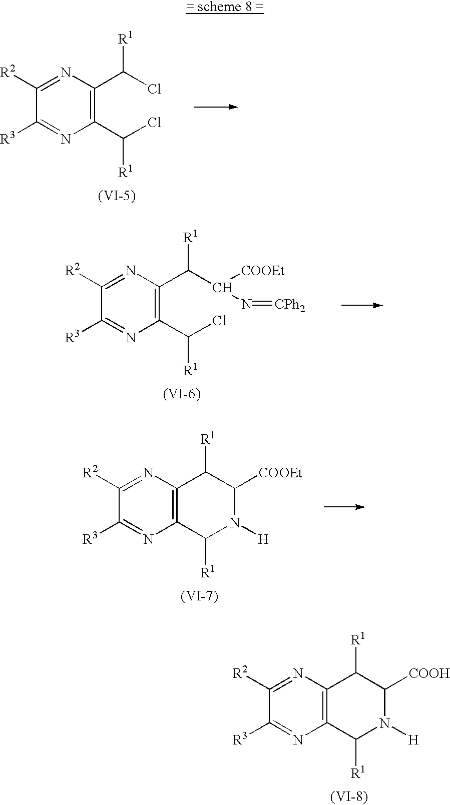

That is, the compound (VI-5) is stirred with N-(diphenylmethylene)glycine ethyl ester and an alkali such as sodium hydroxide in a solvent such as acetonitorile in the presence of a catalytic amount of benzyltriethylammonium chloride at a temperature of 0° C. to room temperature for 3-24 hours to give the compound (VI-6). The molar ratio of the reactants are 1.0-1.2 mole of N-(diphenylmethylene)glycine ethyl ester and 1.0-1.2 mole of sodium hydroxide per 1.0 mole of the compound (VI-5).

Next, the compound (VI-6) is stirred in a solvent such as diethylether in the presence of 1N hydrochloric acid at a temperature of 0° C. to room temperature for 1-12 hours, and then a base such as potassium carbonate was thereto added and the resulting mixture was stirred at a temperature of 0° C. to room temperature for 3-124 hours to give the compound (VI-7). The objective compound (VI-8) is obtained by alkaline hydrolysis of the compound (VI-7).

In case that R¹ is a hydrogen atom in the formula (VI-8), the corresponding carboxylic acid (VI-11) can be alternatively prepared from the bischloromethyl derivative (VI-9) as shown in the following scheme 9.

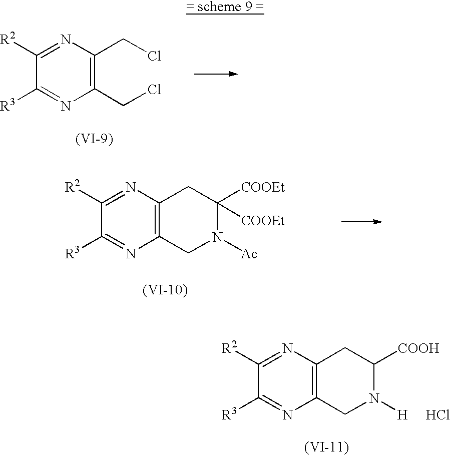

That is, the compound (VI-9) is reacted with diethyl acetamidomalonate with stirring in a solvent such as DMF or acetonitrile in the presence of a base such as sodium hydride, sodium ethoxide or cesium carbonate at a temperature of 0-70° C. for 1-24 hours to give the compound (VI-10). The molar ratios of the reactants are 2.0-3.0 mole of the base and 1.0-1.5 mole of diethyl acetamidomalonate per 1.0 mole of the compound (VI-9). After then, the compound (VI-11) is prepared by refluxing the compound (VI-10) in the presence of hydrochloric acid for 1-12 hours.

(3) Preparation of the Dichloro Derivative (VI-5)

The dichloro derivative (VI-5) which is a precursor for the preparation of the compound (VI-8) is prepared by the following method.

1) The compound (VI-5a) in which both of R² and R³ are hydrogen atoms in the formula (VI-5) can be prepared from the compound (VI-12) as shown in the following scheme 10.

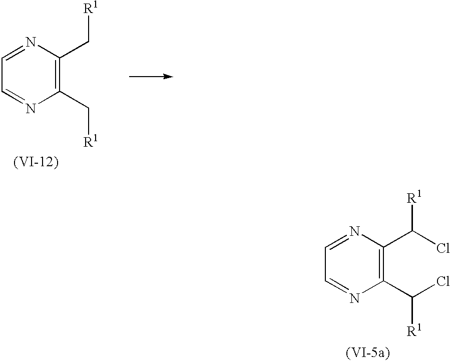

That is, the compound (VI-12) is treated with N-chlorosuccinimide with stirring in a solvent such as tetrachlorocarbon in the presence of a catalytic amount of radical initiator at a refluxing temperature for 2-24 hours to give the compound (VI-5a). Benzoyl peroxide is often used as a radical initiator and the molar ratio of the reactants is 2.0-3.0 mole of N-chlorosuccinimide per 1.0 mole of the compound (VI-12).

2) The compound (VI-5b) in which either of R² or R³ is C₁-C₄ alkyl in the formula (VI-5) can be prepared from the compound (VI-14) as shown in the following scheme 11.

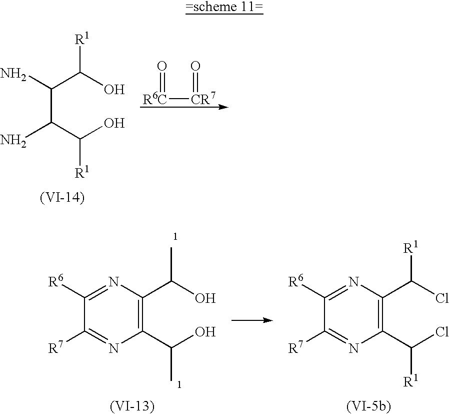

Wherein R⁶ and R⁷ mean a hydrogen atom or C₁-C₄ alkyl; but either one means C₁-C₄ alkyl.

That is, the compound (VI-13) is prepared by reacting the compound (VI-14) with 2,3-diamino-1,4-butanediol with stirring in a solvent such as methanol in the presence of a base such as potassium hydroxide at a room temperature for 1-3 hours, and then continuing the stirring of the reaction mixture while oxygen gas is blowing in at a room temperature for 1-48 hours. The molar ratio of the reactants are 1.0-2.0 mole of potassium hydroxide and 1.0-1.5 mole of 2,3-diamino-1,4-butanediol per 1.0 mole of the compound(VI-14).

Next, the compound (VI-13) is treated with phosphorus trichloride with stirring in a solvent such as DMF at a temperature of 0° C. to a room temperature for 1-12 hours to give the compound (VI-5b). The molar ratios of the reactants is 1.0-6.0 mole of the phosphorus trichloride per 1.0 mole of the compound(VI-13).

(4) Preparation of the Compound (IV)

The compound (IV) is prepared from the compound (VI) as shown in the following scheme 12.

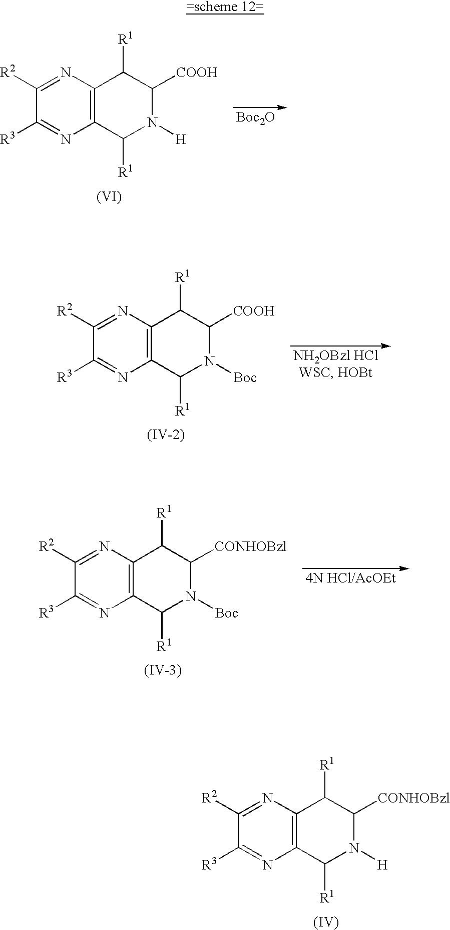

That is, the compound (VI) is reacted with di-tert-butyl dicarbonate in a mixture of 1,4-dioxane and water etc. in the presence of an alkali such as sodium hydroxide to give the compound (IV-2). The molar ratios of the reactants are 1.0-2.0 mole of sodium hydroxide and 1.0-2.0 mole of di-tert-butyl dicarbonate pre 1.0 mole of the compound (VI).

The compound (IV-3) is prepared by condensing the compound (IV-2) with O-benzylhydroxylamine hydrochloride by the method using WSC and HOBt described before.

The compound (IV) is prepared by treating the compound (IV-3) with a solution containing an excess amount of an acid such as 4N hydrogen chloride/ethyl acetate with stirring at a temperature of 0° C. to a room temperature for 0.5-6 hours.

(5) Preparation of the Optically Active Compound of (VI)

An optical resolution of the carboxylic acid derivative (IV-2) is possible using optically active amine as shown in the following scheme 13.

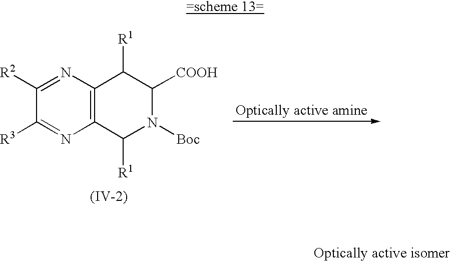

That is, a racemic mixture of the compound (IV-2) is treated with an equimolar amount of optically active amine such as (+)- or (−)-α-methylbenzylamine in a solvent such as ethyl acetate and the precipitated salt is collected by filteration. Then it is recrystallized and the salt thus obtained is dissolved in an acidic solution (e.g. 10% citric acid water solution), and finally extracted with a solvent such as ethyl acetate to give the optically active isomer of the compound (IV-2).

(6) Preparation of Sulfonyl Chloride

Sulfonyl chloride required as a raw material is prepared by the reaction of the corresponding benzene derivative or thiophene derivative with chlorosulfonic acid. The desired sulfonyl chloride can be obtained by reacting 1.0 mole of the benzene derivative or the thiophene derivative with 1-10 mole of chlorosulfonic acid in a solvent (e.g., 1,2-dichloroethane) with stirring at a temperature of −20-50° C. for 1-48 hours. Alternatively, it can be obtained by reacting the sulfonic acid derivative furthermore with thionyl chloride if a sulfonic acid derivative is obtained by the reaction of the benzene or thiophene derivative with chlorosulfonic acid.

According to this invention, not only the hydroxamic acid derivatives described above, but the following hydroxamic acid, which are already known, are preferably used as a compound inhibiting the solubilizing enzyme of HB-EGF.

1) [4-(N-hydroxyamino)-2 (R)-isobutyl-3-methylsuccinyl]-L-phenylglycine-N-methyl-amide (JP1995-101925A)

2) [4-(N-hydroxyamino)-2 (R)-isobutyl-3-methylsuccinyl]-L-3-(5,6,7,8-tetrahydro-1-naphtyl)alanine-N-methylamide (WO 97/09066)

3) [4-(N-hydroxyamino)-2 (R)-isobutylsuccinyl]-L-3-(1-naphtyl)alanine-N-methylamide (Bioorg. Med. Chem.,5, 765-778 (1997))

4) [4-(N-hydroxyamino)-2 (R)-isobutyl-3-(1,2,3,4-tetrahydroisoquinolylmethyl) succinyl]-L-phenylglycine —N-methylamide (Bioorg. Med. Chem.,5, 765-778 (1997))

5) 4-(N-hydroxyamino)-2 (R)-isopropyl-3-methylsuccinyl-L-phenylalanine-N-methyl amide (Drug design and Discovery, 16, 119-130 (1999))

6) 4-(N-hydroxyamino)-2 (R)-isobutyl-3-methylsuccinyl-L-phenylalanine-N-methyl amide (U.S. Pat. No. 4,743,587)

These hydroxamic acid derivatives 1)-6) are prepared by the method described each literature.

These compounds may be administered orally or parenterally to human.

The pharmaceutical preparations for oral administration include solid preparations such as tablets, granules, powders, fine granules, hard capsules, and solutions such as syrups, soft capsules. These preparations can be prepared by a conventional method. For example, tablets, granules, powders or fine granules are prepared by admixing the compound (I) or its pharmaceutically acceptable salt of the present invention with a conventional pharmaceutically acceptable carrier, such as lactose, starch, crystalline cellulose, magnesium stearate, hydroxy-propylcellulose, talc, etc., and the hard capsules can be prepared by filling the above fine granules or powders into suitable capsules. Besides, the syrups are prepared by dissolving or suspending the compound (I) or a pharmaceutically acceptable salt thereof in an aqueous solution containing sucrose, carboxycellulose, etc., and the soft capsules are prepared by dissolving or suspending the compound (I) or a pharmaceutically acceptable salt thereof in lipid excipients (e.g. vegetable oils, oily emulsion, glycol, etc.) and then filling the resultant into soft capsules.

The pharmaceutical preparations suitable for parenteral administration include injections and further percutaneous preparations (ointment, lotion or cream preparation), suppositories (e.g. suppository for rectal administration, suppository for vaginal administration), nasal preparation (e.g. spray preparation).

These preparations can be prepared by a conventional method. For example, the injections can be prepared by dissolving or emulsifying the compound (I) or a pharmaceutically acceptable salt thereof in a physiological saline or a lipid excipient (e.g. vegetable oil, oily emulsion, glycol, etc.) and then filling in an ampoule or vial with sealing under sterile condition. Ointment preparation is prepared by mixing the compound (I) or a pharmaceutically acceptable salt thereof with a base such as vaseline, paraffin or glycerin, if necessary hereto adding an emulsifier or a preservative, through a conventional method.

The dosage of the drug of this invention may vary depending on the preparations, ages, sexes, weights or conditions of the patients, but it is usually in the range of 0.1-600 mg/kg weight/day, or preferably 10-200 mg/kg weight/day of the compound (I), which is administered once a day or divided into 2 to 4 dosage units.

### EFFECTS OF THE INVENTION

The compounds of the present invention suppress the release of HB-EGF from cell membrane stimulated with 12-O-tetradecanoylphorbol-13-acetate (hereinafter, TPA)(see, Test example 1 below). They also suppress not only epidermal hyperplasia in mice induced by the application of TPA on their back (see, Test Example 2 below), but re-epithelialization of mouse skin wound model (see, Test Example 3 below). Remarkably severe side effects were not noted in Test Examples 2 and 3. Thus, the medicine according to the present invention is useful as an agent for suppressing the proliferation of keratinocytes.

### <TEST EXAMPLE 1>

1. Test Compounds

the compound a: [4-(N-hydroxyamino)-2 (R)-isobutyl-3-methylsuccinyl]-L-phenylglycine-N-methylamide (JP1995-101925A)

the compound b: [4-(N-hydroxyamino)-2 (R)-isobutyl-3-methylsuccinyl]-L-3-(5,6,7,8-tetrahydro-1-naphtyl)alanine-N-methylamide (WO 97/09066)

the compound c: [4-(N-hydroxyamino)-2 (R)-isobutyl-3-(1,2,3,4-tetrahydro-isoquinolylmethyl)succinyl]-L-phenylglycine —N-methylamide (Bioorg. Med. Chem.,5, 765-778 (1997))

the compound d: [4-(N-hydroxyamino)-2 (R)-isobutylsuccinyl]-L-3-(1-naphtyl)-alanine-N-methylamide (Bioorg. Med. Chem.,5, 765-778 (1997))

the compound e: 4-(N-hydroxyamino)-2 (R)-isopropyl-3-methylsuccinyl-L-phenylalanine-N-methylamide (Drug design and Discovery, 16, 119-130 (1999))

the compound f: 4-(N-hydroxyamino)-2 (R)-isobutyl-3-methylsuccinyl-L-phenylalanine-N-methylamide (U.S. Pat. No. 4,743,587)

the compound (1): (±)-N-Hydroxy-6-(4-methoxybenzenesulfonyl)-5,6,7,8-tetrahydropyrido[3,4-b]pyrazine-7-carboxamide (example 1)

the compound (2): (±)-N-Hydroxy-6-[4-(pyridine-4-yloxy)benzenesulfonyl]-5,6,7,8-tetrahydropyrido[3,4-b]pyrazine-7-carboxamide (example 2)

the compound (3): (±)-N-Hydroxy-6-(4-phenoxybenzenesulfonyl)-5,6,7,8-tetrahydropyrido[3,4-b]pyrazine-7-carboxamide (example 3)

the compound (4): (±)-N-Hydroxy-6-[4-(3-methoxypropoxy)benzenesulfonyl]-5,6,7,8-tetrahydropyrido[3,4-b]pyrazine-7-carboxamide (example 4)

the compound (5): (±)-N-Hydroxy-6-[4-(pyrazine-2-yloxy)benzenesulfonyl]-5,6,7,8-tetrahydropyrido[3,4-b]pyrazine-7-carboxamide (example 5)

the compound (6): (±)-N-Hydroxy-6-[4-(4-aminophenoxy)benzenesulfonyl]-5,6,7,8-tetrahydropyrido[3,4-b]pyrazine-7-carboxamide (example 6)

the compound (7): (±)-N-Hydroxy-6-[4-(2-ethoxyethoxy)benzenesulfonyl]-5,6,7,8-tetrahydropyrido[3,4-b]pyrazine-7-carboxamide (example 7)

the compound (8): (±)-N-Hydroxy-6-[4-(2-methoxyethoxy)benzenesulfonyl]-5,6,7,8-tetrahydropyrido[3,4-b]pyrazine-7-carboxamide (example 8)

the compound (9): (±)-N-Hydroxy-6-(4-pentyloxybenzenesulfonyl)-5,6,7,8-tetrahydropyrido[3,4-b]pyrazine-7-carboxamide (example 9)

the compound (10): (±)-N-Hydroxy-6-[4-(3-methylthiopropoxy)benzenesulfonyl]-5,6,7,8-tetrahydropyrido[3,4-b]pyrazine-7-carboxamide (example 10)

the compound (11): (±)-N-Hydroxy-6-[4-(methoxybutoxy)benzenesulfonyl]-5,6,7,8-tetrahydropyrido[3,4-b]pyrazine-7-carboxamide (example 11)

the compound (12): (±)-N-Hydroxy-6-[4-(ethoxypropoxy)benzenesulfonyl]-5,6,7,8-tetrahydropyrido[3,4-b]pyrazine-7-carboxamide (example 12)

the compound (13): (+)-N-Hydroxy-6-(4-methoxybenzenesulfonyl)-5,6,7,8-tetrahydropyrido[3,4-b]pyrazine-7-carboxamide (example 13)

the compound (14): (+)-N-Hydroxy-6-[4-(3-methoxypropoxy)benzenesulfonyl]-5,6,7,8-tetrahydropyrido[3,4-b]pyrazine-7-carboxamide (example 14)

the compound (15): (+)-N-Hydroxy-6-(4-ethoxybenzenesulfonyl)-5,6,7,8-tetrahydropyrido[3,4-b]pyrazine-7-carboxamide (example 15) the compound (16): (+)-N-Hydroxy-6-(4-propoxybenzenesulfonyl)-5,6,7,8-tetrahydropyrido[3,4-b]pyrazine-7-carboxamide (example 16)

the compound (17): (+)-N-Hydroxy-6-(4-trifluoromethoxybenzenesulfonyl)-5,6,7,8-tetrahydropyrido[3,4-b]pyrazine-7-carboxamide (example 17)

the compound (18): (+)-N-Hydroxy-6-[4-(2-fluoroethoxy)benzenesulfonyl]-5,6,7,8-tetrahydropyrido[3,4-b]pyrazine-7-carboxamide (example 18)

the compound (19): (+)-N-Hydroxy-6-[4-(2-methoxyethoxy)benzenesulfonyl]-5,6,7,8-tetrahydropyrido[3,4-b]pyrazine-7-carboxamide (example 19)

the compound (20): (+)-N-Hydroxy-6-(4-phenoxybenzenesulfonyl)-5,6,7,8-tetrahydropyrido[3,4-b]pyrazine-7-carboxamide (example 20)

the compound (21): (+)-N-Hydroxy-6-[4-(3-methylthiopropoxy)benzenesulfonyl]-5,6,7,8-tetrahydropyrido[3,4-b]pyrazine-7-carboxamide (example 21)

the compound (22): (+)-N-Hydroxy-6-(4-isobuthoxybenzenesulfonyl)-5,6,7,8-tetrahydropyrido[3,4-b]pyrazine-7-carboxamide (example 22)

the compound (23): (+)-N-Hydroxy-6-[4-(3-ethoxypropoxy)benzenesulfonyl]-5,6,7,8-tetrahydropyrido[3,4-b]pyrazine-7-carboxamide (example 23)

the compound (24): (+)-N-Hydroxy-6-[4-(3-bromopropoxy)benzenesulfonyl]-5,6,7,8-tetrahydropyrido[3,4-b]pyrazine-7-carboxamide (example 24)

the compound (25): (+)-N-Hydroxy-6-[4-(3-buthoxypropoxy)benzenesulfonyl]-5,6,7,8-tetrahydropyrido[3,4-b]pyrazine-7-carboxamide (example 25)

the compound (26): (+)-N-Hydroxy-6-[4-(3-isobuthoxypropoxy)benzenesulfonyl]-5,6,7,8-tetrahydropyrido[3,4-b]pyrazine-7-carboxamide (example 26)

the compound (27): (+)-N-Hydroxy-6-[4-(2-ethoxyethoxy)benzenesulfonyl]-5,6,7,8-tetrahydropyrido[3,4-b]pyrazine-7-carboxamide (example 27)

the compound (28): (+)-N-Hydroxy-6-(4-pivaloyloxybenzenesulfonyl)-5,6,7,8-tetrahydropyrido[3,4-b]pyrazine-7-carboxamide (example 31)

the compound (29): (+)-N-Hydroxy-6-[4-(2-cyanoethoxy)benzenesulfonyl]-5,6,7,8-tetrahydropyrido[3,4-b]pyrazine-7-carboxamide (example 32)

the compound (30): (+)-N-Hydroxy-6-(4-ethoxycarbonylmethoxy-benzenesulfonyl)-5,6,7,8-tetrahydropyrido[3,4-b]pyrazine-7-carboxamide (example 33)

the compound (31): (+)-N-Hydroxy-6-(4-methylbenzenesulfonyl)-5,6,7,8-tetrahydropyrido[3,4-b]pyrazine-7-carboxamide (example 34)

the compound (32): (+)-N-Hydroxy-6-[4-(3-ethoxycarbonylpropoxy)benzenesulfonyl]-5,6,7,8-tetrahydropyrido[3,4-b]pyrazine-7-carboxamide (example 35)

the compound (33): (+)-N-Hydroxy-6-[4-(2-acetoxyethoxy)benzenesulfonyl]-5,6,7,8-tetrahydropyrido[3,4-b]pyrazine-7-carboxamide (example 36)

the compound (34): (+)-N-Hydroxy-6-[4-(2-hydroxyethoxy)benzenesulfonyl]-5,6,7,8-tetrahydropyrido[3,4-b]pyrazine-7-carboxamide (example 37)

the compound (35): (+)-N-Hydroxy-6-(4-methylthiobenzenesulfonyl)-5,6,7,8-tetrahydropyrido[3,4-b]pyrazine-7-carboxamide (example 39)

the compound (36): (+)-N-Hydroxy-6-benzenesulfonyl-5,6,7,8-tetrahydropyrido[3,4-b]pyrazine-7-carboxamide (example 40)

the compound (37): (+)-N-Hydroxy-6-(4-fluorobenzenesulfonyl)-5,6,7,8-tetrahydropyrido[3,4-b]pyrazine-7-carboxamide (example 41)

the compound (38): (+)-N-Hydroxy-6-(4-hexylbenzenesulfonyl)-5,6,7,8-tetrahydropyrido[3,4-b]pyrazine-7-carboxamide (example 42)

the compound (39): (+)-N-Hydroxy-6-(4-aminobenzenesulfonyl)-5,6,7,8-tetrahydropyrido[3,4-b]pyrazine-7-carboxamide (example 43)

the compound (40): (+)-N-Hydroxy-6-(biphenyl-4-sulfonyl)-5,6,7,8-tetrahydropyrido[3,4-b]pyrazine-7-carboxamide (example 44)

the compound (41): (+)-N-Hydroxy-6-(4-hydroxybenzenesulfonyl)-5,6,7,8-tetrahydropyrido[3,4-b]pyrazine-7-carboxamide (example 45)

the compound (42): (+)-N-Hydroxy-6-(4-acetylbenzenesulfonyl)-5,6,7,8-tetrahydropyrido[3,4-b]pyrazine-7-carboxamide (example 46)

the compound (43): (+)-N-Hydroxy-6-(4-propylbenzenesulfonyl)-5,6,7,8-tetrahydropyrido[3,4-b]pyrazine-7-carboxamide (example 47)

the compound (44): (+)-N-Hydroxy-6-(4-vinylbenzenesulfonyl)-5,6,7,8-tetrahydropyrido[3,4-b]pyrazine-7-carboxamide (example 48)

the compound (45): (+)-N-Hydroxy-6-[4-(3-phenylpropoxy)benzenesulfonyl]-5,6,7,8-tetrahydropyrido[3,4-b]pyrazine-7-carboxamide (example 49)

the compound (46): (+)-N-Hydroxy-6-[(thiophen-2-yl)sulfonyl]-5,6,7,8-tetrahydropyrido[3,4-b]pyrazine-7-carboxamide (example 50)

the compound (47): (+)-N-Hydroxy-6-(2-trans-phenylethenesulfonyl)-5,6,7,8-tetrahydropyrido[3,4-b]pyrazine-7-carboxamide (example 51)

the compound (48): (+)-N-Hydroxy-6-[4-(3-methansulfonylpropoxy)benzenesulfonyl]-5,6,7,8-tetrahydropyrido[3,4-b]pyrazine-7-carboxamide (example 52)

the compound (49): 7,8-cis-N-Hydroxy-5,8-dimethyl-6-(4-methoxybenzenesulfonyl)-5,6,7,8-tetrahydropyrido[3,4-b]pyrazine-7-carboxamide (example 53)

2. Test Method

Human fibrosarcoma HT-1080 stable transfectant expressing fusion protein of HB-EGF and human placental alkaline phosphatase (AP) were used for following experiments.

The cultured transfectant was treated with Trypsin-EDTA solution, and suspended in Minimum Essential Medium (without phenol red; supplemented with 10% heat-inactivated fetal calf serum; MEM) at the concentration of 1×10⁵ cells/ml. Each 0.2 ml of the cell suspension was dispensed into individual wells of 96 well microplate, and incubated overnight in the CO₂ incubator (37° C.). After the incubation, the conditioned medium was removed, and the wells were washed once with 0.2 ml of phosphate buffered saline. The cells were treated with 0.1 ml of compound solution [compound solutions in dimethylsulfoxide (DMSO) were diluted 100 times with MEM] for 30 min at 37° C. Then the compound solutions were removed, and cells were treated with 0.2 ml of TPA solution (120 μM) of stock solution was diluted to 60 nM with MEM) containing same concentration of the compound for 60 min at 37° C. The cells treated same procedure without compound were used as control.

After these treatments, each 0.1 ml of conditioned medium was transferred to individual wells of new 96 well microplate and incubated at 65° C. for 10 min to inactivate endogenous alkaline phosphatase. One hundred microliter of substrate solution (1 mg/ml of p-nitrophenyl phosphate in 0.01% magnesium chloride/1 M diethanolamine) for AP was added to each well and incubated for 120 min at room temperature in the dark. The OD₄₀₅ of each well was measured with microplate reader. The IC₅₀ values of the compounds were calculated from the concentration-inhibition curves.

3. Test Results

The results are shown in Table 1

TABLE 1

| test compounds | IC₅₀(μM) |
|---|---|
| the compound a | 0.43 |
| the compound b | 0.12 |
| the compound c | 0.054 |
| the compound d | 1.7 |
| the compound e | 0.10 |
| the compound f | 0.10 |
| the compound(1) | 0.80 |
| the compound(2) | 0.99 |
| the compound(3) | 1.0 |
| the compound(4) | 0.24 |
| the compound(5) | 0.90 |
| the compound(6) | 1.1 |
| the compound(7) | 0.26 |
| the compound(8) | 0.84 |
| the compound(9) | 0.46 |
| the compound(10) | 0.34 |
| the compound(11) | 1.4 |
| the compound(12) | 0.16 |
| the compound(13) | 0.35 |
| the compound(14) | 0.053 |
| the compound(15) | 0.31 |
| the compound(16) | 1.0 |
| the compound(17) | 0.33 |
| the compound(18) | 0.16 |
| the compound(19) | 0.36 |
| the compound(20) | 0.41 |
| the compound(21) | 0.10 |
| the compound(22) | 3.6 |
| the compound(23) | 0.092 |
| the compound(24) | 0.60 |
| the compound(25) | 0.26 |
| the compound(26) | 0.56 |
| the compound(27) | 0.036 |
| the compound(28) | 2.8 |
| the compound(29) | 0.20 |
| the compound(30) | 1.0 |
| the compound(31) | 2.7 |
| the compound(32) | 1.1 |
| the compound(33) | 0.40 |
| the compound(34) | 1.5 |
| the compound(35) | 1.5 |
| the compound(36) | 0.51 |
| the compound(37) | 0.81 |
| the compound(38) | 1.7 |
| the compound(39) | 1.8 |
| the compound(40) | 1.5 |
| the compound(41) | 0.30 |
| the compound(42) | 2.2 |
| the compound(43) | 1.9 |
| the compound(44) | 1.1 |
| the compound(45) | 0.63 |
| the compound(46) | 0.96 |
| the compound(47) | 1.9 |
| the compound(48) | 1.8 |
| the compound(49) | 5.6 |

1. Test Compounds

the compound c: [4-(N-hydroxyamino)-2 (R)-isobutyl-3-(1,2,3,4-tetrahydro-isoquinolylmethyl)succinyl]-L-phenylglycine —N-methylamide (Bioorg. Med. Chem.,5, 765-778 (1997))

the compound d: [4-(N-hydroxyamino)-2 (R)-isobutylsuccinyl]-L-3-(1-naphtyl)-alanine-N-methylamide (Bioorg. Med. Chem.,5, 765-778 (1997))

the compound (13): (+)-N-Hydroxy-6-(4-methoxybenzenesulfonyl)-5,6,7,8-tetrahydropyrido[3,4-b]pyrazine-7-carboxamide (example 13)

the compound (14): (+)-N-Hydroxy-6-[4-(3-methoxypropoxy)benzenesulfonyl]-5,6,7,8-tetrahydropyrido[3,4-b]pyrazine-7-carboxamide (example 14)

the compound (15): (+)-N-Hydroxy-6-(4-ethoxybenzenesulfonyl)-5,6,7,8-tetrahydropyrido[3,4-b]pyrazine-7-carboxamide (example 15)

the compound (17): (+)-N-Hydroxy-6-(4-trifluoromethoxybenzenesulfonyl)-5,6,7,8-tetrahydropyrido[3,4-b]pyrazine-7-carboxamide (example 17)

the compound (19): (+)-N-Hydroxy-6-[4-(2-methoxyethoxy)benzenesulfonyl]-5,6,7,8-tetrahydropyrido[3,4-b]pyrazine-7-carboxamide (example 19)

the compound (20): (+)-N-Hydroxy-6-(4-phenoxybenzenesulfonyl)-5,6,7,8-tetrahydropyrido[3,4-b]pyrazine-7-carboxamide (example 20)

the compound (21): (+)-N-Hydroxy-6-[4-(3-methylthiopropoxy)benzenesulfonyl]-5,6,7,8-tetrahydropyrido[3,4-b]pyrazine-7-carboxamide (example 21)

the compound (27): (+)-N-Hydroxy-6-[4-(2-ethoxyethoxy)benzenesulfonyl]-5,6,7,8-tetrahydropyrido[3,4-b]pyrazine-7-carboxamide (example 27) the compound (36): (+)-N-Hydroxy-6-benzenesulfonyl-5,6,7,8-tetrahydropyrido[3,4-b]pyrazine-7-carboxamide (example 40)

the compound (37): (+)-N-Hydroxy-6-(4-fluorobenzenesulfonyl)-5,6,7,8-tetrahydropyrido[3,4-b]pyrazine-7-carboxamide (example 41)

the compound (40): (+)-N-Hydroxy-6-(biphenyl-4-sulfonyl)-5,6,7,8-tetrahydropyrido[3,4-b]pyrazine-7-carboxamide (example 44)

the compound (46): (+)-N-Hydroxy-6-[(thiophen-2-yl)sulfonyl]-5,6,7,8-tetrahydropyrido[3,4-b]pyrazine-7-carboxamide (example 50)

2. Test Method

After backs of male BALB/c mice were shaved, 20 micro 1 of TPA-acetone solution (50 μM) was applied to the dorsal skin surface in an area of ca. 1 cm². A volume of 20 micro 1 of test compound-acetone solution was applied to the same area a minute, 24 hours and 48 hours after TPA application (the treated group). In the non-treated group, acetone was applied instead of the test compound. In the control group, acetone was applied instead of TPA and the test compound. The mice were sacrificed, and the skin tissues treated with TPA or the test compound were excised 72 hours after TPA application. Then the tissues were fixed with 10% formalin and embedded in paraffin according to the conventional method. The vertical sections including the center of the TPA- or the test compound-treated area were prepared and they were stained by hematoxylin-eosin. The epidermal thickness was measured using an ocular microscope. The amount of the test compound required for achieving 50% inhibition against the mean epidermal thickness of the non-treated group (ED₅₀) was calculated, wherein the mean epidermal thickness of the non-treated group was 100% and that of the control group was 0%.

3. Test Result

The results are shown in Table 2

TABLE 2

| | Amount of test compound required for 50% |
|---|---|
| | suppression of epidermal hyperplasia |
| Test compound | (10⁻⁶ g/cm²) |
| the compound c | 190 |
| the compound d | 40 |
| the compound(13) | 350 |
| the compound(14) | 300 |
| the compound(15) | 370 |
| the compound(17) | 48 |
| the compound(19) | 200 |
| the compound(20) | 27 |
| the compound(21) | 230 |
| the compound(27) | 58 |
| the compound(36) | 130 |
| the compound(37) | 120 |
| the compound(40) | 20 |
| the compound(46) | 69 |

<Experiment 3>

1. Test Compounds

the compound (13): (+)-N-Hydroxy-6-(4-methoxybenzenesulfonyl)-5,6,7,8-tetrahydropyrido[3,4-b]pyrazine-7-carboxamide (example 13)

the compound (14): (+)-N-Hydroxy-6-[4-(3-methoxypropoxy)benzenesulfonyl]-5,6,7,8-tetrahydropyrido[3,4-b]pyrazine-7-carboxamide (example 14)

the compound (15): (+)-N-Hydroxy-6-(4-ethoxybenzenesulfonyl)-5,6,7,8-tetrahydropyrido[3,4-b]pyrazine-7-carboxamide (example 15)

the compound (16): (+)-N-Hydroxy-6-(4-propoxybenzenesulfonyl)-5,6,7,8-tetrahydropyrido[3,4-b]pyrazine-7-carboxamide (example 16)

the compound (19): (+)-N-Hydroxy-6-[4-(2-methoxyethoxy)benzenesulfonyl]-5,6,7,8-tetrahydropyrido[3,4-b]pyrazine-7-carboxamide (example 19)

the compound (20): (+)-N-Hydroxy-6-(4-phenoxybenzenesulfonyl)-5,6,7,8-tetrahydropyrido[3,4-b]pyrazine-7-carboxamide (example 20)

the compound (21): (+)-N-Hydroxy-6-[4-(3-methylthiopropoxy)benzenesulfonyl]-5,6,7,8-tetrahydropyrido[3,4-b]pyrazine-7-carboxamide (example 21)

2. Test Method

Wound healing model was prepared according to the method described by Tsuboi, et al. [J. Dermatol., 19, 673-675 (1992)]. Briefly, after backs of male BALB/c mice were shaved, two full-thickness round wounds were prepared in parallel to anterior-posterior axis on the back of each mouse using a punch biopsy instrument (6-mm diameter). After the operation, a test compound dissolved or suspended in 0.1M phosphate-buffered saline containing 1.5% sodium carboxymethylcellulose (vehicle) was applied to each wound in a volume of 50 μl/wound. The wounds were left open. From the following day, the compound was applied to each wound once a day for 7 days (the treated group). In the control group, vehicle solution only was applied in the same manner. One day after the last application, mice were sacrificed and skin tissues around the anterior wound were excised. The tissues were fixed with 10% formalin and embedded in paraffin according to the conventional method. The sections were made perpendicularly to the anterior-posterior axis. Keratinocytes in the specimen were stained with an anti-keratin antibody. Measurements of wound size and epidermal migration were performed by using an image-analyzing software. Re-epithelialization was calculated as follows;

$$Re \text{-} epithelialization ( \% ) = \frac{the length of re \text{-} epithelialized keratinocyte}{the length of wounded area} 100$$

The amount of the test compound required for achieving 50% inhibition against the mean re-epithelialization of the control group (ED₅₀) was calculated.

3. Test Result

TABLE 3

| Test compounds | ED₅₀ (10⁻⁶ g/0.3 cm²) |
|---|---|
| the compound(13) | 4.5 |
| the compound(14) | 9.8 |
| the compound(15) | 5.2 |
| the compound(16) | 11.6 |
| the compound(19) | 16.6 |
| the compound(20) | 9.7 |
| the compound(21) | 11.0 |

### EXAMPLES

The present invention is illustrated in more detail by the following Reference Examples and Examples.

### Example 1-58

Preparation of the 6-Sulfonyl-5,6,7,8-tetrapyrido[3,4-b]pyrazine derivatives (I)

Examples of the preparation are shown as follows.

method a: Preparation of (±)-N-Hydroxy-6-(4-methoxybenzenesulfonyl)-5,6,7,8-tetrahydropyrido[3,4-b]pyrazine-7-carboxamide

(±)-N-Benzyloxy-6-(4-methoxybenzenesulfonyl)-5,6,7,8-tetrahydropyrido[3,4-b]pyrazine-7-carboxamide (0.19 g) was dissolved in a mixture of methanol (5 ml) and tetrahydrofuran (5 ml), and the mixture was stirred in the presence of 10% Pd—C (0.10 g) under hydrogen atmosphere at room temperature for 2.5 hours. After removing Pd—C, the solvent was distilled off, and the residue was purified by HPLC (column; YMC-Pack ODS SH-343-5 S-5 120A, mobile phase; 0.1% aqueous trifluoroacetic acid solution/acetonitrile=4/1), and the purified fraction was lyophilized to give the title compound (28 mg) as colorless powders.

method b: Preparation of (±)-N-Hydroxy-6-[4-(3-methylthiopropoxy)benzene sulfonyl]-5,6,7,8-tetrahydropyrido[3,4-b]pyrazine-7-carboxamide

(±)-6-[4-(3-Methylthiopropoxy)benzenesulfonyl]-5,6,7,8-tetrahydropyrido[3,4-b]pyrazine-7-carboxylic acid (0.50 g) was dissolved in dichloromethane (5 ml), and thereto were added oxalyl chloride (0.18 g) and DMF (2 drops) under ice-cooling, and the mixture was stirred for one hour and then further stirred at room temperature overnight to give an acid chloride solution. To a mixture of a 50% aqueous hydroxylamine solution (1 ml) and 1,2-dimethoxyethane (3 ml) was added the above acid chloride solution, and the mixture was stirred at room temperature for 1.5 hour. The reaction mixture was extracted with chloroform, and the extract was washed with water and dried over magnesium sulfate. After removing magnesium sulfate, the solvent was distilled off, and the residue was purified by a silica gel column chromatography (mobile phase; chloroform/methanol=10/1) to give the title compound (0.15 g) as colorless powders.

TABLE 4

| a) | b) | Obtained compounds and their physical constants |
|---|---|---|
| 1 | A | (±)-N-Hydroxy-6-(4-methoxybenzenesulfonyl)-5,6,7,8-tetrahydro- |
| | | pyrido[3,4-b]pyrazine-7-carboxamide; |
| | | ¹H-NMR(250MHz, DMSO-d₆)δ; 2.95(dd, J=2.0 and 17.4Hz, 1H), 3.16(dd, J= |
| | | 7.0 and 17.4Hz, 1H), 3.82(s, 3H), 4.59(d, J=16.8Hz, 1H), 4.67(d, J=16.8Hz, |
| | | 1H), 4.78(dd, J=2.0 and 7.0Hz, 1H), 7.05(d, J=8.9Hz, 2H), 7.75(d, J=8.9Hz, |
| | | 2H), 8.30-8.50(m, 2H), 8.88(s, 1H), 10.91(s, 1H). |
| 2 | A | (±)-N-Hydroxy-6-[4-(pyridine-4-yloxy)benzenesulfonyl]-5,6,7,8- |
| | | tetrahydropyrido[3,4-b]pyrazine-7-carboxamide; |
| | | ¹H-NMR(250MHz, DMSO-d₆)δ; 2.98(dd, J=17.4 and 2.0Hz, 1H), 3.10-3.40(m, |
| | | 1H), 4.50-4.80(m, 2H), 4.79(dd, J=7.0 and 2.0Hz, 1H), 7.00(d, J=6.3Hz, 2H), |
| | | 7.29(d, J=8.8Hz, 2H), 7.91(d, J=8.8Hz, 2H), 8.43(s, 2H), 8.51(d, J=6.3Hz, 2H), |
| | | 8.90(s, 1H), 10.92(s, 1H). |
| 3 | A | (±)-N-Hydroxy-6-(4-phenoxybenzenesulfonyl)-5,6,7,8-tetrahydro- |
| | | pyrido[3,4-b]pyrazine-7-carboxamide; |
| | | ¹H-NMR(250MHz, DMSO-d₆)δ; 2.95(dd, J=17.5 and 1.8Hz, 1H), 3.18(dd, J=17.5 |
| | | and 7.1Hz, 1H), 4.50-4.70(m, 2H), 4.76(dd, J=7.1 and 1.8Hz, 1H), 7.03(d, |
| | | J=8.9Hz, 2H), 6.90-7.20(m, 2H), 7.10-7.30(m, 1H), 7.30-7.50(m, 2H), 7.79(d, |
| | | J=8.9Hz, 2H), 8.40(s, 2H), 8.88(s, 1H), 10.50-11.20(br, 1H). |
| 4 | A | (±)-N-Hydroxy-6-[4-(3-methoxypropoxy)benzenesulfonyl]-5,6,7,8- |
| | | tetrahydropyrido[3,4-b]pyrazine-7-carboxamide; |
| | | ¹H-NMR(250MHz, DMSO-d₆)δ; 1.94(tt, J=6.4 and 6.3Hz, 2H), 2.80-3.00(m, |
| | | 1H), 3.00-3.20(m, 1H), 3.23(s, 3H), 3.45(t, J=6.3Hz, 2H), 4.07(t, J=6.4Hz, 2H), |
| | | 4.50-4.70(m, 2H), 4.70-4.80(m, 1H), 7.04(d, J=8.9Hz, 2H), 7.73(d, J=8.9Hz, |
| | | 2H), 8.39(d, J=2.6Hz, 1H), 8.41(d, J=2.6Hz, 1H), 8.87(s, 1H), 10.91(s, 1H). |
| 5 | A | (±)-N-Hydroxy-6-[4-(pyrazine-2-yloxy)benzenesulfonyl]-5,6,7,8- |
| | | tetrahydropyrido[3,4-b]pyrazine-7-carboxamide; |
| | | ¹H-NMR(250MHz, DMSO-d₆)δ; 2.99(d, J=17.3Hz, 1H), 3.00-3.50(m, 1H), 4.63 |
| | | (d, J=16.7Hz, 1H), 4.71(d, J=16.7Hz, 1H), 4.81(d, J=5.1Hz, 1H), 7.36(d, J=8.7Hz, |
| | | 2H), 7.89(d, J=8.7Hz, 2H), 8.20-8.30(m, 1H), 8.30-8.50(m, 3H), 8.50-8.60 |
| | | (m, 1H), 8.90(s, 1H), 10.92(s, 1H). |
| 6 | A | (±)-N-Hydroxy-6-[4-(4-aminophenoxy)benzenesulfonyl]-5,6,7,8- |
| | | tetrahydropyrido[3,4-b]pyrazine-7-carboxamide; |
| | | ¹H-NMR(250MHz, DMSO-d₆)δ; 2.96(dd, J=2.0 and 17.4Hz, 1H), 3.10-3.30(m, |
| | | 1H), 4.50-4.70(m, 2H), 4.70-4.80(m, 1H), 5.00-5.10(m, 2H), 6.61(d, J=8.8Hz, |
| | | 2H), 6.79(d, J=8.8Hz, 2H), 6.93(d, J=8.9Hz, 2H), 7.75(d, J=8.9Hz, 2H), 8.42(s, |
| | | 2H), 8.90(s, 1H), 10.90(s, 1H). |
| 7 | A | (±)-N-Hydroxy-6-[4-(2-ethoxyethoxy)benzenesulfonyl]-5,6,7,8-tetra-hydro- |
| | | pyrido[3,4-b]pyrazine-7-carboxamide; |
| | | ¹H-NMR(250MHz, DMSO-d₆)δ; 1.11(t, J=7.0Hz, 3H), 2.96(d, J=17.4Hz, 1H), |
| | | 3.14(dd, J=6.7 and 17.4Hz, 1H), 3.48(q, J=7.0Hz, 2H), 3.60-3.70(m, 2H), 4.10-4.20 |
| | | (m, 2H), 4.60(d, J=17.4Hz, 1H), 4.68(d, J=17.4Hz, 1H), 4.78(d, J=6.7Hz, |
| | | 1H), 7.05(d, J=8.8Hz, 2H), 7.74(d, J=8.8Hz, 2H), 8.89(brs, 1H), 10.92(br s, |
| | | 1H). |
| 8 | A | (±)-N-Hydroxy-6-[4-(2-methoxyethoxy)benzenesulfonyl]-5,6,7,8- |
| | | tetrahydropyrido[3,4-b]pyrazine-7-carboxamide; |
| | | ¹H-NMR(250MHz, DMSO-d₆)δ; 2.95(d, J=17.3Hz, 1H), 3.00-3.20(m, 1H), 3.28 |
| | | (s, 3H), 3.60-3.70(m, 2H), 4.10-4.20(m, 2H), 4.59(d, J=17.0Hz, 1H), 4.67(d, |
| | | J=17.0Hz, 1H), 4.76(d, J=6.9Hz, 1H), 7.05(d, J=8.8Hz, 2H), 7.73(d, J=8.8Hz, |
| | | 2H), 8.30-8.50(m, 2H), 8.87(s, 1H), 10.91(s, 1H). |
| 9 | A | (±)-N-Hydroxy-6-(4-pentyloxybenzenesulfonyl)-5,6,7,8-tetrahydro- |
| | | pyrido[3,4-b]pyrazine-7-carboxamide; |
| | | ¹H-NMR(250MHz, DMSO-d₆)δ; 0.89(t, J=7.1Hz, 3H), 1.20-1.50(m, 4H), 1.60-1.80 |
| | | (m, 2H), 2.96(d, J=17.3Hz, 1H), 3.16(dd, J=6.6 and 17.3Hz, 1H), 4.02(t, |
| | | J=6.5Hz, 2H), 4.59(d, J=16.7Hz, 1H), 4.67(d, J=16.7Hz, 1H), 4.70-4.80(m, 1H), |
| | | 7.03(d, J=8.9Hz, 2H), 7.73(d, J=8.9Hz, 2H), 8.30-8.50(m, 2H), 8.88(brs, 1H), |
| | | 10.91(br s, 1H). |
| 10 | B | (±)-N-Hydroxy-6-[4-(3-methylthiopropoxy)benzenesulfonyl]-5,6,7,8-tetra- |
| | | hydropyrido[3,4-b]pyrazine-7-carboxamide; |
| | | ¹H-NMR(250MHz, CDCl₃)δ; 1.80-2.00(m, 2H), 2.00(s, 3H), 2.54(t, J=7.2Hz, |
| | | 2H), 2.90(d, J=17.3Hz, 1H), 3.09(dd, J=7.0 and 17.3Hz, 1H), 4.05(t, J=6.2Hz, |
| | | 2H), 4.53(d, J=16.8Hz, 1H), 4.62(d, J=16.8Hz, 1H), 4.72(d, J=7.0Hz, 1H), 6.98 |
| | | (d, J=8.8Hz, 2H), 7.68(d, J=8.8Hz, 2H), 8.30-8.40(m, 2H), 8.82(s, 1H), 10.86 |
| | | (s, 1H). |
| 11 | A | (±)-N-Hydroxy-6-[4-(4-methoxybutoxy)benzenesulfonyl]-5,6,7,8-tetrahydro- |
| | | pyrido[3,4-b]pyrazine-7-carboxamide; |
| | | ¹H-NMR(250MHz, DMSO-d₆)δ; 1.50-1.90(m, 4H), 2.96(d, J=17.5Hz, 1H), 3.16 |
| | | (dd, J=6.7 and 17.5Hz, 1H), 3.23(s, 3H), 3.36(t, J=6.2Hz, 2H), 4.05(t, J=6.3Hz, |
| | | 2H), 4.59(d, J=17.0Hz, 1H), 4.68(d, J=17.0Hz, 1H), 4.70-4.90(m, 1H), 7.04 |
| | | (d, J=8.9Hz, 2H), 7.74(d, J=8.9Hz, 2H), 8.30-8.50(m, 2H), 8.88(brs, 1H), 10.92 |
| | | (br s, 1H). |
| 12 | A | (±)-N-Hydroxy-6-[4-(3-ethoxypropoxy)benzenesulfonyl]-5,6,7,8-tetrahydro- |
| | | pyrido[3,4-b]pyrazine-7-carboxamide; |
| | | ¹H-NMR(250MHz, DMSO-d₆)δ; 1.09(t, J=7.0Hz, 3H), 1.93(q, J=6.3Hz, 2H), |
| | | 2.90-3.00(m, 1H), 3.10-3.30(m, 1H), 3.40(q, J=7.0Hz, 2H), 3.48(t, J=6.3Hz, |
| | | 2H), 4.08(t, J=6.3Hz, 2H), 4.50-4.70(m, 2H), 4.70-4.80(m, 1H), 7.04(d, J=8.9Hz, |
| | | 2H), 7.73(d, J=8.9Hz, 2H), 8.38(d, J=2.6Hz, 1H), 8.41(d, J=2.6Hz, 1H), 8.87 |
| | | (s, 1H), 10.90(s, 1H). |
| 13 | A | (+)-N-Hydroxy-6-(4-methoxybenzenesulfonyl)-5,6,7,8-tetrahydropyrido- |
| | | [3,4-b]pyrazine-7-carboxamide; |
| | | ¹H-NMR(250MHz, DMSO-d₆)δ; 2.95(dd, J=17.4 and 2.0Hz, 1H), 3.16(dd, J= |
| | | 17.4 and 7.0Hz, 1H), 3.82(s, 3H), 4.59(d, J=16.8Hz, 1H), 4.67(d, J=16.8Hz, |
| | | 1H), 4.78(dd, J=7.0 and 2.0Hz, 1H), 7.05(d, J=8.9Hz, 2H), 7.75(d, J=8.9Hz, |
| | | 2H), 8.30-8.50(m, 2H), 8.88(s, 1H), 10.91(s, 1H). |
| | | MALDI-TOF MS(M.W.364.38): 365[M+H]⁺, 387[M+Na]⁺, 403[M+K]⁺. |
| | | [α]_{D}; +30°(C=1.0,MeOH). |
| 14 | A | (+)-N-Hydroxy-6-[4-(3-methoxypropoxy)benzenesulfonyl]-5,6,7,8-tetrahydro |
| | | pyrido[3,4-b]pyrazine-7-carboxamide; |
| | | ¹H-NMR(250MHz, DMSO-d₆)δ; 1.94(tt, J=6.4 and 6.3Hz,2H), 2.80-3.00(m, |
| | | 1H), 3.00-3.20(m, 1H), 3.23(s, 3H), 3.45(t, J=6.3Hz, 2H), 4.07(t, J=6.4Hz, 2H), |
| | | 4.50-4.70(m, 2H), 4.70-4.80(m, 1H), 7.04(d, J=8.9Hz, 2H), 7.73(d, J=8.9Hz, |
| | | 2H), 8.39(d, J=2.6Hz, 1H), 8.41(d, J=2.6Hz, 1H), 8.87(s, 1H), 10.91(s, 1H). |
| | | MALDI-TOF MS(M.W.422.46): 423[M+H]⁺, 445[M+Na]⁺, 461[M+K]⁺ |
| 15 | A | (+)-N-Hydroxy-6-(4-ethoxybenzenesulfonyl)-5,6,7,8-tetrahydro- |
| | | pyrido[3,4-b]pyrazine-7-carboxamide; |
| | | ¹H-NMR(250MHz, DMSO-d₆)δ; 1.32(t, J=7.0Hz, 3H), 2.95(dd, J=17.4 and 1.8Hz, |
| | | 1H), 3.14(dd, J=17.4 and 6.9Hz, 1H), 4.08(q, J=7.0Hz, 2H), 4.59(d, J=16.8Hz, |
| | | 1H), 4.67(d, J=16.8Hz, 1H), 4.77(dd, J=6.9 and 1.8Hz, 1H), 7.02(d, J=9.0Hz, |
| | | 2H), 7.73(d, J=9.0Hz, 2H), 8.38(d, J=2.6Hz, 1H), 8.41(d, J=2.6Hz, 1H), 8.88 |
| | | (s, 1H), 10.91(s, 1H). |
| | | MALDI-TOF MS(M.W.378.41): 379[M+H]⁺, 401[M+Na]⁺, 417[M+K]⁺ |
| 16 | A | (+)-N-Hydroxy-6-(4-propoxybenzenesulfonyl)-5,6,7,8-tetrahydro- |
| | | pyrido[3,4-b]pyrazine-7-carboxamide; |
| | | ¹H-NMR(250MHz, DMSO-d₆)δ; 0.95(t, J=7.4Hz, 3H), 1.60-1.80(m, H), 2.95 |
| | | (dd, J=17.2 and 1.8Hz, 1H), 3.15(dd, J=17.2 and 6.9Hz, 1H), 3.98(t, J=6.5Hz, |
| | | 2H), 4.58(d, J=16.9Hz, 1H), 4.67(d, J=16.9Hz, 1H), 4.77(dd, J=6.9 and 1.8Hz, |
| | | 1H), 7.03(d, J=8.9Hz, 2H), 7.73(d, J=8.9Hz, 2H), 8.38(d, J=2.5Hz, 1H), 8.41 |
| | | (d, J=2.5Hz, 1H), 8.88(s, 1H), 10.92(s, 1H). |
| | | MALDI-TOF MS(M.W.392.43): 393[M+H]⁺, 415[M+Na]⁺, 431[M+K]⁺ |
| 17 | A | (+)-N-Hydroxy-6-(4-trifluoromethoxybenzenesulfonyl)-5,6,7,8- |
| | | tetrahydropyrido[3,4-b]pyrazine-7-carboxamide; |
| | | ¹H-NMR(250MHz, DMSO-d₆)δ; 2.99(dd, J=2.0 and 17.3Hz, 1H), 3.22(dd, J= |
| | | 7.0 and 17.3Hz, 1H), 4.62(d, J=16.6Hz, 1H), 4.72(d, J=16.6Hz, 1H), 4.80(dd, |
| | | J=2.0 and 7.0Hz, 1H), 7.52(d, J=8.9Hz, 2H), 7.96(d, J=8.9Hz, 2H), 8.38(d, J=2.6Hz, |
| | | 1H), 8.40(d, J=2.6Hz, 1H), 8.89(s, 1H), 10.92(s, 1H). |
| | | MALDI-TOF MS(M.W.418.35): 419[M+H]⁺, 441[M+Na]⁺, |
| | | 457[M+K]⁺. |
| 18 | A | (+)-N-Hydroxy-6-[4-(2-fluoroethoxy)benzenesulfonyl]-5,6,7,8-tetra- |
| | | hydropyrido[3,4-b]pyrazine-7-carboxamide; |
| | | ¹H-NMR(250MHz, DMSO-d₆)δ; 2.94(d, J=17.3Hz, 1H), 3.15(dd, J=7.0 and 17.3Hz, |
| | | 1H), 4.20-4.40(m, 2H), 4.50-4.90(m, 5H), 7.07(d, J=8.9H, 2H), 7.75(d, |
| | | J=8.9Hz, 2H), 8.38(d, J=2.5Hz, 1H), 8.40(d, J=2.5Hz, 1H), 8.87(s, 1H), 10.91 |
| | | (s, 1H). |
| 19 | A | (+)-N-Hydroxy-6-[4-(2-methoxyethoxy)benzenesulfonyl]-5,6,7,8- |
| | | tetrahydropyrido[3,4-b]pyrazine-7-carboxamide; |
| | | ¹H-NMR(250MHz, DMSO-d₆)δ; 2.95(d, J=17.3Hz, 1H), 3.00-3.20(m, 1H), 3.28 |
| | | (s, 3H), 3.60-3.70(m, 2H), 4.10-4.20(m, 2H), 4.59(d, J=17.0Hz, 1H), 4.67(d, |
| | | J=17.0Hz, 1H), 4.76(d, J=6.9Hz, 1H), 7.05(d, J=8.8Hz, 2H), 7.73(d, J=8.8Hz, |
| | | 2H), 8.30-8.50(m, 2H), 8.87(s, 1H), 10.91(s, 1H). |
| | | MALDI-TOF MS(M.W.408.43): 409[M+H]⁺, 431[M+Na]⁺, 447[M+K]⁺ |
| 20 | A | (+)-N-Hydroxy-6-(4-phenoxybenzenesulfonyl)-5,6,7,8-tetrahydro- |
| | | pyrido[3,4-b]pyrazine-7-carboxamide; |
| | | ¹ H-NMR(250MHz, DMSO-d₆)δ; 2.96(d, J=17.2Hz, 1H), 3.20(dd, J=7.3 and 17.2Hz, |
| | | 1H), 4.50-4.70(m, 2H), 4.70-4.80(m, 1H), 7.00-7.20(m, 4H), 7.20-7.30 |
| | | (m, 1H), 7.40-7.50(m, 2H), 7.70-7.90(m, 2H), 8.42(s, 2H), 8.92(s, 1H), 10.94 |
| | | (s, 1H). |
| | | MALDI-TOF MS(M.W.426.45): 427[M+H]⁺, 449[M+Na]⁺, 465[M+K]⁺ |
| | | [α]_{D}; +10°(C=0.25,MeOH) |
| 21 | B | (+)-N-Hydroxy- 6-[4-(3-methylthiopropoxy)benzenesulfonyl]-5,6,7,8- |
| | | tetrahydropyrido[3,4-b]pyrazine-7-carboxamide; |
| | | ¹H-NMR(250MHz, DMSO-d₆)δ; 1.90-2.10(m, 2H), 2.05(s, 3H), 2.60(t, J=7.2Hz, |
| | | 2H), 2.94(d, J=17.5Hz, 1H), 3.15(dd, J=7.1 and 17.5Hz, 1H), 4.10(t, J=6.2Hz, |
| | | 2H), 4.57(d, J=16.8Hz, 1H), 4.67(d, J=16.8Hz, 1H), 7.04(d, J=8.8Hz, 2H), |
| | | 7.73(d, J=8.8Hz, 2H), 8.40-8.50(m, 2H), 8.91(s, 1H), 10.94(s, 1H). |
| | | MALDI-TOF MS(M.W.438.53): 439[M+H]⁺, 461[M+Na]⁺, 477[M+K]⁺. |
| | | α]_{D}; +20°(C=0.5,MeOH) |
| 22 | A | (+)-N-Hydroxy-6-(4-isobutoxybenzenesulfonyl)-5,6,7,8-tetrahydro- |
| | | pyrido[3,4-b]pyrazine-7-carboxamide; |
| | | ¹H-NMR(250MHz, DMSO-d₆)δ; 0.95(d, J=6.7Hz, 6H), 1.90-2.10(m, 1H), 2.9 |
| | | 4(d, J=17.2Hz, 1H), 3.15(dd, J=6.8 and 17.2Hz, 1H), 3.79(d, J=6.5Hz, 2H), 4.5 |
| | | 7(d, J=16.7Hz, 1H), 4.66(d, J=16.7Hz, 1H), 4.77(d, J=6.8Hz, 1H), 7.03(d, J=8.8Hz, |
| | | 2H), 7.72(d, J=8.8Hz, 2H), 8.30-8.50(m, 2H), 8.91(brs, 1H), 10.95(brs, |
| | | 1H). |
| 23 | B | (+)-N-Hydroxy-6-[4-(3-ethoxypropoxy)benzenesulfonyl]-5,6,7,8- |
| | | tetrahydropyrido[3,4-b]pyrazine-7-carboxamide; |
| | | ¹H-NMR(250MHz, DMSO-d₆)δ; 1.08(t, J=7.0Hz, 3H), 1.80-2.00(m, 2H), 2.93 |
| | | (d, J=17.4Hz, 1H), 3.15(dd, J=17.4 and 6.6Hz, 1H), 3.39(q, J=7.0Hz, 2H), 3.47 |
| | | (t, J=6.3Hz, 2H), 4.07(t, J=6.3Hz, 2H), 4.57(d, J=17.0Hz, 1H), 4.69(d, J=17.0Hz,) |
| | | 1H), 4.70-4.80(m, 1H), 7.03(d, J=8.9Hz, 2H), 7.72(d, J=8.9Hz, 2H), 8.38(d, |
| | | J=2.6Hz, 1H), 8.41(d, J=2.6Hz, 1H), 8.90(s, 1H), 10.93(s, 1H). |
| | | MALDI-TOF MS(M.W.436.49): 437[M+H]⁺, 459[M+Na]⁺, 475[M+K]⁺. |
| 24 | B | (+)-N-Hydroxy-6-[4-(3-bromopropoxy)benzenesulfonyl]-5,6,7,8- |
| | | tetrahydropyrido[3,4-b]pyrazine-7-carboxamide; |
| | | ¹H-NMR(250MHz, DMSO-d₆)δ; 2.20-2.40(m, 2H), 2.95(dd, J=1.7 and 17.5Hz, |
| | | 1H), 3.17(dd, J=6.8 and 17.5Hz, 1H), 3.66(t, J=6.6Hz, 2H), 4.14(t, J=6.0Hz, |
| | | 2H), 4.59(d, J=16.7Hz, 1H), 4.68(d, J=16.7Hz, 1H), 4.78(dd, J=1.7 and 6.8Hz, |
| | | 1H), 7.00-7.20(m, 2H), 7.70-7.80(m, 2H), 8.40(d, J=2.6Hz, 1H), 8.42(d, J=2.6Hz, |
| | | 1H), 8.91(s, 1H), 10.95(s, 1H). |
| 25 | A | (+)-N-Hydroxy-6-[4-(3-butoxypropoxy)benzenesulfonyl]-5,6,7,8- |
| | | tetrahydrapyrido[3,4-b]pyrazine-7-carboxamide; |
| | | ¹H-NMR(250MHz, DMSO-d₆)δ; 0.83(t, J=7.3Hz, 3H), 1.20-1.60(m, 4H), 1.80- |
| | | 2.00(m, 2H), 2.90-3.00(m, 1H), 3.14(dd, J=7.0 and 17.4Hz, 1H), 3.30-3.40 |
| | | (m, 2H), 3.47(t, J=6.3Hz, 2H), 4.07(t, J=6.3Hz, 2H), 4.57(d, J=17.2Hz, 1H), 4.66 |
| | | (d, 17.2Hz, 1H), 4.70-4.80(m, 1H), 7.03(d, J=8.9Hz, 2H), 7.73(d, J=8.9Hz, |
| | | 2H), 8.30-8.50(m, 2H), 8.90(s, 1H), 10.94(s, 1H). |
| 26 | A | (+)-N-Hydroxy-6-[4-(3-isobutoxypropoxy)benzenesulfonyl]-5,6,7,8- |
| | | tetrahydropyrido[3,4-b]pyrazine-7-carboxamide; |
| | | ¹H-NMR(250MHz, DMSO-d₆)δ; 0.82(d, J=6.7Hz, 6H), 1.60-2.00(m, 3H), 2.80- |
| | | 3.00(m, 1H), 3.00-3.30(m, 3H), 3.48(t, J=6.2Hz, 2H), 4.08(t, J=6.3Hz, 2H), |
| | | 4.58(d, J=17.1Hz, 1H), 4.66(d, 17.1Hz, 1H), 4.76(dd, J=2.1 and 7.1Hz, 1H), 7.03 |
| | | (d, J=8.9Hz, 2H), 7.73(d, J=8.9Hz, 2H), 8.30-8.50(m, 2H), 8.90(s, 1H), 10.94 |
| | | (s, 1H). |
| 27 | B | (+)-N-Hydroxy-6-[4-(2-ethoxyethoxy)benzenesulfonyl]-5,6,7,8-tetra- |
| | | hydropyrido[3,4-b]pyrazine-7-carboxamide; |
| | | ¹H-NMR(250MHz, DMSO-d₆)δ; 1.09(t, J=7.0Hz, 3H), 2.93(dd, J=1.8 and 17.4Hz, |
| | | 1H), 3.14(dd, J=7.0 and 17.4Hz, 1H), 3.46(q, J=7.0Hz, 2H), 3.60-3.80(m, |
| | | 2H), 4.00-4.20(m, 2H), 4.57(d, J=16.8Hz, 1H), 4.66(d, 16.8Hz, 1H), 4.75(dd, J= |
| | | 7.0 and 1.8Hz, 1H), 7.04(d, J=9.0Hz, 2H), 7.72(d, J=9.0Hz, 2H), 8.30-8.50 |
| | | (m, 2H), 8.86(brs, 1H), 10.91(brs, 1H). |
| | | MALDI-TOF MS(M.W.422.46): 423[M+H]⁺, 445[M+Na]⁺, 461[M+K]⁺. |
| 28 | B | (+)-N-Hydroxy-6-[4-(pyrazine-2-yloxy)benzenesulfonyl]-5,6,7,8- |
| | | tetrahydropyrido[3,4-b]pyrazine-7-carboxamide; |
| | | ¹H-NMR(250MHz, DMSO-d₆)δ; 2.90-3.10(m, 1H), 3.23(dd, J=7.0 and 17.5Hz, |
| | | 1H), 4.64(d, J=17.0Hz, 1H), 4.74(d, J=17.0Hz, 1H), 4.80-4.90(m, 1H), 7.39 |
| | | (d, J=8.8Hz, 2H), 7.91(d, J=8.8Hz, 2H), 8.27(dd, J=1.3 and 2.7Hz, 1H), 8.40-8.50 |
| | | (m, 3H), 8.61(d, J=1.3Hz, 1H), 8.95(brs, 1H), 10.97(s, 1H). |
| | | MALDI-TOF MS(M.W.428.43): 429[M+H]⁺, 451[M+Na]⁺, 467[M+K]⁺ |
| 29 | B | (+)-N-Hydroxy-6-(4-pentyloxybenzenesulfonyl)-5,6,7,8-tetrahydro- |
| | | pyrido[3,4-b]pyrazine-7-carboxamide; |
| | | ¹H-NMR(250MHz, DMSO-d₆)δ; 0.88(t, J=7.1Hz, 3H), 1.20-1.50(m, 4H), 1.60-1.80 |
| | | (m, 2H), 2.80-3.00(m, 1H), 3.05-3.30(m, 1H), 4.01(t, J=6.4Hz, 2H), 4.58 |
| | | (d, J=16.5Hz, 1H), 4.66(d, J=16.5Hz, 1H), 4.70-4.80(m, 1H), 7.03(d, J=8.9Hz, |
| | | 2H), 7.72(d, J=8.9Hz, 2H), 8.30-8.50(m, 2H), 8.90(brs, 1H), 10.93(brs, 1H). |
| | | MALDI-TOF MS(M.W.420.49): 421[M+H]⁺, 443[M+Na]⁺, 459[N+K]⁺ |
| 30 | A | (+)-N-Hydroxy-6-[4-(4-aminophenoxy)benzenesulfonyl]-5,6,7,8- |
| | | tetrahydropyrido[3,4-b]pyrazine-7-carboxamide; |
| | | ¹H-NMR(250MHz, DMSO-d₆)δ; 2.95(dd, J=1.7 and 17.3Hz, 1H), 3.10-3.30 |
| | | (m, 1H), 4.50-4.70(m, 2H), 4.76(dd, J=1.7 and 6.8Hz, 1H), 5.10(brs, 2H), 6.60 |
| | | (d, J=8.8Hz, 2H), 6.79(d, J=8.8Hz, 2H), 6.91(d, J=8.9Hz, 2H), 7.74(d, J=8.9Hz, |
| | | 2H), 8.30-8.50(m, 2H), 8.92(brs, 1H), 10.93(brs, 1H). |
| 31 | A | (+)-N-Hydroxy-6-(4-pivaloyloxybenzenesulfonyl)-5,6,7,8-tetra- |
| | | hydropyrido[3,4-b]pyrazine-7-carboxamide; |
| | | ¹H-NMR(250MHz, DMSO-d₆)δ; 1.31(s, 9H), 2.99(d, J=15.7Hz, 1H), 3.10-3.30 |
| | | (m, 1H), 4.50-4.90(m, 3H), 7.33(d, J=8.5Hz, 2H), 7.90(d, J=8.5Hz, 2H), 8.30-8.50 |
| | | (m, 2H), 8.93(brs, 1H), 10.97(brs, 1H). |
| 32 | A | (+)-N-Hydroxy-6-[4-(2-cyanoethoxy)benzenesulfonyl]-5,6,7,8-tetra- |
| | | hydropyrido[3,4-b]pyrazine-7-carboxamide; |
| | | ¹H-NMR(250MHz, DMSO-d₆)δ; 2.90-3.10(m, 3H), 3.17(dd, J=7.3 and 17.3Hz, |
| | | 1H), 4.26(t, J=6.0Hz, 2H), 4.60(d, J=16.7Hz, 1H), 4.68(d, J=16.7Hz, 1H), 4.70-4.90 |
| | | (m, 1H), 7.10(d, J=8.9Hz, 2H), 7.77(d, J=8.9Hz, 2H), 8.30-8.50(m, |
| | | 2H), 8.88(brs, 1H), 10.88(brs, 1H). |
| | | MALDI-TOF MS(M.W.403.42): 404[M+H]⁺, 426[M+Na]⁺, 442[M+K]⁺ |
| 33 | A | (+)-N-Hydroxy-6-(4-ethoxycarbonylmethoxybenzenesulfonyl)-5,6,7,8-tetrahydropyrido |
| | | [3,4-b]pyrazine-7-carboxamide; |
| | | ¹H-NMR(250MHz, DMSO-d₆)δ; 1.20(t, J=7.1Hz, 3H), 2.90-3.10(m, 1H), 3.15 |
| | | (dd, J=6.7 and 17.1Hz, 1H), 4.16(q, J=7.1Hz, 2H), 4.59(d, J=16.6Hz, 1H), 4.68 |
| | | (d, J=16.6Hz, 1H), 4.70-4.85(m, 1H), 4.88(s, 2H), 7.06(d, J=9.0Hz, 2H), 7.76 |
| | | (d, J=9.0Hz, 2H), 8.30-8.50(m, 2H), 8.92(brs, 1H), 10.95(brs, 1H). |
| 34 | A | (+)-N-Hydroxy-6-(4-methylbenzenesulfonyl)-5,6,7,8-tetrahydro- |
| | | pyrido[3,4-b]pyrazine-7-carboxamide; |
| | | ¹H-NMR(250MHz, DMSO-d₆)δ; 2.34(s, 3H), 2.93(dd, J=1.8 and 17.3Hz, 1H), |
| | | 3.14(dd, J=6.8 and 17.3Hz, 1H), 4.58(d, J=16.8Hz, 1H), 4.67(d, J=16.8Hz, |
| | | 1H), 4.78(dd, J=1.8 and 6.8Hz, 1H), 7.34(d, J=8.2Hz, 2H), 7.69(d, J=8.2Hz, |
| | | 2H), 8.30-8.50(m, 2H), 8.91(brs, 1H), 10.94(brs, 1H). |
| 35 | A | (+)-N-Hydroxy-6-[4-(3-ethoxycarbonylpropoxy)benzenesulfonyl]-5,6,7,8-tetrahydropyrido |
| | | [3,4-b]pyrazine-7-carboxamide; |
| | | ¹H-NMR(250MHz, DMSO-d₆)δ; 1.17(t, J=7.1Hz, 3H), 1.90-2.10(m, 2H), 2.45 |
| | | (t, J=7.2Hz, 2H), 2.90-3.00(m, 1H), 3.16(dd, J=7.0 and 17.3Hz, 1H), 4.05(t, J= |
| | | 6.3Hz, 2H), 4.06(q, J=7.1Hz, 2H), 4.58(d, J=16.6Hz, 1H), 4.67(d, 16.6Hz, 1H), |
| | | 4.70-4.80(m, 1H), 7.04(d, J=8.9Hz, 2H), 7.74(d, J=8.9Hz, 2H), 8.30-8.50(m, |
| | | 2H), 8.92(brs, 1H), 10.95(brs, 1H). |
| 36 | A | (+)-N-Hydroxy-6-[4-(2-acetoxyethoxy)benzenesulfonyl]-5,6,7,8- |
| | | tetrahydropyrido[3,4-b]pyrazine-7-carboxamide; |
| | | ¹H-NMR(250MHz, DMSO-d₆)δ; 2.03(s, 3H), 2.90-3.10(m, 1H), 3.17(dd, J=17.3 |
| | | and 7.1Hz, 1H), 4.20-4.40(m, 4H), 4.59(d, J=16.7Hz, 1H), 4.68(d, J=16.7Hz, |
| | | 1H), 4.70-4.90(m, 1H), 7.08(d, J=8.9Hz, 2H), 7.76(d, J=8.9Hz, 2H), 8.30-8.50 |
| | | (m, 2H), 8.91(brs, 1H), 10.95(brs, 1H). |
| 37 | A | (+)-N-Hydroxy-6-[4-(2-hydroxyethoxy)benzenesulfonyl]-5,6,7,8- |
| | | tetrahydropyrido[3,4-b]pyrazine-7-carboxamide; |
| | | ¹H-NMR(250MHz, DMSO-d₆)δ; 2.90-3.00(m, 1H), 3.16(dd, J=17.4 and 6.9Hz, |
| | | 1H), 3.60-3.80(m, 2H), 4.05(t, J=4.8Hz, 2H), 4.59(d, J=16.8Hz, 1H), 4.68(d, |
| | | J=16.8Hz, 1H), 4.70-4.80(m, 1H), 4.93(t, J=5.4Hz, 1H), 7.05(d, J=8.9Hz, 2H), |
| | | 7.74(d, J=8.9Hz, 2H), 8.30-8.50(m, 2H), 8.92(brs, 1H), 10.95(brs, 1H). |
| 38 | A | (+)-N-Hydroxy-6-(3-methylbenzenesulfonyl)-5,6,7,8-tetrahydro- |
| | | pyrido[3,4-b]pyrazine-7-carboxamide; |
| | | ¹H-NMR(250MHz, DMSO-d₆)δ; 2.34(s, 3H), 2.94(dd, J=1.9 and 17.3Hz, 1H), |
| | | 3.14(dd, J=6.9 and 17.3Hz, 1H), 4.59(d, J=16.8Hz, 1H), 4.70(d, J=16.8Hz, |
| | | 1H), 4.77(dd, J=6.9 and 1.9Hz, 1H), 7.30-7.50(m, 2H), 7.50-7.70(m, 2H), 8.30-8.50 |
| | | (m, 2H), 8.92(brs, 1H), 10.97(brs, 1H). |
| 39 | B | (+)-N-Hydroxy-6-(4-methylthiobenzenesulfonyl)-5,6,7,8-tetra- |
| | | hydropyrido[3,4-b]pyrazine-7-carboxamide; |
| | | ¹H-NMR(250MHz, DMSO-d₆)δ; 2.50(s, 3H), 2.95(dd, J=1.7 and 17.4Hz, 1H), |
| | | 3.19(dd, J=6.8 and 17.4Hz, 1H), 4.58(d, J=16.6Hz, 1H), 4.67(d, J=16.6Hz, |
| | | 1H), 4.78(dd, J=1.7 and 6.8Hz, 1H), 7.36(d, J=8.7Hz, 2H), 7.70(d, J=8.7Hz, |
| | | 2H), 8.39(d, J=2.7Hz, 1H), 8.42(d, J=2.7Hz, 1H), 8.91(d, J=1.6Hz, 1H), 10.95 |
| | | (d, J=1.6Hz, 1H). |
| 40 | A | (+)-N-Hydroxy-6-benzenesulfonyl-5,6,7,8-tetrahydropyrido[3,4-b]- |
| | | pyrazine-7-carboxamide; |
| | | ¹H-NMR(250MHz, CDCl₃)δ; 2.80-3.00(m, 1H), 3.41(d, J=16.4Hz, 1H), 4.62 |
| | | (d, J=17.7Hz, 1H), 4.82(d, J=17.7Hz, 1H), 4.90-5.10(m, 1H), 7.30-7.60(m, |
| | | 3H), 7.78(d, J=7.6Hz, 2H), 8.20-8.40(m, 2H), 9.91(br, 1H). |
| | | MALDI-TOF MS No.11576, M.W.334.35): 335[M+H]⁺, 357[M+Na]⁺, |
| | | 373[M+K]⁺ |
| 41 | A | (+)-N-Hydroxy-6-(4-fluorobenzenesulfonyl)-5,6,7,8-tetrahydro- |
| | | pyrido[3,4-b]pyrazine-7-carboxamide; |
| | | ¹H-NMR(250MHz, CDCl₃)δ; 2.80-3.10(m, 1H), 3.37(d, J=17.8Hz, 1H), 4.62 |
| | | (d, J=17.3Hz, 1H), 4.77(d, J=17.3Hz, 1H), 4.90-5.10(m, 1H), 7.00-7.20(m, |
| | | 2H), 7.70-7.90(m, 2H), 8.10-8.40(m, 2H), 10.11(brs, 1H). |
| | | MALDI-TOF MS(M.W.352.34): 353[M+H]⁺, 375[M+Na]⁺, 391[M+K]⁺ |
| 42 | A | (+)-N-Hydroxy-6-(4-hexylbenzenesulfonyl)-5,6,7,8-tetrahydro |
| | | pyrido[3,4-b]pyrazine-7-carboxamide; |
| | | ¹H-NMR(250MHz, CDCl₃)δ; 0.80-1.00(m, 3H), 1.20-1.40(m, 6H), 1.50-1.70 |
| | | (m, 2H), 2.59(t, J=7.6Hz, 2H), 2.70-3.00(m, 1H), 3.30-3.60(m, 1H), 4.58(d, J= |
| | | 17.5Hz, 1H), 4.81(d, J=17.5Hz, 1H), 4.90-5.10(m, 1H), 7.20-7.30(m, 2H), 7.67 |
| | | (d, J=7.8Hz, 2H), 8.10-8.40(m, 2H), 9.75(brs, 1H). |
| 43 | A | (+)-N-Hydroxy-6-(4-aminobenzenesulfonyl)-5,6,7,8-tetrahydro- |
| | | pyrido[3,4-b]pyrazine-7-carboxamide; |
| | | ¹H-NMR(250MHz, DMSO-d₆)δ; 2.80-3.20(m, 2H), 4.40-4.70(m, 2H), 4.70(dd, |
| | | J=1.7 and 6.7Hz, 1H), 6.07(brs, 2H), 6.40-6.70(m, 2H), 7.30-7.50(m, 2H), |
| | | 8.30-8.50(m, 2H), 8.91(brs, 1H), 10.93(brs, 1H). |
| 44 | A | (+)-N-Hydroxy-6-(biphenyl-4-sulfonyl)-5,6,7,8-tetrahydropyrido |
| | | [3,4-b]pyrazine-7-carboxamide; |
| | | ¹H-NMR(250MHz, DMSO-d₆)δ; 2.98(d, J=17.5Hz, 1H), 3.23(dd, J=6.6 and 17.5Hz, |
| | | 1H), 4.64(d, J=16.8Hz, 1H), 4.75(d, J=16.8Hz, 1H), 4.80-4.90(m, 1H), |
| | | 7.40-7.60(m, 3H), 7.70-7.80(m, 2H), 7.80-8.00(m, 4H), 8.30-8.50(m, 2H), 8.93 |
| | | (s, 1H), 10.98(brs, 1H). |
| | | MALDI-TOF MS(M.W.410.45): 411[M+H]⁺, 433[M+Na]⁺, 449[M+K]⁺ |
| 45 | A | (+)-N-Hydroxy-6-(4-hdroxybenzenesulfonyl)-5,6,7,8-tetrahydro- |
| | | pyrido[3,4-b]pyrazine-7-carboxamide; |
| | | ¹H-NMR(250MHz, DMSO-d₆)δ; 2.94(d, J=16.9Hz, 1H), 3.00-3.30(m, 1H), 4.50-4.70 |
| | | (m, 2H), 4.70-4.80(m, 1H), 6.84(d, J=8.4Hz, 2H), 7.64(d, J=8.4Hz, |
| | | 2H), 8.30-8.50(m, 2H), 8.90(brs, 1H), 10.78(brs, 1H). |
| 46 | B | (+)-N-Hydroxy-6-(4-acetylbenzenesulfonyl)-5,6,7,8-tetrahydro- |
| | | pyrido[3,4-b]pyrazine-7-carboxamide; |
| | | ¹H-NMR(250Hz, MeOH-d₄)δ; 2.21(s, 3H), 3.00-3.30(m, 2H), 4.67(d, J=16.9Hz, |
| | | 1H), 4.80-5.10(m, 2H), 7.79(d, J=9.1Hz, 2H), 7.84(d, J=9.1Hz, 2H), 8.33(d, |
| | | J=2.5Hz, 1H), 8.38(d, J=2.5Hz, 1H). |
| 47 | A | (+)-N-Hydroxy-6-(4-propylbenzenesulfonyl)-5,6,7,8-tetrahydro- |
| | | pyrido[3,4-b]pyrazine-7-carboxamide; |
| | | ¹H-NMR(250MHz, DMSO-d₆)δ; 0.83(t, J=7.3Hz, 3H), 1.40-1.70(m, 2H), 2.59 |
| | | (t, J=7.6Hz, 2H), 2.80-3.10(m, 1H), 3.10(dd, J=7.0 and 17.2Hz, 1H), 4.60(d, J= |
| | | 17.0Hz, 1H), 4.69(d, J=17.0Hz, 1H), 4.78(dd, J=2.2 and 7.0Hz, 1H), 7.34(d, J= |
| | | 8.2Hz, 2H), 7.71(d, J=8.2Hz, 2H), 8.30-8.50(m, 2H), 8.90(brs, 1H), 10.90(br |
| | | s, 1H). |
| 48 | B | (+)-N-Hydroxy-6-(4-vinylbenzenesulfonyl)-5,6,7,8-tetrahydro- |
| | | pyrido[3,4-b]pyrazine-7-carboxamide; |
| | | ¹H-NMR(250MHz, DMSO-d₆)δ; 3.05(d, J=17.2Hz, 1H), 3.28(dd, J=6.5 and 17.2Hz, |
| | | 1H), 4.71(d, J=17.0Hz, 1H), 4.81(d, J=17.0Hz, 1H), 4.85-5.00(m, 1H), |
| | | 5.55(d, J=10.8Hz, 1H), 6.10(d, J=17.5Hz, 1H), 6.90(dd, J=10.8 and 17.5Hz, |
| | | 1H), 7.73(d, J=8.4Hz, 2H), 7.88(d, J=8.4Hz, 2H), 8.40-8.60(m, 2H), 9.02(s, 1H), |
| | | 11.04(brs, 1H). |
| 49 | B | (+)-N-Hydroxy-6-[4-(3-phenylpropoxy)benzenesulfonyl]-5,6,7,8-tetrahydropyrido |
| | | [3,4-b]pyrazine-7-carboxamide; |
| | | ¹H-NMR(250MHz, DMSO-d₆)δ; 1.90-2.20(m, 2H), 2.71(t, J=7.7Hz, 2H), 2.94 |
| | | (d, J=17.5Hz, 1H), 3.14(dd, J=7.0 and 17.5Hz, 1H), 4.01(t, J=6.3Hz, 2H), 4.58 |
| | | (d, J=16.7Hz, 1H), 4.66(d, J=16.7Hz, 1H), 4.70-4.80(m, 1H), 7.03(d, J=8.9Hz, |
| | | 2H), 7.10-7.30(m, 5H), 7.72(d, J=8.9Hz, 2H), 8.37(d, J=2.6Hz, 1H), 8.40(d, J= |
| | | 2.6Hz, 1H), 8.90(s, 1H), 10.93(s, 1H). |
| 50 | B | (+)-N-Hydroxy-6-(thiophen-2-sulfonyl)-5,6,7,8-tetrahydropyrido- |
| | | [3,4-b]pyrazine-7-carboxamide; |
| | | ¹H-NMR(250MHz, DMSO-d₆)δ; 2.98(dd, J=2.1 and 17.2Hz, 1H), 3.19(dd, J= |
| | | 7.0 and 17.2Hz, 1H), 4.60-4.80(m, 2H), 4.81(dd, J=2.1 and 7.0Hz, 1H), 7.17(d |
| | | d, J=3.8 and 5.0Hz, 1H), 7.70(dd, J=1.4 and 3.8Hz, 1H), 7.97(dd, J=1.4 and 5.0Hz, |
| | | 1H), 8.42(d, J=2.6Hz, 1H), 8.44(d, J=2.6Hz, 1H), 8.95(s, 1H), 10.99(s, |
| | | 1H). |
| | | MALDI-TOF MS(M.W.340.38): 341[M+H]⁺, 363[M+Na]⁺, 379[M+K]⁺. |
| 51 | B | (+)-N-Hydroxy-6-(2-trans-phenylethenesulfonyl)-5,6,7,8-tetrahydro- |
| | | pyrido[3,4-b]pyrazine-7-carboxamide; |
| | | ¹H-NMR(250MHz, DMSO-d₆)δ; 3.00-3.20(m, 1H), 3.43(dd, J=6.7 and 17.0Hz, |
| | | 1H), 4.62(d, J=17.0Hz, 1H), 4.65-4.80(m, 2H), 7.25(d, J=15.4Hz, 1H), 7.30-7.45 |
| | | (m, 3H), 7.50(d, J=15.4Hz, 1H), 7.60-7.70(m, 2H), 8.44(s, 2H), 8.98(s, |
| | | 1H), 10.98(s, 1H). |
| 52 | B | (+)-N-Hydroxy-6-[4-(3-methanesulfonylpropoxy)benzenesulfonyl]- |
| | | 5,6,7,8-tetrahydropyrido[3,4-b]pyrazine-7-carboxamide; |
| | | ¹H-NMR(250MHz, DMSO-d₆)δ; 2.00-2.30(m, 2H), 2.94(dd, J=1.7 and 17.5Hz, |
| | | 1H), 3.00(s, 3H), 3.16(dd, J=6.9 and 17.5Hz, 1H), 3.20-3.40(m, 2H), 4.14(t, |
| | | J=6.2Hz, 2H), 4.58(d, J=16.9Hz, 1H), 4.67(d, J=16.9Hz, 1H), 4.77(dd, |
| | | J=6.9 and 1.7Hz, 1H), 7.05(d, J=8.9Hz, 2H), 7.75(d, J=8.9Hz, 2H), 8.30-8.50(m, 2H), |
| | | 8.90(brs, 1H), 10.94(brs, 1H). |
| 53 | B | 7,8-cis-N-Hydroxy-5,8-dimethyl-6-(4-methoxybenzenesulfonyl)-5,6,7,8-tetra |
| | | hydropyrido[3,4-b]pyrazine-7-carboxamide; |
| | | ¹H-NMR(250MHz, CDCl₃)δ; 0.92(d, J=7.1Hz, 3H), 1.66(d, J=7.1Hz, 3H), 3.68 |
| | | (dq, J=3.6 and 7.1Hz, 1H), 3.83(s, 3H), 4.29(d, J=3.6Hz, 1H), 5.23(q, J=7.1Hz, |
| | | 1H), 6.87(d, J=8.9Hz, 2H), 7.18(br, 1H), 7.68(d, J=8.9Hz, 2H), 8.33(d, J= |
| | | 2.5Hz, 1H), 8.36(d, J=2.5Hz, 1H), 9.75(br, 1H). |
| 54 | B | (+)-N-Hydroxy-2,3-dimethyl-6-(4-methoxybenzenesulfonyl)-5,6,7,8- |
| | | tetrahydropyrido[3,4-b]pyrazine-7-carboxamide; |
| | | ¹H-NMR(250MHz, DMSO-d₆)δ; 2.38(s, 3H), 2.40(s, 3H), 2.80-3.10(m, 2H), |
| | | 3.82(s, 3H), 4.40-4.70(m, 2H), 4.75(dd, J=2.0 and 6.7Hz, 1H), 7.05(d, J=8.9Hz, |
| | | 2H), 7.74(d, J=8.9Hz, 2H), 8.82(brs, 1H), 10.89(brs, 1H). |
| 55 | B | (+)-N-Hydroxy-6-(5-methylthiophen-2-sulfonyl)-5,6,7,8-tetrahydro- |
| | | pyrido[3,4-b]pyrazine-7-carboxamide; |
| | | ¹H-NMR(250MHz, DMSO-d₆)δ; 2.46(d, J=0.9Hz, 3H), 2.97(dd, J=2.1 and 17.4Hz, |
| | | 1H), 3.20(dd, J=6.9 and 17.4Hz, 1H), 4.60-4.80(m, 3H), 6.89(dd, |
| | | J=0.9 and 3.7Hz, 1H), 7.50(d, J=3.7Hz, 1H), 8.42(d, J=2.6Hz, 1H), 8.44(d, J=2.6Hz, |
| | | 1H), 8.95(s, 1H), 10.96(s, 1H). |
| 56 | B | (+)-N-Hydroxy-6-[5-(2-methoxyethyl)thiophen-2-sulfonyl]-5,6,7,8- |
| | | tetrahydropyrido[3,4-b]pyrazine-7-carboxamide; |
| | | ¹H-NMR(250MHz, DMSO-d₆)δ; 2.90-3.10(m, 3H), 3.17(dd, J=6.8 and 17.4Hz, |
| | | 1H), 3.25(s, 3H), 3.49(t, J=6.0Hz, 2H), 4.60-4.85(m, 3H), 6.95(d, J=3.8Hz, |
| | | 2H), 7.52(d, J=3.8Hz, 2H), 8.40-8.50(m, 2H), 8.95(brs, 1H), 10.99(brs, 1H). |
| 57 | B | (±)-N-Hydroxy-6-(4-ethynylbenzenesulfonyl)-5,6,7,8-tetrahydro- |
| | | pyrido[3,4-b]pyrazine-7-carboxamide; |
| | | ¹H-NMR(250MHz, DMSO-d₆)δ; 2.90-3.05(m, 1H), 3.15-3.25(m, 1H), 4.50-4.60 |
| | | (m, 1H), 4.61(d, J=16.5Hz, 1H), 4.72(d, J=16.5Hz, 1H), 4.75-4.85(m, 1H), |
| | | 7.63(d, J=8.6Hz, 2H), 7.82(d, J=8.6Hz, 2H), 8.40-8.50(m, 2H), 8.92(brs, 1H), |
| | | 10.93(brs, 1H). |
| 58 | B | (+)-N-Hydroxy-6-[2-trans-(methoxyphenyl)ethenesulfonyl]-5,6,7,8- |
| | | tetrahydropyrido[3,4-b]pyrazine-7-carboxamide; |
| | | ¹H-NMR(250MHz DMSO-d₆)δ; 3.00-3.15(m, 1H), 3.30-3.50(m, 1H), 3.78(s, |
| | | 3H), 4.58(d, J=17.2Hz, 1H), 4.60-4.80(m, 2H), 6.97(d, J=8.8Hz, 2H), 7.05(d, J= |
| | | 15.4Hz, 1H), 7.43(d, J=15.4Hz, 1H), 7.59(d, J=8.8Hz, 2H), 8.40-8.50(m, 2H), |
| | | 8.96(brs, 1H), 10.95(brs, 1H). |

a) No. of examples
b) Method

### Example 59

Preparation of Tablets

Tablets are prepared as follows containing 100 mg/tablet of (+)-N-hydroxy-6-(4-methoxybenzenesulfonyl)-5,6,7,8-tetrahydropyrido[3,4-b]pyrazine-7-carboxamide[the compound (13)].

| Components | Amount |
|---|---|
| Active principle | 100 parts by weight |
| Cornstarch | 46 parts by weight |
| Microcrystalline cellulose | 98 parts by weight |
| Hydroxypropylcellulose | 2 parts by weight |
| Magnesium Stearate | 4 parts by weight |

Active principle, cornstarch and microcrystalline cellulose are admixed and to this mixture is added hydroxypropylcellulose dissolved in 50 parts by weight of water followed by sufficient kneading. The paste is then passed through a sieve to granulate, dried, mixed with magnesium stearate and made into tablets of 250 mg each.

### Example 60

Preparation of Granules

Granules are obtained as follows containing (+)-N-hydroxy-6-(4-methoxy benzenesulfonyl)-5,6,7,8-tetrahydropyrido[3,4-b]pyrazine-7-carboxamide[the compound (13)].

| Components | Amount |
|---|---|
| Active principle | 200 parts by weight |
| Lactose | 185 parts by weight |
| Cornstarch | 109 parts by weight |
| Hydroxypropylcellulose | 6 parts by weight |

Active principle, lactose and cornstarch are admixed and to this added hydroxypropylcellulose dissolved in 120 parts by weight of water, followed by sufficient kneading. The paste is passed through a 20 mesh sieve to granulate, dried and size is adjusted to obtain granules containing 200 mg of the active principle per 500 mg of granule.

### Example 61

Preparation of Capsules

Capsules are prepared as follows containing 100 mg of (+)-N-hydroxy-6-(4-methoxybenzenesulfonyl)-5,6,7,8-tetrahydropyrido[3,4-b]pyrazine-7-carboxamide[the compound (13)] per capsule.

| Components | Amount |
|---|---|
| Active principle | 100 parts by weight |
| Lactose | 35 parts by weight |
| Cornstarch | 60 parts by weight |
| Magnesium stearate | 5 parts by weight |

The above components are mixed together and 200 mg each of this mixed powder is encapsulated to obtain capsules.

### Example 62

Preparation of Injections

A mixture of 0.5 parts by weight of (+)-N-hydroxy-6-(4-methoxybenzene sulfonyl)-5,6,7,8-tetrahydropyrido[3,4-b]pyrazine-7-carboxamide[the compound (i3)] and 5 parts by weight of sorbitol are dissolved in distilled water for injection to obtain 100 parts by weight of the solution. The solution is filtered through membrane filter and 5 g each of the filtrate is poured into an ample substituted by nitrogen gas. The ample are sealed and then sterilized by heating at 120° C. for 15 minutes to obtain injection containing 25 mg of the compound (13) per ample.

Experiment 63

Preparation of Ointment

1.0 parts by weight of (+)-N-hydroxy-6-(4-methoxybenzenesulfonyl)-5,6,7,8-tetrahydropyrido[3,4-b]pyrazine-7-carboxamide[the compound (13)] and 0.1 parts by weight of butyl paraben are dispersed in 5.0 parts by weight of light liquid paraffin.

The mixture is milled in a mortar and sieved through a 200 mesh screen. This product is mixed with 5.0 parts by weight of liquid paraffin and the mixture is mixed with 88.9 parts by weight of gelled hydrocarbon warmed at about 60° C. to take a homogenous dispersion whereupon oily ointment is obtained.

### Reference Example 1

(±)-5,6,7,8-Tetrahydropyrido[3,4-b]pyrazine-7-Carboxylic Acid Hydrochloride

(a) 2,3-Bischloromethylpyrazine

2,3-Dimethylpyrazine (75 g) was dissolved in carbon tetrachloride (750 ml) and thereto were added N-chlorosuccinimide (205 g) and benzoyl peroxide (3.0 g) and the mixture was refluxed for 20 hours. After removing the precipitated solid materials, the solvent was distilled off, and the residue was purified by a silica gel column chromatography (mobile phase;

hexane/ethyl acetate=5/1) to give the title compound (62 g) as pale brown oil.

¹H-NMR (250 MHz, CDCl₃) δ; 4.86 (4H, s), 8.54 (2H, s).

(b) Ethyl (±)-6-acetyl-7-ethoxycarbonyl-5,6,7,8-tetrahydropyrido[3,4-b]pyrazine-7-carboxylate;

2,3-Bischloromethylpyrazine (34 g) and diethyl acetamidomalonate (42 g) were dissolved in DMF (400 ml), and thereto was added 60% sodium hydride (17 g) under ice-cooling, and the mixture was stirred for 3 hours. The reaction mixture was neutralized with 1N hydrochloric acid, and the solvent was distilled off. To the residue was added ethyl acetate (900 ml), and the undissolved materials were removed. The solvent was distilled off from the solution and the residue was purified by a silica gel column chromatography (mobile phase; hexane/ethyl acetate=1/1-1/4) to give the title compound (21 g) as brown oil.

¹H-NMR (250 MHz, CDCl₃) δ; 1.21 (6H, t, J=7.1 Hz), 2.31 (3H, s), 3.70 (2H, s), 4.1-4.4 (4H, m), 4.85 (2H, s), 8.44 (1H, d, J=2.6 Hz), 8.46 (1H, d, J=2.6 Hz).

(c) (±)-5,6,7,8-Tetrahydropyrido[3,4-b]pyrazine-7-Carboxylic Acid Hydrochloride

To ethyl (±)-6-acetyl-7-ethoxycarbonyl-5,6,7,8-tetrahydropyrido[3,4-b]pyrazine-7-carboxylate (23 g) was added 6N hydrochloric acid (100 ml) and the mixture was stirred at 100° C. for 3 hours. The solvent was distilled off to give the title compound (13 g).

¹H-NMR (250 MHz, DMSO-d₆) δ; 3.3-3.5 (2H, m), 4.3-4.6 (2H, m), 4.6-4.7 (1H, m), 8.5-8.7 (2H, m).

### Reference Example 2

(±)-6-tert-Butoxycarbonyl-5,6,7,8-tetrahydropyrido[3,4-b]pyrazine-7-carboxylic Acid

(±)-5,6,7,8-Tetrahydropyrido[3,4-b]pyrazine-7-carboxylic acid hydrochloride (12 g) was dissolved in a mixture of dioxane (100 ml) and water (100 ml), and thereto was added a 1N sodium hydroxide solution (110 ml). To the mixture was added di-tert-butyl dicarbonate (13 g) under ice-cooling, and the mixture was stirred for one hour and thereafter stirred at room temperature for 3 hours. The reaction mixture was adjusted to pH 3 with a diluted hydrochloric acid and extracted with ethyl acetate. The organic layer was washed with water and a saturated saline solution and the dried over magnesium sulfate. After removing magnesium sulfate, the solvent was distilled off to give the title compound (10 g) as a brown solid.

¹H-NMR (250 MHz, CDCl₃) δ; 1.52 (9H, s), 3.34 (1H, dd, J=17.2 Hz, 6.7 Hz), 3.53 (1H, d, J=17.2 Hz), 4.5-4.7 (1H, m), 4.9-5.1 (1H, m), 5.1-5.5 (1H, m), 8.4-8.5 (2H, m).

### Reference Example 3

(−)-6-tert-Butoxycarbonyl-5,6,7,8-tetrahydropyrido[3,4-b]pyrazine-7-carboxylic Acid

(±)-N-Tert-butoxycarbonyl-5,6,7,8-Tetrahydropyrido[3,4-b]pyrazine-7-carboxylic acid obtained (35 g) was dissolved in ethyl acetate (900 ml), and thereto was added (−)-α-methyl-benzylamine (16 ml), and the mixture was allowed to stand at room temperature overnight. The precipitated solid material was taken by filtration, and dissolved in ethyl acetate (1000 ml) with heating. The mixture was allowed to stand at room temperature overnight. The precipitated solid material was separated and acidified with a 10% aqueous citric acid solution and extracted with ethyl acetate. The organic layer was washed with water and a saturated saline solution and dried over magnesium sulfate. After removing magnesium sulfate, the solvent was distilled off to give the title compound (9.0 g).

¹H-NMR (250 MHz, CDCl₃) δ; 1.52 (9H, s), 3.34 (1H, dd, J=17.2 Hz, 6.7 Hz), 3.53 (1H, d, J=17.2 Hz), 4.5-4.7 (1H, m), 4.9-5.1 (1H, m), 5.1-5.5 (1H, m), 8.4-8.5 (2H, m).

[α]_{D}; −38° (c=1.0, methanol)

### Reference Example 4

(±)-N-Benzyloxy-N-tert-butoxycarbonyl-5,6,7,8-tetrahydropyrido[3,4-b]pyrazine-7-carboxylic acid

(±)-N-Tert-butoxycarbonyl-5,6,7,8-tetrahydropyrido[3,4-b]pyrazine-7-carboxylic acid (16.7 g) was dissolved in DMF (200 ml) and thereto were added WSC (14.9 g) and HOBt (11.9 g) under ice-cooling and the mixture was stirred for one hour. To the mixture were added O-benzylhydroxylamine hydrochloride (12.4 g) and triethylamine (7.9 g), and the mixture was stirred at room temperature overnight. After distilling off the solvent, to the residue were added ethyl acetate and water, and the organic layer was washed with an aqueous sodium hydrogen carbonate solution, a diluted hydrochloric acid, water and a saturated saline solution, and dried over magnesium sulfate. After removing magnesium sulfate, the solvent was distilled off to give the title compound (21.7 g).

¹H-NMR (250 MHz, CDCl₃) δ; 1.47 (9H, s), 3.20 (1H, dd, J=6.3 Hz, 17.5 Hz), 3.42 (1H, d, J=17.5 Hz), 4.36 (1H, d, J=17.8 Hz), 4.7-5.1 (4H, m), 7.34 (5H, s), 8.3-8.5 (2H, m), 8.92 (1H, bs).

### Reference Example 5

(+)-N-Benzyloxy-6-tert-butoxycarbonyl-5,6,7,8-tetrahydropyrido[3,4-b]pyrazine-7-carboxylic Acid

(−)-6-tert-Butoxycarbonyl-5,6,7,8-tetrahydropyrido[3,4-b]pyrazine-7-carboxylic acid was treated in the same way as described in Reference Example 4 to give the title compound.

¹H-NMR (250 MHz, CDCl₃) δ; 1.47 (9H, s), 3.20 (1H, dd, J=6.3 Hz, 17.5 Hz), 3.42 (1H, d, J=17.5 Hz), 4.36 (1H, d, J=17.8 Hz), 4.7-5.1 (4H, m), 7.34 (5H, s), 8.3-8.5 (2H, m), 8.92 (1H, bs).

### Reference Example 6

(±)-N-Benzyloxy-5,6,7,8-tetrahydropyrido[3,4-b]pyrazine-7-carboxamide Hydrochloride

To (±)-N-Benzyloxy-6-tert-butoxycarbonyl-5,6,7,8-tetrahydropyrido[3,4-b]pyrazine-7-carboxamide (22 g) was dissolved in ethyl acetate (30 ml), and thereto was further added a 4N hydrochloric acid/ethyl acetate (150 ml) under ice-cooling and the mixture was stirred for two hours. The precipitated solid material was taken by filtration to give the title compound (18 g).

¹H-NMR (250 MHz, DMSO-d₆) δ; 3.19 (1H, dd, J=11.4 Hz, 17.7 Hz), 3.36 (1H, dd, J=5.2 Hz, 17.7 Hz), 4.2-4.4 (2H, m), 4.50 (1H, d, J=16.6 Hz), 4.86 (1H, d, J=11.1 Hz), 4.91 (1H, d, J=11.1 Hz), 7.3-7.5 (5H, m), 8.5-8.6 (2H, m), 9.9-10.7 (1H, br), 12.2 (1H, bs).

### Reference Example 7

(+)-N-Benzyloxy-5,6,7,8-tetrahydropyrido[3,4-b]pyrazine-7-carboxylic Acid Hydrochloride

(±)-N-Benzyloxy-6-tert-butoxycarbonyl-5,6,7,8-tetrahydropyrido[3,4-b]-pyrazine-7-carboxamide was treated in the same way as described in Reference Example 6 to give the title compound.

¹H-NMR (250 MHz, DMSO-d₆) δ; 3.19 (1H, dd, J=11.4 Hz, 17.7 Hz), 3.36 (1H, dd, J=5.2 Hz, 17.7 Hz), 4.2-4.4 (2H, m), 4.50 (1H, d, J=16.6 Hz), 4.86 (1H, d, J=11.1 Hz), 4.91 (1H, d, J=11.1 Hz), 7.3-7.5 (5H, m), 8.5-8.6 (2H, m), 9.9-10.7 (1H, br), 12.2 (1H, bs).

[α]_{D}; +88° (c=1.0, methanol)

### Reference Example 8

Methyl (i)-5,6,7,8-tetrahydropyrido[3,4-b]pyrazine-7-carboxylate Hydrochloride

To Ethyl (±)-6-acetyl-7-ethoxycarbonyl-5,6,7,8-tetrahydropyrido [3,4-b]pyrazine-7-carboxylate (3.6 g) was added a 6N hydrochloric acid (40 ml) and the mixture was refluxed for one hour. After distilling off the solvent, to the residue was added methanol (30 ml) and further added dropwise thionyl chloride (3.0 ml) under ice-cooling, and the mixture was refluxed for 5 hours. The solvent was distilled off to give the title compound (2.9 g).

¹H-NMR (250 MHz, DMSO-d₆) δ; 3.3-3.5 (2H, m), 3.85 (3H, s), 4.3-4.5 (2H, m), 4.6-4.7 (1H, m), 8.5-8.7 (2H, m).

### Reference Example 9

(±)-5,6,7,8-Tetrahydropyrido[3,4-b]pyrazine-7-carboxylic Acid Tert-Butyl Ester

(a) (±)-6-Benzyloxycarbonyl-5,6,7,8-tetrahydropyrido[3,4-b]pyrazine-7-carboxylic Acid

To Ethyl (±)-6-acetyl-7-ethoxycarbonyl-5,6,7,8-tetrahydropyrido[3,4-b]pyrazine-7-carboxylate (21 g) was added 6N hydrochloric acid (100 ml), and the mixture was refluxed for 3 hours. After distilling off the solvent, to the residue were added dioxane (200 ml) and water (300 ml), and thereto was further added sodium carbonate (21 g). To the mixture was added a solution of Z-chloride (12 g) in dioxane (100 ml), and the mixture was stirred at room temperature for 16 hours. After distilling off the solvent, the residue was acidified with a diluted hydrochloric acid and the mixture was extracted with ethyl acetate. The organic layer was washed with water and a saturated saline solution and dried over magnesium sulfate. The solvent was distilled off to give the title compound (16 g).

¹H-NMR (250 MHz, CDCl₃) δ; 3.38 (1H, dd, J=17.5 Hz, 6.5 Hz), 3.5-3.6 (1H, m), 4.68 (1H, d, J=18.0 Hz), 5.05 (1H, d, J=18.0 Hz), 5.1-5.4 (2H, m), 5.4-5.5 (1H, m), 7.3-7.4 (5H, m), 8.4-8.5 (2H, m).

(b) Tert-butyl (±)-N-benzyloxycarbonyl-5,6,7,8-tetrahydropyrido[3,4-b]pyrazine-7-carboxylate

To (±)-N-benzyloxycarbonyl-5,6,7,8-tetrahydropyrido[3,4-b]pyrazine-7-carboxylic acid (16 g) was added dichloromethane (100 ml) and thereto was blown isobutene (28 g) at −78° C. To the mixture was added conc. sulfuric acid (1.0 ml) and the mixture was stirred at room temperature for 7 days. After distilling off the solvent, the residue was neutralized with an aqueous sodium hydrogen carbonate solution and extracted with ethyl acetate. The organic layer was washed with water and a saturated saline solution, and dried over magnesium sulfate. After distilling off the solvent, the residue was purified by silica gel column chromatography (mobile phase; hexane/ethyl acetate=1/1) to give the title compound (7.1 g).

¹H-NMR (250 MHz, CDCl₃) δ; 1.32 (9H, s), 3.32 (1H, dd, J=16.9 Hz, 5.9 Hz), 3.4-3.6 (1H, m), 4.6-4.8 (1H, m), 4.9-5.1 (1H, m), 5.1-5.4 (3H, m), 7.3-7.5 (5H, m), 8.43 (2H, s).

(c) Tert-butyl (±)-5,6,7,8-tetrahydropyrido[3,4-b]pyrazine-7-carboxylate

Tert-butyl (±)-N-benzyloxycarbonyl-5,6,7,8-tetrahydropyrido[3,4-b]pyrazine-7-carboxylate (2.7 g) was dissolved in methanol (30 ml) and the mixture was treated with 10% Pd—C (0.27 g) with stirring under hydrogen gas atmosphere at room temperature for one hour. After removing Pd—C, the solvent was distilled off to give the title compound (1.49 g).

¹H-NMR (250 MHz, CDCl₃) δ; 1.50 (9H, s), 3.15 (1H, dd, J=17.2 Hz, 9.3 Hz), 3.29 (1H, dd, J=17.2 Hz, 4.9 Hz), 3.77 (1H, dd, J=9.3 Hz, 4.9 Hz), 4.15 (1H, d, J=17.0 Hz), 4.27 (1H, d, J=17.0 Hz), 8.3-8.4 (2H, m).

### Reference Example 10

(+)-5,6,7,8-Tetrahydropyrido[3,4-b]pyrazine-7-carboxylic Acid Hydrochloride

To (−)-6-tert-Butoxycarbonyl-5,6,7,8-tetrahydropyrido[3,4-b]pyrazine-7-carboxylic acid (2.0 g) was added 4N hydrochloric acid/ethyl acetate (15 ml) under ice-cooling and the mixture was stirred at room temperature for 4 hours. The precipitated solid was collected by filtration to give the title compound (1.5 g).

¹H-NMR (250 MHz, CDCl₃) δ; 3.3-3.5 (2H, m), 4.3-4.6 (2H, m), 4.6-4.7 (1H, m), 8.5-8.7 (2H, m)

[α]_{D}; +79° (c=0.51, methanol)

### Reference Example 11

(Pyrazine-2-yloxy)benzene

Phenol (11.9 g) was dissolved in DMF (150 ml) and thereto was added 60% sodium hydride (5.0 g) under ice-cooling and the mixture was stirred at room temperature for 30 minutes.

To the mixture was added 2-chloropyrazine (14.4 g), and the mixture was stirred at 80° C. overnight. To the mixture was added water (150 ml) and the mixture was extracted with ethyl acetate. The organic layer was washed with water and a saturated saline solution, and dried over magnesium sulfate. After removing magnesium sulfate, the solvent was distilled off to give the title compound (22.4 g) as yellow powders.

¹H-NMR (250 MHz, CDCl₃) δ; 7.1-7.3 (3H, m), 7.3-7.5 (2H, m), 8.10 (1H, d, J=2.6 Hz), 8.26 (1H, d, J=2.6 Hz), 8.42 (1H, s).

### Reference Example 12

4-Nitrophenoxybenzene

Phenol and 4-bromonitrobenzene were treated in the same way as described in Reference Example 11 to give the title compound.

¹H-NMR (250 MHz, CDCl₃) δ; 7.0-7.1 (2H, m), 7.1-7.2 (2H, m), 7.2-7.3 (1H, m), 7.3-7.5 (2H, m), 8.1-8.3 (2H, m)

### Reference Example 13

Preparation of Phenoxyether

A typical example is shown as follows by the preparation of 3-phenoxypropanol.

Phenol (13.6 g) was dissolved in DMF (300 ml) and thereto were added 60% sodium hydride (6.0 g) and 3-bromopropanol (20 g) under ice-cooling and the mixture was stirred at room temperature overnight. After distilling off the solvent, to the mixture was added water (300 ml) and the mixture was extracted with ethyl acetate. The organic layer was washed with a 1N hydrochloric acid, water, an aqueous sodium hydrogen carbonate solution, water and a saturated saline solution, and dried over magnesium sulfate. After removing magnesium sulfate, the solvent was distilled off, and the residue was purified by a silica gel column chromatography (mobile phase; ethyl acetate/n-hexane=1/2) to give the title compound (20.4 g) as colorless oil. Physical constant was shown in the following table.

The following phenoxyethers were prepared according to the example shown above.

TABLE 5

| No. | Obtained compounds and their physical constants |
|---|---|
| 13-1 | 3-Phenoxypropanol |
| | ¹H-NMR(250MHz, CDCl₃)δ; 1.95(s, 1H), 2.03(tt, J=6.0 and 5.9Hz, 2H), 3.85(t, J= |
| | 5.9Hz, 2H), 4.11(t, J=6.0Hz, 2H), 6.80-7.00(m, 3H), 7.20-7.40(m, 2H). |
| 13-2 | Propoxybenzene |
| | ¹H-NMR(250MHz, CDCl₃)δ; 1.04(t J=7.4Hz, 3H), 1.70-2.05(m, 2H), 3.92(t, J=6.6Hz, |
| | 2H), 6.85-7.00(m, 3H), 7.20-7.35(m, 2H). |
| 13-3 | Isobutoxybenzene |
| | ¹H-NMR(250MHz, CDCl₃)δ; 1.02(d, J=6.7Hz, 6H), 2.00-2.20(m, 1H), 3.72(d, J=6.6Hz, |
| | 2H), 6.80-7.00(m, 3H), 7.20-7.40(m, 2H). |
| 13-4 | Ethyl phenoxyacetate |
| | ¹H-NMR(250MHz, CDCl₃)δ; 1.31(t, J=7.1Hz, 3H), 4.29(q, J=7.1Hz, 2H), 4.64(s, |
| | 2H), 6.90-7.10(m, 3H), 7.20-7.40(m, 2H). |
| 13-5 | Ethyl 4-phenoxybutanoate |
| | ¹H-NMR(250MHz, CDCl₃)δ; 1.28(t, J=7.1Hz, 3H), 2.00-2.20(m, 2H), 2.54(t, J=7.3Hz, |
| | 2H), 4.05(t, J=6.1Hz, 2H), 4.16(q, J=7.1Hz, 2H), 6.80-7.00(m, 3H), 7.20-7.40 |
| | (m, 2H). |
| 13-6 | 2-Phenoxyethyl acetate |
| | ¹H-NMR(250MHz, CDCl₃)δ; 2.12(s, 3H), 4.10-4.30(m, 2H), 4.40-4.50(m, 2H), 6.90-7.00 |
| | (m, 3H), 7.20-7.40(m, 2H) |

### Reference Example 14

Preparation of 3-phenoxypropylether

A typical example is shown as follows by the preparation of (3-butoxypropoxy)benzene

To the DMF-solution (35 ml) of 3-phenoxypropanol (4.02 g), were added 60% sodium hydride (1.67 g) and 1-iodobutane (5.5 ml), and the mixture was stirred at room temperature for 60 hours and then at 60° C. for 8 hours. After ice water was added to the reaction mixture, the whole solution was extracted by ethyl acetate. The ethyl acetate layer was washed by 1N hydrochloric acid, water, saturated sodium bicarbonate solution, water and saturated sodium chloride solution successively and dried over magnesium sulfate. After removing magnesium sulfate, solvent was distilled off and the residue was purified by a silica gel column chromatography of medium pressure (mobile phase; n-hexane/ethyl acetate=4/1) to give the title compound (3.42 g) as colorless oil.

The following 3-phenoxypropylethers were prepared according to the example shown above.

TABLE 6

| No. | Obtained compounds and their physical constants |
|---|---|
| 14-1 | 3-Butoxypropoxybenzene |
| | ¹H-NMR(250MHz, CDCl₃)δ; 0.91(t, J=7.3Hz, 3H), 1.20-1.45(m, 2H), 1.45-1.65 |
| | (m, 2H), 1.90-2.10(m, 2H), 3.43(t, J=6.6Hz, 2H), 3.59(t, J=6.2Hz, 2H), 4.06(t, J=6.3Hz, |
| | 2H), 6.80-7.00(m, 3H), 7.20-7.35(m, 2H). |
| 14-2 | 3-Methoxypropoxybenzene |
| | ¹H-NMR(250MHz, CDCl₃)δ; 2.04(tt, J=6.3 and 6.2Hz, 2H), 3.35(s, 3H), 3.56(t, J= |
| | 6.2Hz, 2H), 4.05(t, J=6.3Hz, 2H), 6.80-7.00(m, 3H), 7.20-7.40(m, 2H). |
| 14-3 | 3-Ethoxypropoxybenzene |
| | ¹H-NMR(250MHz, CDCl₃)δ; 1.21(t, J=7.0Hz, 3H), 2.05(q, J=6.3Hz, 2H), 3.50(q, J= |
| | 7.0Hz, 2H), 3.61(t, J=6.3Hz, 2H), 4.07(t, J=6.3Hz, 2H), 6.90-7.00(m, 3H), 7.20-7.30 |
| | (m, 2H). |
| 14-4 | (3-Isobutoxypropoxy)benzene |
| | ¹H-NMR(250MHz, CDCl₃)δ; 0.89(d, J=6.7Hz, 6H), 1.70-2.10(m, 3H), 3.19(d, J= |
| | 6.7Hz, 2H), 3.59(t, J=6.2Hz, 2H), 4.07(t, J=6.3Hz, 2H), 6.80-7.00(m, 3H), 7.20-7.40 |
| | (m, 2H). |

### Reference Example 15

(4-Methoxybutoxy)benzene

(1) 4-Phenoxybutanol;

To a solution of phenol (10.0 g) in DMF (50 ml) was added in portions 60% sodium hydride (4.3 g) under ice-cooling and the mixture was stirred for 10 minutes. To the mixture was added 4-chlorobutanol (9.6 g) under ice-cooling and the mixture was stirred at room temperature for 14 hours and further at 60° C. for 22 hours. The reaction mixture was poured into water (500 ml) and the mixture was extracted with ethyl acetate. The ethyl acetate layer was washed with a 1N aqueous sodium hydroxide solution and water, and dried over magnesium sulfate. The solvent was distilled off under reduced pressure to give pale red solid material. This material was purified by a silica gel column chromatography (solvent; cyclohexane/ethyl acetate=1/1) to give the title compound (1.80 g) as colorless oil.

¹H-NMR (CDCl₃) δ; 1.6-2.0 (4H, m), 3.74 (2H, t, J=6.2 Hz), 4.03 (2H, t, J=6.0 Hz), 6.8-7.0 (3H, m), 7.2-7.4 (2H, m).

(2) (4-Methoxybutoxy)benzene;

To a solution of 4-phenoxybutanol (1.80 g) in DMF (10 ml) was added in portions 60% sodium hydride (0.65 g) at room temperature and the mixture was stirred at 50° C. for 5 minutes. To the mixture was added methyl iodide (7.66 g) under ice-cooling and the mixture was further stirred at room temperature for 15 hours. The reaction mixture was poured into water (500 ml) and the mixture was extracted with ethyl acetate. The ethyl acetate layer was washed with water, dried over magnesium sulfate, and distilled the solvent under reduced pressure. Finally, the residue was purified by a silica gel column chromatography (solvent; cyclohexane/ethyl acetate=1/1) to give the title compound (1.40 g) as colorless oil.

¹H-NMR (CDCl₃) δ; 1.7-2.0 (4H, m), 3.36 (3H, s), 3.46 (2H, t, J=6.2 Hz), 4.0 (2H, t, J=6.2 Hz), 6.8-7.0 (3H, m), 7.2-7.4 (2H, m).

### Reference Example 16

Preparation of 2-alkoxyethoxybenzene

A typical example is shown as follows by the preparation of 2-methoxyethoxybenzene

Phenol (10 g) was dissolved in DMF (150 ml) and thereto were added 60% sodium hydride (4.8 g) and 2-chloroethyl methyl ether (10 g) under ice-cooling and the mixture was stirred at room temperature overnight. To the mixture was added water and the mixture was extracted with ethyl acetate. The organic layer was washed with an aqueous sodium hydrogen carbonate solution, water and a saturated saline solution, and dried over magnesium sulfate.

After removing magnesium sulfate, the residue was purified by a silica gel column chromatography (mobile phase; ethyl acetate/n-hexane=1/4) to give the title compound (12.5 g) as colorless oil.

The following 2-alkoxyethoxybenzenes were prepared according to the example shown above.

TABLE 7

| No. | Obtained compounds and their physical constants |
|---|---|
| 16-1 | 2-Methoxyethoxybenzene |
| | ¹H-NMR(250MHz, CDCl₃)δ; 3.46(s, 3H), |
| | 3.70-3.80(m, 2H), 4.10-4.20(m, 2H), 6.80-7.00 |
| | (m, 3H), 7.20-7.40(m, 2H). |
| 16-2 | 2-Ethoxyethoxybenzene |
| | ¹H-NMR(250MHz, CDCl₃)δ; 1.25(t, J=7.0Hz, 3H), |
| | 3.61(q, J=7.0Hz, 2H), 3.80(t, J= |
| | 4.9Hz, 2H), 4.13(t, J=4.9Hz, 2H), 6.90-7.00(m, 3H), |
| | 7.20-7.40(m, 2H). |

### Reference Example 17

Preparation of Alkoxybenzene(1)

A typical example is shown as follows by the preparation of 3-methylthiopropoxybenzene.

After 3-methylthiopropanol (5 g) was dissolved in THF (50 ml), triethylamine (5.3 g) and methanesulfonylchloride (5.9 g) were added, and the mixture was stirred for 3.5 hours. After it was acidified by diluted hydrochloric acid, the solution was extracted with ethyl acetate. The organic layer was washed by water and saturated sodium chloride aqueous solution, and dried over magnesium sulfate. After removing magnesium sulfate, solvent was distilled off to give 3-methylthiopropyl methanesulfonate (8.1 g) as colorless oil.

Next, phenol (4.1 g) was dissolved in DMF (10 ml), 60% sodium hydride (1.9 g) and 3-methylthiopropyl methanesulfonate (8.1 g) were added and the mixture was stirred at room temperature overnight. After water was added to the reaction mixture, it was extracted by ethyl acetate and the organic layer was washed with aqueous sodium bicarbonate solution, water and saturated sodium chloride solution, and dried over magnesium sulfate. After magnesium sulfate was removed, the solvent was distilled off and the resulting residue was purified by a silica gel column chromatography of medium pressure (mobile phase; ethyl acetate/n-hexane=1/4) to give the title compound (7.8 g) as colorless oil.

In the same manner, pentyloxybenzene was prepared. Physical constants of the obtained compounds were shown below.

TABLE 8

| No. | Physical constants of the obtained compounds |
|---|---|
| 17-1 | 3-Methylthiopropoxybenzene |
| | ¹H-NMR(250MHz, CDCl₃)δ; 2.00-2.20(m, 2H), |
| | 2.13(s, 3H), 2.70(t, J=7.2Hz, 2H), |
| | 4.08(t, J=6.1Hz, 2H), 6.80-7.00(m, 3H), 7.20-7.40(m, 2H). |
| 17-2 | Pentyloxybenzene |
| | ¹H-NMR(250MHz, CDCl₃)δ; |
| | 0.95(t, J=7.1Hz, 3H), 1.30-1.60(m, 4H), 1.70-1.90 |
| | (m, 2H), 3.97(t, J=6.6Hz, 2H), |
| | 6.80-7.00(m, 3H), 7.20-7.40(m, 2H). |

### Reference Example 18

Preparation of Alkoxybenzene(2)

A typical example is shown as follows by the preparation of 2-fluoroethoxybenzene.

Phenol (0.74 g) was dissolved in acetone (50 ml) and thereto were added 1-bromo-2-fluoroethane (1.0 g) and potassium carbonate (2.2 g) and the mixture was stirred at room temperature for 2 days and thereafter refluxed for 8 hours. After distilling off the solvent, ethyl acetate (50 ml) was added to the mixture, and the resulting mixture was washed with a 1N sodium hydroxide solution (50 ml) four times and dried over sodium sulfate. After removing sodium sulfate, the solvent was distilled off to give the title compound (0.88 g). In the same manner, 3-phenoxypropionitrile was prepared from phenol and 3-bromopropionitrile. Physical constants of the obtained compounds were shown below.

TABLE 9

| No. | Physical constants of the obtained compounds |
|---|---|
| 18-1 | 2-Fluoroethoxybenzene |
| | ¹H-NMR(250MHz, CDCl₃)δ; |
| | 4.10-4.30(m, 2H), 4.60-4.90(m, 2H), 6.90-7.00(m, |
| | 3H), 7.20-7.40(m, 2H). |
| 18-2 | 3-Phenoxypropionitrole |
| | ¹H-NMR(250MHz, CDCl₃)δ; |
| | 2.83(t, J=6.4Hz, 2H), 4.21(t, J=6.4Hz, 2H), 6.80-7.10 |
| | (m, 3H), 7.20-7.40(m, 2H). |

### Reference Example 19

(3-Methanesulfonylpropoxy)benzene

To the mixture of 3-methylthiopropoxybenzene (3.72 g) and acetic acid (100 ml), was added 30% aqueous hydrogen peroxide solution and the solution was stirred at room temperature for 2 days. After ice was added to the reaction mixture, the precipitated solid was collected by filtration and washed with water to give the title compound (2.18 g).

¹H-NMR(250 MHz, CDCl₃) δ; 2.2-2.5 (m, 2H), 2.96 (s, 3H), 3.2-3.4 (m, 2H), 4.12 (t, J=5.8 Hz, 2H), 6.8-7.1 (m, 3H), 7.2-7.4 (m, 2H).

### Reference Example 20

2-(2-Methoxyethyl)thiophen

2-(Thiophen-2-yl)ethanol (5 g, 0.039 mole) was dissolved in DMF (100 ml) and thereto was added 60% sodium hydride (1.8 g, 0.045 mole), and the mixture was stirred at room temperature for half an hour. Then methyl iodide (8.1 g, 0.057 mole) was added and the mixture was stirred again at room temperature for half an hour. To the reaction mixture ice was added and it was extracted with ethyl acetate. The organic layer was washed three times with water and once with saturated sodium chloride solution, and dried over magnesium sulfate. After removing magnesium sulfate, the solvent was distilled off to give the title compound (5.1 g) as oil.

¹H-NMR(250 MHz, CDCl₃) δ; 3.12 (dt, J=0.7 and 6.7 Hz, 2H), 3.41 (s, 3H), 3.65 (t, J=6.7 Hz, 2H), 6.85-6.90 (m, 1H), 6.96 (dd, J=2.3 and 5.5 Hz, 1H), 7.16 (dd, J=1.2 and 5.5 Hz, 1H).

### Reference Example 21

Preparation of sulfonylchloride(1)

A typical example is shown as follows by the preparation of 4-(3-methoxypropoxy)benzenesulfonyl chloride;

3-Methoxypropoxybenzene (4.7 g) was dissolved in 1,2-dichloroethane (20 ml) and thereto was added chlorosulfonic acid (5.7 ml) under ice-cooling, and the mixture was stirred for one hour. The reaction mixture was poured into ice-water (300 ml) and the mixture was extracted with chloroform. The organic layer was washed with a 1N hydrochloric acid and a saturated saline solution and dried over magnesium sulfate. After removing magnesium sulfate, the solvent was distilled off to give the title compound (2.6 g) as colorless oil.

The sulfonylchlorides below were prepared according to the example shown above.

TABLE 10

| No. | Physical constants of the obtained compounds |
|---|---|
| 21-1 | 4-(3-Methoxypropoxy)benzenesulfonylchloride |
| | ¹H-NMR(250MHz, CDCl₃)δ; 2.09(tt, J=6.2 |
| | and6.1Hz, 2H), 3.36(s, 3H), 3.56(t, J= |
| | 6.1Hz, 2H), 4.18(t, J=6.2Hz, 2H), 7.04(d, J=9.1Hz, 2H), |
| | 7.96(d, J=9.1Hz, 2H). |
| 21-2 | 4-(Pyrazine-2-yloxy)benzenesulfonylchloride |
| | ¹H-NMR(250MHz, CDCl₃)δ; 7.42(d, |
| | J=8.9Hz, 2H), 8.10(d, J=8.9Hz, 2H), 8.17(d, |
| | J=2.5Hz, 1H), 8.41(d, J=2.5Hz, 1H), 8.55(s, 1H). |
| 21-3 | 4-(2-Ethoxyethoxy)benzenesulfonylchloride |
| | ¹H-NMR(250MHz, CDCl₃)δ; |
| | 1.24(t, J=7.0Hz, 3H), 3.63(q, J=7.0Hz, 2H), |
| | 3.70-3.90 |
| | (m, 2H), 4.20-4.30(m, 2H), 7.06(d, J=9.0Hz, |
| | 2H), 7.96(d, J=9.0Hz, 2H). |
| 21-4 | 4-(2-Methoxyethoxy)benzenesulfonylchloride |
| | ¹H-NMR(250MHz, CDCl₃)δ; |
| | 3.45(s, 3H), 3.70-3.80(m, 2H), 4.20-4.30(m, 2H), 7.07 |
| | (d, J=9.0Hz, 2H), 7.95(d, J=9.0Hz, 2H). |
| 21-5 | 4-Pentyloxybenzenesulfonylchloride |
| | ¹H-NMR(250MHz, CDCl₃)δ; |
| | 0.96(t, J=7.0Hz, 3H), 1.30-1.60(m, 4H), 1.70-2.00 |
| | (m, 2H), 4.08(t, J=6.5Hz, 2H), 7.04(d, J=9.1Hz, |
| | 2H), 7.98(d, J=9.1Hz, 2H). |
| 21-6 | 4-(3-Methylthiopropoxy)benzenesulfonylchloride |
| | ¹H-NMR(250MHz, CDCl₃)δ; |
| | 2.14(s, 3H), 2.00-2.20(m, 2H), 2.70(t, J=6.9Hz, 2H) |
| | 4.19(t, J=6.1Hz, 2H), 7.04(d, J=9.0Hz, |
| | 2H), 7.96(d, J=9.0Hz, 2H). |
| 21-7 | 4-(4-Methoxybutoxy)benzenesulfonylchloride |
| | ¹H-NMR(250MHz, CDCl₃)δ; |
| | 1.70-2.00(m, 4H), 3.37(s, 3H), 3.47(t, J=6.1Hz, 2H), |
| | 4.12(t, J=6.3Hz, 2H), 7.04(d, J=9.1Hz, 2H), |
| | 7.98(d, J=9.1Hz, 2H). |
| 21-8 | 4-(3-Ethoxypropoxy)benzenesulfonylchloride |
| | ¹H-NMR(250MHz, CDCl₃)δ; |
| | 1.20(t, J=7.0Hz, 3H), 2.09(q, J=6.0Hz, 2H), 3.50(q, J= |
| | 7.0Hz, 2H), 3.59(t, J=6.0Hz, 2H), 4.18(t, |
| | J=6.0Hz, 2H), 7.05(d, J=9.1Hz, 2H), 7.97 |
| | (d, J=9.1Hz, 2H). |
| 21-9 | 4-Ethoxybenzenesulfonylchloride |
| | ¹H-NMR(250MHz, CDCl₃)δ; |
| | 1.47(t, J=7.0Hz, 3H), 4.15(q, J=7.0Hz, 2H), 7.02(d, J= |
| | 9.0Hz, 2H), 7.96(d, J=9.0Hz, 2H). |
| 21-10 | 4-Propoxybenzenesulfonylchloride |
| | ¹H-NMR(250MHz, CDCl₃)δ; |
| | 1.06(t, J=7.4Hz, 3H), 1.80-2.00(m, 2H), 4.03(t, J=6.5Hz, |
| | 2H), 7.03(d, J=9.1Hz, 2H), 7.97(d, J=9.1Hz, 2H). |
| 21-11 | 4-Trifluoromethoxybenzenesulfonylchloride |
| | ¹H-NMR(250MHz, CDCl₃)δ; |
| | 7.40-7.50(m, 2H), 8.10-8.20(m, 2H). |
| 21-12 | 4-(2-Fluoroethoxy)benzenesulfonylchloride |
| | ¹H-NMR(250MHz, CDCl₃)δ; |
| | 4.20-4.40(m, 2H), 4.70-4.90(m, 2H), 7.08(d, J=9.1Hz, |
| | 2H), 8.00(d, J=9.1Hz, 2H). |
| 21-13 | 4-Isobutoxybenzenesulfonylchloride |
| | ¹H-NMR(250MHz, CDCl₃)δ; |
| | 1.05(d, J=6.7Hz, 6H), 2.00-2.30(m, 1H), 3.83(d, J=6.5Hz, |
| | 2H), 7.03(d, J=9.0Hz, 2H), 7.97(d, J=9.0Hz, 2H). |
| 21-14 | 4-(3-Bromopropoxy)benzenesulfonylchloride |
| | ¹H-NMR(250MHz, CDCl₃)δ; |
| | 2.30-2.50(m, 2H), 3.63(t, J=6.3Hz, 2H), 4.25(t, J=5.8Hz, |
| | 2H), 7.00-7.20(m, 2H), 7.90-8.10(m, 2H). |
| 21-15 | 4-(3-Butoxypropoxy)benzenesulfonylchloride |
| | ¹H-NMR(250MHz, CDCl₃)δ; |
| | 0.91(t, J=7.2Hz, 3H), 1.20-1.70(m, 4H), 2.00-2.15 |
| | (m, 2H), 3.43(t, J=6.5Hz, 2H), 3.58(t, |
| | J=5.9Hz, 2H), 4.18(t, J=6.3Hz, 2H), 7.05(d, |
| | J=9.0Hz, 2H), 7.97(d, J=9.0Hz, 2H). |
| 21-16 | 4-(3-Isobutoxypropoxy)benzenesulfonylchloride |
| | ¹H-NMR(250MHz, CDCl₃)δ; |
| | 0.90(d, J=6.7Hz, 6H), 1.80-1.95(m, 1H), 2.00-2.20 |
| | (m, 2H), 3.20(d, J=6.7Hz, 2H), 3.58(t, |
| | J=6.0Hz, 2H), 4.19(t, J=6.3Hz, 2H), 7.05(d, |
| | J=9.1Hz, 2H), 7.97(d, J=9.1Hz, 2H). |
| 21-17 | 4-(2-Cyanoethoxy)benzenesulfonylchloride |
| | ¹H-NMR(250MHz, CDCl₃)δ; |
| | 2.94(t, J=6.2Hz, 2H), 4.32(t, J=6.2Hz, 2H), 7.09(d, J= |
| | 9.1Hz, 2H), 8.03(d, J=9.1Hz, 2H). |
| 21-18 | 4-Ethoxycarbonylbenzenesulfonylchloride |
| | ¹H-NMR(250MHz, CDCl₃)δ; |
| | 1.33(t, J=7.2Hz, 3H), 4.31(q, J=7.2Hz, 2H), 4.75(s, |
| | 2H), 7.06(d, J=9.0Hz, 2H), 8.00(d, J=9.0Hz, 2H). |
| 21-19 | 4-(3-Ethoxycarbonylpropoxy)benzenesulfonylchloride |
| | ¹H-NMR(250MHz, CDCl₃)δ; |
| | 1.28(t, J=7.1Hz, 3H), 2.10-2.30(m, 2H), 2.54(t, J=7.1Hz, |
| | 2H), 4.00-4.30(m, 4H), 7.05(d, J=9.0Hz, 2H), |
| | 7.99(d, J=9.0Hz, 2H). |
| 21-20 | 4-(3-Acetoxyethoxy)benzenesulfonylchloride |
| | ¹H-NMR(250MHz, CDCl₃)δ; |
| | 2.13(s, 3H), 4.20-4.40(m, 2H), 4.40-4.60(m, 2H), 7.08 |
| | (d, J=9.0Hz, 2H), 8.01(d, J=9.0Hz, 2H). |
| 21-21 | 4-(3-Methanesulfonylpropoxy)benzenesulfonylchloride |
| | ¹H-NMR(250MHz, CDCl₃)δ; |
| | 2.30-2.50(m, 2H), 2.99(s, 3H), 3.27(t, J=7.5Hz, 2H), |
| | 4.26(t, J=5.9Hz, 2H), 7.05(d, J=9.1Hz, 2H), |
| | 7.99(d, J=9.1Hz, 2H). |
| 21-22 | 5-Methylthiophen-2-sulfonylchloride |
| | ¹H-NMR(250MHz, CDCl₃)δ; |
| | 2.61(d, J=1.0Hz, 3H), 6.80-6.90(m, 1H), 7.71(d, J= |
| | 3.9Hz, 1H). |
| 21-23 | 5-(2-Methoxyethyl)thiophen-2-sulfonylchloride |
| | ¹H-NMR(250MHz, CDCl₃)δ; |
| | 3.16(dt, J=0.8 and 5.9Hz, 2H), 3.43(s, 3H), 3.65(t, J= |
| | 5.9Hz, 2H), 6.96(dt, J=0.8 and |
| | 3.9Hz, 1H), 7.16(d, J=3.9Hz, 1H). |

### Reference Example 22

Preparation of Sulfonylchloride(2)

A typical example is shown as follows by the preparation of 4-benzylbenzenesulfonyl chloride.

Diphenylmethane (12.5 g, 74.3 mmole) was dissolved in 1,2-dichloroethane (60 ml) and thereto was added chlorosulfonic acid (7.2 g, 59.44 mmole) and the mixture was stirred under nitrogen gas atmosphere at room temperature for 3 days. The solvent was distilled off under reduced pressure and thereto was added isooctane, and diphenylethane was azeotropically distilled. To the residue was added isooctane again and the precipitated solid was collected to give 4-benzylbenzenesulfonic acid (15.76 g) as pale green powders.

¹H-NMR(250 MHz, DMSO-d₆) δ; 3.92 (s, 2H), 6.27 (br s, 1H), 7.05-7.30 (m, 7H), 7.51 (d, J=6.9 Hz, 2H)

Next, 4-benzylbenzenesulfonic acid (2.0 g, 8.1 mmole) was dissolved in DMF (20 ml) and thereto was added thionyl chloride (2.0 ml, 26.9 mmole), and the mixture was stirred under ice-cooling for 15 minutes and at room temperature for an hour. After the reaction mixture was poured into ice water, it was extracted with a mixed solvent of diethylether/n-hexane (1/1). The organic layer was washed with ice-water and a saturated saline solution, then dried over magnesium sulfate. After magnesium sulfate was removed by filtration, the solvent was distilled off to give the title compound (1.87 g) as colorless oil.

In the same manner, 4-hexylbenzenesulfonylchloride was prepared. Physical constants of the prepared sulfonylchlorides were shown in Table 8.

TABLE 11

| No. | Physical constants of the obtained compounds |
|---|---|
| 22-1 | 4-Benzylbenzenesulfonylchloride |
| | ¹H-NMR(250MHz, CDCl₃)δ: 4.10(s, 2H), |
| | 7.10-7.50(m, 7H), 7.95(d, J=8.6Hz, 2H). |
| 22-2 | 4-Hexylbenzenesulfonylchloride |
| | ¹H-NMR(250MHz, CDCl₃)δ; 0.80-1.00(m, 3H), |
| | 1.20-1.40(m, 6H), 1.50-1.80(m, 2 |
| | H), 2.72(t, J=7.7Hz, 2H), 7.40(d, J=8.4Hz, 2H), |
| | 7.93(d, J=8.4Hz, 2H). |
| 22-3 | 4-(3-Phenylpropoxy)benzenesulfonylchloride |
| | ¹H-NMR(250MHz, CDCl₃)δ; 2.10-2.25(m, 2H), |
| | 2.83(t, J=7.5Hz, 2H), 4.06(t, J=6.3Hz, |
| | 2H), 7.01(d, J=9.1Hz, 2H), 7.15-7.35(m, 5H), |
| | 7.96(d, J=9.1Hz, 2H). |

### Reference Example 23

2-trans-(4-Methoxyphenyl)ethenesulfonyl Chloride

After 1-methoxy-4-vinylbenzene (5.5 g) was dissolved in chlorobenzene (6.5 ml), sulfur trioxide-pyridine complex (13.0 g) was added to the solution and the mixture was stirred at 110° C. for 18 hours. The solvent was distilled off to give 2-trans-(4-methoxyphenyl)ethensulfonic acid as yellow syrup.

The obtained 2-trans-(4-methoxyphenyl)ethensulfonic acid was mixed with phosphorus pentachloride (7.6 g) and the mixture was stirred at 85° C. for 10 minutes, at 50° C. for 3 hours and then at room temperature for 14 hours. The reaction mixture was poured into ice-water and the products were extracted with ethyl acetate. The ethyl acetate layer was washed with a saturated saline solution and dried over magnesium sulfate. After magnesium sulfate was removed, the solvent was distilled off and the residue was purified by a silica gel column chromatography of medium pressure (mobile phase; n-hexane/ethyl acetate=5/1) to give the title compound (1.15 g) as yellow solid.

¹H-NMR(250 MHz, CDCl₃) δ:3.88 (s, 3H), 6.97 (d, J=8.8 Hz, 2H), 7.10 (d, J=15.1 Hz, 1H), 7.45-7.60 (m, 2H), 7.69 (d, J=15.1 Hz, 1H).

### Reference Example 24

Preparation of 6-sulfonyl-5,6,7,8-tetrahydropyrido[3,4-b]pyrazine-7-carboxylic Acid Methyl Ester

A typical example is shown as follows by the preparation of methyl (+)-6-(4-methoxybenzenesulfonyl)-5,6,7,8-tetrahydropyrido[3,4-b]pyrazine-7-carboxylate

To methyl 5,6,7,8-tetrahydropyrido[3,4-b]pyrazine-7-carboxylate hydrochloride (0.64 g) was added DMF (10 ml) and thereto were added 4-dimethylaminopyridine (0.70 g) and 4-methoxybenzenesulfonyl chloride (0.60 g) and the mixture was stirred at room temperature overnight. The mixture was acidified with an 10% aquous citric acid and then extracted with ethyl acetate. The organic layer was washed with water and a saturated saline solution, and dried over magnesium sulfate. After removing magnesium sulfate, the solvent was distilled off, and the residue was purified by TLC (developing solution; ethyl acetate/n-hexane=1/1) to give the title compound (0.22 g) as colorless oil.

According to the example above, the following compounds were prepared.

TABLE 12

| No. | Physical constants of the obtained compounds |
|---|---|
| 24-1 | Methyl(±)-6-(4-methoxybenzenesulfonyl)- |
| | 5,6,7,8-tetrahydropyrido[3,4- |
| | b]pyrazine-7-carboxylate; |
| | ¹H-NMR(250MHz, CDCl₃)δ; 3.30-3.50(m, 2H), |
| | 3.47(s, 3H), 3.87(s, 3H), 4.54(d, |
| | J=16.9Hz, 1H), 4.90(d, J=16.9Hz, 1H), 5.19(dd, J=2.8 |
| | and 5.9Hz, 1H), 6.97(d, J=8.9Hz, |
| | 2H), 7.79(d, J=8.9Hz, 2H), 8.39(s, 2H). |
| 24-2 | Methyl(±)-6-[4-(pyridine-4-yloxy) |
| | benzenesulfonyl]-5,6,7,8-tetrahydro- |
| | pyrido[3,4-b]pyrazine-7-carboxylate; |
| | ¹H-NMR(250MHz, CDCl₃)δ; 3.40-3.50(m, 2H), |
| | 3.52(s, 3H), 4.58(d, J=16.7Hz, |
| | 1H), 4.94(d, J=16.7Hz, 1H), 5.23(dd, J=5.8 |
| | and 3.0Hz, 1H), 6.80-7.00(m, 2H), |
| | 7.21(d, J=9.0Hz, 2H), 7.93(d, J=9.0Hz, 2H), |
| | 8.42(s, 2H), 8.50-8.70(m, 2H). |
| 24-3 | Methyl(±)-6-(4-phenoxybenzenesulfonyl)- |
| | 5,6,7,8-tetrahydropyrido[3,4- |
| | b]pyrazine-7-carboxylate; |
| | ¹H-NMR(250MHz, CDCl₃)δ; 3.30-3.50(m, 2H), |
| | 3.50(s, 3H), 4.56(d, J=16.8Hz, |
| | 1H), 4.90(d, J=16.8Hz, 1H), 5.20(dd, J=5.7 |
| | and 3.1Hz, 1H), 7.00-7.10(m, 2H), |
| | 7.04(d, J=9.1Hz, 2H), 7.10-7.30(m, 1H), 7.30-7.50(m, 2H), |
| | 7.81(d, J=9.1Hz, 2H), |
| | 8.40(s, 2H). |

### Reference Example 25

Preparation of 6-sulfonyl-5,6,7,8-tetrahydropyrido[3,4-b]pyrazine-7-carboxylic Acid Derivative

A typical example is shown as follows by the preparation of (±)-6-(4-methoxybenzenesulfonyl)-5,6,7,8-tetrahydropyrido[3,4-b]pyrazine-7-carboxylic acid.

Methyl (±)-6-(4-methoxybenzenesulfonyl)-5,6,7,8-tetrahydropyrido[3,4-b]pyrazine-7-carboxylate (0.22 g) was dissolved in dioxane (3 ml) and thereto was added a 1N aqueous sodium hydroxide (1 ml) and the mixture was stirred at room temperature for 30 minutes. The mixture was acidified with an aqueous citric acid and then extracted with ethyl acetate. The organic layer was washed with water and a saturated saline solution, and dried over magnesium sulfate. After removing magnesium sulfate, the solvent was distilled off to give the title compound (0.14 g) as colorless powders.

According to the example above, the following compounds were prepared.

TABLE 13

| No. | Compounds |
|---|---|
| 25-1 | (±)-6-(4-Methoxybenzenesulfonyl)- |
| | 5,6,7,8-tetrahydropyrido[3,4-b]- |
| | pyrazine-7-carboxylic acid; |
| | ¹H-NMR(250MHz, CDCl₃)δ; 3.41(d, J=4.3Hz, 2H), 3.87(s, 3H), |
| | 4.57(d, J=17.0Hz, |
| | 1H), 4.88(d, J=17.0Hz, 1H), 5.22(t, J=4.3Hz, 1H), |
| | 6.96(d, J=8.9Hz, 2H), 7.81(d, |
| | J=8.9Hz, 2H), 8.38(s, 2H). |
| 25-2 | (±)-6-[4-(Pyridine-4-yloxy)benzenesulfonyl)- |
| | 5,6,7,8-tetrahydropyrido- |
| | [3,4-b]pyrazine-7-carboxylic acid hydrochloride; |
| | ¹H-NMR(250MHz, CDCl₃)δ; 2.95(dd, J=16.8 |
| | and 6.7Hz, 1H), 3.10-3.70(m, 1H), |
| | 4.40-4.70(m, 2H), 4.84(d, J=16.8Hz, 1H), |
| | 6.96(d, J=5.2Hz, 2H), 7.22(d, J=8.5Hz, |
| | 2H), 7.91(d, J=8.5Hz, 2H), 8.20-8.50(m, 2H), |
| | 8.51(d, J=5.2Hz, 2H). |
| 25-3 | (±)-6-(4-Phenoxybenzenesulfonyl)- |
| | 5,6,7,8-tetrahydropyrido[3,4-b]- |
| | pyrazine-7-carboxylic acid; |
| | ¹H-NMR(250MHz, CDCl₃)δ; 3.41(d, J=4.2Hz, 2H), |
| | 4.59(d, J=17.1Hz, 1H), 4.87(d, |
| | J=17.1Hz, 1H), 5.21(t, J=4.2Hz, 1H), 5.50-6.60(br, 1H), |
| | 6.90-7.10(m, 4H), 7.10-7.30 |
| | (m, 1H), 7.30-7.50(m, 2H), 7.80(d, J=9.0Hz, 2H), |
| | 8.36(d, J=2.5Hz, 1H), |
| | 8.39(d, J=2.5Hz, 1H). |

### Reference Example 26

Preparation of N-benzyloxy-6-sulfonyl-5,6,7,8-tetrahydropyrido[3,4-b]pyrazine-7-carboxylic Acid

A typical example is shown as follows by the preparation of (+)-N-benzyloxy-6-(4-methoxybenzenesulfonyl)-5,6,7,8-tetrahydropyrido[3,4-b]pyrazine-7-carboxamide;

(±)-6-(4-Methoxybenzenesulfonyl)-5,6,7,8-tetrahydropyrido[3,4-b]pyrazine-7-carboxylic acid (0.14 g) was dissolved in DMF (5 ml) and thereto were added WSC (0.12 g), HOBt (0.08 g), O-benzylhydroxylamine hydrochloride (0.10 g) and triethylamine (0.06 g), and the mixture was stirred at room temperature overnight. The reaction mixture was acidified with an aqueous citric acid and extracted with ethyl acetate. The organic layer was washed with a saturated aqueous sodium hydrogen carbonate solution, water and a saturated saline solution, and dried over magnesium sulfate. After removing magnesium sulfate, the solvent was distilled off to give the title compound (0.19 g) as pale yellow solids.

According to the example above, the following compounds were prepared.

TABLE 14

| No. | Physical constants of the obtained compounds |
|---|---|
| 26-1 | (±)-N-Benzyloxy-6-(4-methoxybenzenesulfonyl)- |
| | 5,6,7,8-tetrahydropyrido[3,4- |
| | b]pyrazine-7-carboxamide; |
| | ¹H-NMR(250MHz, CDCl₃)δ; δ; 2.86(dd, J=17.2 |
| | and 6.1Hz, 1H), 3.44(d, J=17.2Hz, |
| | 1H), 3.83(s, 3H), 4.40(d, J=18.0Hz, 1H), 4.70-5.00(m, |
| | 4H), 6.87(d, J=8.8Hz, 2H), |
| | 7.37(s, 5H), 7.66(d, J=8.8Hz, 2H), 8.33(s, 2H), 9.17(s, 1H). |
| 26-2 | (±)-N-Benzyloxy-6-[4-(pyridine-4- |
| | yloxy)benzenesulfonyl]-5,6,7,8-tetrahydro- |
| | pyrido[3,4-b]pyrazine-7-carboxamide; |
| | ¹H-NMR(250MHz, CDCl₃)δ; 2.90-3.10(m, 1H), |
| | 3.20-3.40(m, 1H), 4.50-4.70(m, 2 |
| | H), 4.60-4.90(m, 3H), 6.90-7.00(m, 2H), |
| | 7.20-7.40(m, 5H), 7.31(d, J=8.8Hz, 2H), |
| | 7.93(d, J=8.8Hz, 2H), 8.40-8.50(m, 2H), |
| | 8.45(s, 2H), 11.55(s, 1H). |
| 26-3 | (±)-N-Benzyloxy-6-(4-phenoxybenzenesulfonyl)- |
| | 5,6,7,8-tetrahydro- |
| | pyrido[3,4-b]pyrazine-7-carboxamide; |
| | ¹H-NMR(250MHz, CDCl₃)δ; 2.80-3.10(m, 1H), |
| | 3.30-3.60(m, 1H), 4.30-4.50(m, 1 |
| | H),4.60-5.00(m, 4H), 6.92(d, J=8.9Hz, 2H), |
| | 6.90-7.10(m, 2H), 7.10-7.30(m, 1H), |
| | 7.30-7.50(m, 7H), 7.66(d, J=8.9Hz, 2H), |
| | 8.35(s, 2H), 8.90-9.10(br, 1H). |

### Reference Example 27

Preparation of N-benzyloxy-6-sulfonyl-5,6,7,8-tetrahydropyrido[3,4-b]pyrazine-7-carboxylic Acid

A typical example is shown as follows by the preparation of (+)-N-benzyloxy-6-[4-(3-methoxypropoxy)benzenesulfonyl]-5,6,7,8-tetrahydropyrido[3,4-b]pyrazine-7-carboxamide.

(+)-N-Benzyloxy-5,6,7,8-tetrahydropyrido[3,4-b]pyrazine-7-carboxamide hydrochloride (0.65 g) was dissolved in a mixture of dioxane (15 ml) and water (15 ml) and thereto were added triethylamine (0.49 g) and 4-(3-methoxpropoxy)benzenesulfonyl (0.60 g) under ice-cooling, and the mixture was stirred at room temperature for 4 hours. To the reaction mixture was added water (50 ml), and the mixture was extracted with ethyl acetate. The organic layer was washed with a 1N hydrochloric acid, an aqueous sodium hydrogen carbonate solution, water and a saturated saline solution, and dried over magnesium sulfate. After removing magnesium sulfate, the solvent was distilled off, and the resulting residue was purified by a silica gel column chromatography (mobile phase; chloroform/methanol=30/1) to give the title compound (0.57 g) as pale brown powders.

According to the example above, the following compounds were prepared.

TABLE 15

| No. | Physical constants of the obtained compounds |
|---|---|
| 27-1 | (+)-N-Benzyloxy-6-[4- |
| | (3-methoxypropoxy)benzenesulfonyl]-5,6,7,8- |
| | tetrahydropyrido[3,4-b]pyrazine-7-carboxamide; |
| | ¹H-NMR(250MHz, CDCl₃)δ; 2.03(tt, J=6.2 |
| | and 6.1Hz, 2H), 2.70-3.00(m, 1H), 3.34 |
| | (s, 3H), 3.30-3.50(m, 1H), 3.52(t, J=6.1Hz, 2H), |
| | 4.06(t, J=6.2Hz, 2H), 4.38(d, |
| | J=18.3Hz, 1H), 4.70-5.00(m, 2H), 4.88(s, 2H), |
| | 6.87(d, J=8.9Hz, 2H), 7.37(s, 5H), |
| | 7.64(d, J=8.9Hz, 2H), 8.33(s, 2H), 9.05(s, 1H). |
| 27-2 | (±)-N-Benzyloxy-6-[4- |
| | (3-methoxypropoxy)benzenesulfonyl]-5,6,7,8- |
| | tetrahydropyrido[3,4-b]pyrazine-7-carboxamide; |
| | ¹H-NMR(250MHz, CDCl₃)δ; 2.03(tt, J=6.2 |
| | and 6.1Hz, 2H), 2.70-3.00(m, 1H), 3.34 |
| | (s, 3H), 3.30-3.50(m, 1H), 3.52(t, J=6.1Hz, 2H), |
| | 4.06(t, J=6.2Hz, 2H), 4.38(d, |
| | J=18.3Hz, 1H), 4.70-5.00(m, 2H), 4.88(s, 2H), |
| | 6.87(d, J=8.9Hz, 2H), 7.37(s, 5H), |
| | 7.64(d, J=8.9Hz, 2H), 8.33(s, 2H), 9.05(s, 1H). |
| 27-3 | (±)-N-Benzyloxy-6-[4- |
| | (pyrazine-2-yloxy)benzenesulfonyl]-5,6,7,8-tetra- |
| | hydropyrido[3,4-b]pyrazine-7-carboxamide; |
| | ¹H-NMR(250MHz, CDCl₃)δ; 2.95(dd, J=17.2 |
| | and 6.0Hz, 1H), 3.45(d, J=17.2Hz, |
| | 1H), 4.45(d, J=18.0Hz, 1H), 4.70-5.00(m, 4H), |
| | 7.23(d, J=8.7Hz, 2H), 7.36(s, 5 |
| | H), 7.79(d, J=8.7Hz, 2H), 8.00-8.10(m, 1H), |
| | 8.30-8.40(m, 3H), 8.40-8.50(m, 1H), |
| | 9.10(s, 1H). |
| 27-4 | (±)-N-Benzyloxy-6- |
| | [4-(4-nitrophenoxy)benzenesulfonyl]-5,6,7,8-tetra- |
| | hydropyrido[3,4-b]pyrazine-7-carboxamide; |
| | ¹H-NMR(250MHz, CDCl₃)δ; 2.90-3.10(m, 1H), |
| | 3.44(d, J=17.7Hz, 1H), 4.48(d, |
| | J=16.7Hz, 1H), 4.80-5.00(m, 4H), 7.00-7.10(m, 4H), |
| | 7.38(s, 5H), 7.79(d, J=8.7Hz, |
| | 2H), 8.20-8.30(m, 2H), 8.37(s, 1H), 9.08(s, 1H). |
| 27-5 | (±)-N-Benzyloxy-6-[4- |
| | (2-ethoxyethoxy)benzenesulfonyl]-5,6,7,8-tetra- |
| | hydropyrido[3,4-b]pyrazine-7-carboxamide; |
| | ¹H-NMR(250MHz, CDCl₃)δ; |
| | 1.24(t, J=7.0Hz, 3H), 2.70-3.00(m, 1H), 3.40-3.60 |
| | (m, 1H), 3.59(q, J=7.0Hz, 2H), 3.70-3.80(m, 2H), |
| | 4.10-4.20(m, 2H), 4.39(d, J=17.8Hz, |
| | 1H), 4.70-5.00(m, 4H), 6.91(d, J=8.9Hz, 2H), |
| | 7.38(s, 5H), 7.65(d, J=8.9Hz, |
| | 2H), 8.34(s, 2H), 9.09(s, 1H). |
| 27-6 | (±)-N-Benzyloxy-6-[4- |
| | (2-methoxyethoxy)benzenesulfonyl]-5,6,7,8- |
| | tetrahydropyrido[3,4-b]pyrazine-7-carboxamide; |
| | ¹H-NMR(250MHz, CDCl₃)δ; 2.70-2.90(m, 1H), |
| | 3.43(s, 3H), 3.40-3.50(m, 1H), |
| | 3.70-3.80(m, 2H), 4.00-4.20(m, 2H), |
| | 4.38(d, J=17.0Hz, 1H), 4.70-5.00(m, 4H), 6.89 |
| | (d, J=8.9Hz, 2H), 7.37(s, 5H), 7.64(d, J=8.9Hz, |
| | 2H), 8.32(s, 2H), 9.06(s, 1H). |
| 27-7 | (±)-N-Benzyloxy-6- |
| | (4-pentyloxybenzenesulfonyl)-5,6,7,8-tetrahydro- |
| | pyrido[3,4-b]pyrazine-7-carboxamide; |
| | ¹H-NMR(250MHz, CDCl₃)δ; |
| | 0.94(t, J=7.0Hz, 3H), 1.30-1.60(m, 4H), 1.70-1.90 |
| | (m, 2H), 2.80-3.00(m, 1H), 3.46(d, J=17.4Hz, 1H), |
| | 3.96(t, J=6.5Hz, 2H), 4.40(d, |
| | J=16.9Hz, 1H), 4.70-5.10(m, 4H), |
| | 6.86(d, J=8.9Hz, 2H), 7.37(s, 5H), 7.65(d, J=8.9Hz, |
| | 2H), 8.34(s, 2H), 9.14(br s, 1H). |
| 27-8 | (±)-N-Benzyloxy- |
| | 6-[4-(4-methoxybutoxy)benzenesulfonyl]-5,6,7,8- |
| | tetrahydropyrido[3,4-b]pyrazine-7-carboxamide; |
| | ¹H-NMR(250MHz, CDCl₃)δ; |
| | 1.60-2.00(m, 4H), 2.70-3.00(m, 1H), 3.34(s, 3H), |
| | 3.30-3.60(m, 3H), 3.99(t, J=6.2Hz, 2H), |
| | 4.38(d, J=16.9Hz, 1H), 4.70-5.00(m, 4 |
| | H), 6.85(d, J=8.9Hz, 2H), 7.37(s, 5H), |
| | 7.63(d, J=8.9Hz, 2H), 8.33(br s, 2H), 9.10 |
| | (brs, 1H). |
| 27-9 | (±)-N-Benzyloxy-6-[4- |
| | (3-ethoxypropoxy)benzenesulfonyl]-5,6,7,8-tetra- |
| | hydropyrido[3,4-b]pyrazine-7-carboxamide; |
| | ¹H-NMR(250MHz, DMSO-d₆)δ; |
| | 1.06(t, J=7.0Hz, 3H), 1.89(q, J=6.2Hz, 2H), 2.94 |
| | (d, J=7.0Hz, 1H), 3.10-3.20(m, 1H), |
| | 3.30-3.50(m, 4H), 4.00-4.10(m, 2H), 4.50-4.70 |
| | (m, 4H), 4.74(d, J=5.8Hz, 1H), |
| | 7.06(d, J=8.8Hz, 2H), 7.20-7.30(m, 5H), 7.75 |
| | (d, J=8.8Hz, 2H), 8.42(d, J=3.0Hz, 2H), 11.53(s, 1H). |
| 27-10 | (+)-N-Benzyloxy-6- |
| | (4-methoxybenzenesulfonyl)-5,6,7,8-tetrahydropyrido[3,4-b] |
| | pyrazine-7-carboxamide; |
| | ¹H-NMR(250MHz, CDCl₃)δ; |
| | 2.86(dd, J=17.2 and 6.1Hz, 1H), 3.44(d, J=17.2Hz, |
| | 1H), 3.83(s, 3H), 4.40(d, J=18.0Hz, 1H), |
| | 4.70-5.00(m, 4H), 6.87(d, J=8.8Hz, 2H), |
| | 7.37(s, 5H), 7.66(d, J=8.8Hz, 2H), 8.33(s, 2H), 9.17(s, 1H). |
| | [α]_{D}; +41° (c=1.0, methanol) |
| 27-11 | (+)-N-Benzyloxy-6- |
| | (4-ethoxybenzenesulfonyl)-5,6,7,8-tetrahydropyrido[3,4-b]- |
| | pyrazine-7-carboxamide; |
| | ¹H-NMR(250MHz, DMSO-d₆)δ; |
| | 1.31(t, J=7.0Hz, 3H), 2.96(d, J=15.8Hz, 1H), 3.10-3.30 |
| | (m, 1H), 4.08(q, J=7.0Hz, 2H), 4.50-4.80(m, |
| | 5H), 7.07(d, J=8.9Hz, 2H), |
| | 7.20-7.40(m, 5H), 7.77(d, J=8.9Hz, 2H), |
| | 8.32(s, 1H), 8.43(d, J=2.6Hz, 1H), 8.45 |
| | (d, J=2.6Hz, 1H). |
| 27-12 | (+)-N-Benzyloxy-6- |
| | (4-propoxybenzenesulfonyl)-5,6,7,8-tetrahydropyrido[3,4-b] |
| | pyrazine-7-carboxamide; |
| | ¹H-NMR(250MHz, DMSO-d₆)δ; |
| | 0.90(t, J=7.4Hz, 3H), 1.60-1.80(m, 2H), 2.93(d, |
| | J=16.1Hz, 1H), 3.10-3.30(m, 1H), |
| | 3.90-4.00(m, 2H), 4.50-4.80(m, 5H), 7.05(d, |
| | J=8.9Hz, 2H), 7.20-7.40(m, 5H), |
| | 7.75(d, J=8.9Hz, 2H), 8.29(s, 1H), 8.40(d, J=2.5Hz, |
| | 1H), 8.43(d, J=2.5Hz, 1H). |
| 27-13 | (+)-N-Benzyloxy-6- |
| | (4-trifluoromethoxybenzenesulfonyl)-5,6,7,8-tetrahydropyrido |
| | [3,4-b]pyrazine-7-carboxamide; |
| | ¹H-NMR(250MHz, CDCl₃)δ; |
| | 2.80-3.00(m, 1H), 3.20-3.40(m, 1H), 4.40-4.50(m, |
| | 1H), 4.70-5.00(m, 4H), 7.24(d, J=8.9Hz, |
| | 2H), 7.38(s, 5H), 7.78(d, J=8.9Hz, 2H), |
| | 8.33(s, 2H), 9.00(s, 1H). |
| 27-14 | (+)-N-Benzyloxy-6-[4- |
| | (2-fluoroethoxy)benzenesulfonyl]-5,6,7,8-tetrahydropyrido |
| | [3,4-b]pyrazine-7-carboxamide; |
| | ¹H-NMR(250MHz, CDCl₃)δ; |
| | 2.86(dd, J=5.6 and 17.5Hz, 1H), 3.45(d, J=17.5Hz, |
| | 1H), 4.10-4.30(m, 2H), 4.38(d, J=17.8Hz, 1H), |
| | 4.60-4.90(m, 6H), 6.90(d, J=8.9Hz, |
| | 2H), 7.37(s, 5H), 7.67(d, J=8.9Hz, 2H), 8.33(s, 2H), 9.00(s, 1H). |
| 27-15 | (+)-N-Benzyloxy-6- |
| | [4-(2-methoxyethoxy)benzenesulfonyl]-5,6,7,8- |
| | tetrahydropyrido[3,4-b]pyrazine-7-carboxamide; |
| | ¹H-NMR(250MHz, CDCl₃)δ; |
| | 2.70-2.90(m, 1H), 3.43(s, 3H), 3.40-3.50(m, 1H), |
| | 3.70-3.80(m, 2H), 4.00-4.20(m, 2H), |
| | 4.38(d, J=17.0Hz, 1H), 4.70-5.00(m, 4H), 6.89 |
| | (d, J=8.9Hz, 2H), 7.37(s, 5H), 7.64(d, |
| | J=8.9Hz, 2H), 8.32(s, 2H), 9.06(s, 1H). |
| 27-16 | (+)-N-Benzyloxy-6- |
| | (4-phenoxybenzenesulfonyl)-5,6,7,8-tetrahydropyrido[3,4-b] |
| | pyrazine-7-carboxamide; |
| | ¹H-NMR(250MHz, CDCl₃)δ; |
| | 2.87(dd, J=7.1 and 17.6Hz, 1H), 3.45(d, J=17.6Hz, |
| | 1H), 4.40(d, J=17.8Hz, 1H), 4.60-5.00(m, 4H), |
| | 6.92(d, J=8.9Hz, 2H), 6.90-7.10 |
| | (m, 2H), 7.10-7.30(m, 1H), 7.30-7.50(m, 7H), |
| | 7.66(d, J=8.9Hz, 2H), 8.35(s, 2H), |
| | 8.90-9.10(br s, 1H). |
| 27-17 | (+)-N-Benzyloxy-6- |
| | (4-isobutoxybenzenesulfonyl)-5,6,7,8-tetrahydropyrido[3,4- |
| | b]pyrazine-7-carboxamide; |
| | ¹H-NMR(250MHz, CDCl₃)δ; |
| | 1.01(d, J=6.7Hz, 6H), 1.90-2.20(m, 1H), 2.84(dd, J= |
| | 6.8 and 17.0Hz, 1H), 3.46(d, J=17.0Hz, 1H), |
| | 3.71(d, J=6.5Hz, 2H), 4.37(d, J=17.8Hz, |
| | 1H), 4.70-5.00(m, 4H), 6.85(d, J=8.9Hz, 2H), |
| | 7.30-7.50(m, 5H), 7.63(d, J= |
| | 8.9Hz, 2H), 8.34(s, 2H), 9.09(br s, 1H). |
| 27-18 | (+)-N-Benzyloxy-6-[4- |
| | (3-butoxypropoxy)benzenesulfonyl]-5,6,7,8- |
| | tetrahydropyrido[3,4-b]pyrazine-7-carboxamide; |
| | ¹H-NMR(250MHz, CDCl₃)δ; |
| | 0.90(t, J=7.3Hz, 3H), 1.20-1.70(m, 4H), 1.90-2.10 |
| | (m, 2H), 2.70-3.00(m, 1H), 3.30-3.50(m, 3H), |
| | 3.55(t, J=6.0Hz, 2H), 4.07(t, J=6.3Hz, |
| | 2H), 4.38(d, J=17.1Hz, 1H), 4.70-5.00(m, 4H), |
| | 6.87(d, J=8.9Hz, 2H), 7.30-7.50 |
| | (m, 5H), 7.64(d, J=8.9Hz, 2H), 8.34(s, 2H), 9.10(br s, 1H). |
| 27-19 | (+)-N-Benzyloxy-6-[4- |
| | (3-isobutoxypropoxy)benzenesulfonyl]-5,6,7,8- |
| | tetrahydropyrido[3,4-b]pyrazine-7-carboxamide; |
| | ¹H-NMR(250MHz, CDCl₃)δ; |
| | 0.88(d, J=7.1Hz, 6H), 1.70-2.10(m, 3H), 2.70-2.90 |
| | (m, 1H), 3.18(d, J=6.7Hz, 2H), 3.46(d, J=18.0Hz, |
| | 1H), 3.55(t, J=6.0Hz, 2H), 4.08 |
| | (t, J=6.3Hz, 2H), 4.38(d, J=18.4Hz, |
| | 1H), 4.70-5.00(m, 4H), 6.87(d, J=8.8Hz, 2H), |
| | 7.30-7.50(m, 5H), 7.64(d, J=8.8Hz, 2H), |
| | 8.30-8.40(m, 2H), 9.13(br s, 1H). |
| 27-20 | (+)-N-Benzyloxy-6-[4- |
| | (4-nitrophenoxy)benzenesulfonyl-5,6,7,8-tetrahydropyrido |
| | [3,4-b]pyrazine-7-carboxamide; |
| | ¹H-NMR(250MHz, CDCl₃)δ; |
| | 2.98(dd, J=7.4 and 17.3Hz, 1H), 3.39(d, J=17.3Hz, |
| | 1H), 4.50(d, J=17.6Hz, 1H), 4.70-5.00(m, |
| | 4H), 6.90-7.20(m, 4H), 7.20-7.50(m, 5H), |
| | 7.78(d, J=8.7Hz, 2H), 8.23(d, |
| | J=9.1Hz, 2H), 8.35(s, 2H), 9.28(br s, 1H). |
| 27-21 | (+)-N-Benzyloxy-6- |
| | (4-pivaloyloxybenzenesulfonyl)-5,6,7,8-tetrahydropyrido |
| | [3,4-b]pyrazine-7-carboxamide; |
| | ¹H-NMR(250MHz, CDCl₃)δ; |
| | 1.35(s, 9H), 2.91(dd, J=6.6 and 17.8Hz, 1H), 3.44 |
| | (d, J=17.8Hz, 1H), 4.43(d, J=17.2Hz, 1H), |
| | 4.70-5.00(m, 4H), 7.15(d, J=8.5Hz, 2H), |
| | 7.30-7.50(m, 5H), 7.76(d, J=8.5Hz, 2H), |
| | 8.35(s, 2H), 9.15(s, 1H). |
| 27-22 | (+)-N-Benzyloxy-6-[4- |
| | (2-cyanoethoxy)benzenesulfonyl]-5,6,7,8-tetrahydropyrido |
| | [3,4-b]pyrazine-7-carboxamide; |
| | ¹H-NMR(250MHz, CDCl₃)δ; |
| | 2.77(t, J=6.1Hz, 2H), 2.91(dd, J=7.0 and 17.3Hz, |
| | 1H), 3.28(d, J=17.3Hz, 1H), 4.11(t, J=6.1Hz, 2H), |
| | 4.48(d, J=17.6Hz, 1H), 4.60-4.90 |
| | (m, 4H), 6.84(d, J=8.8Hz, 2H), 7.10-7.50(m, 5H), |
| | 7.66(d, J=8.8Hz, 2H), 8.28 |
| | (s, 2H), 9.79(s, 1H). |
| 27-23 | (+)-N-Benzyloxy-6- |
| | (4-ethoxycarbonylmethoxybenzenesulfonyl)- |
| | 5,6,7,8-tetrahydropyrido |
| | [3,4-b]pyrazine-7-carboxamide; |
| | ¹H-NMR(250MHz, CDCl₃)δ; |
| | 1.31(t, J=7.1Hz, 3H), 2.88(dd, J=7.7 and 16.9Hz, 1H), |
| | 3.45(d, J=16.9Hz, 1H), 4.28(q, J=7.1Hz, 2H), |
| | 4.40(d, J=17.2Hz, 1H), 4.64(s, |
| | 2H), 4.70-5.00(m, 4H), 6.89(d, J=8.9Hz, 2H), |
| | 7.30-7.50(m, 5H), 7.68(d, J=8.9Hz, |
| | 2H), 8.30-8.40(m, 2H), 9.17(s, 1H). |
| 27-24 | (+)-N-Benzyloxy-6-(4-methylbenzenesulfonyl)- |
| | 5,6,7,8-tetrahydropyrido[3,4-b]- |
| | pyrazine-7-carboxamide; |
| | ¹H-NMR(250MHz, CDCl₃)δ; 2.37(s, 3H), |
| | 2.82(dd, J=6.8 and 17.2Hz, 1H), 3.44 |
| | (d, J=17.2Hz, 1H), 4.39(d, J=18.2Hz, 1H), |
| | 4.70-5.00(m, 4H), 7.21(d, J=8.1Hz, 2H), |
| | 7.30-7.50(m, 5H), 7.61(d, J=8.1Hz, 2H), |
| | 8.33(s, 2H), 9.09(br s, 1H). |
| 27-25 | (+)-N-Benzyloxy-6-[4-(3- |
| | ethoxycarbonylpropoxy)benzenesulfonyl]-5,6,7,8-tetrahydropyrido |
| | [3,4-b]pyrazine-7-carboxamide; |
| | ¹H-NMR(250MHz, CDCl₃)δ; |
| | 1.27(t, J=7.1Hz, 3H), 2.00-2.20(m,2H), 2.51(t, J= |
| | 7.2Hz, 2H), 2.85(dd, J=17.5 and 7.3Hz, 1H), |
| | 3.46(d, J=17.5Hz, 1H), 4.03(t, J=6.1Hz, |
| | 2H), 4.16(q, J=7.1Hz, 2H), 4.39(d, J=17.2Hz, |
| | 1H), 4.70-4.90(m, 4H), 6.87(d, |
| | J=8.8Hz, 2H), 7.20-7.50(m, 5H), |
| | 7.65(d, J=8.8Hz, 2H), 8.35(s, 2H), 9.13(br s, 1H). |
| 27-26 | (+)-N-Benzyloxy-6-[4-(2- |
| | acetoxyethoxy)benzenesulfonyl]-5,6,7,8- |
| | tetrahydropyrido[3,4-b]pyrazine-7-carboxamide; |
| | ¹H-NMR(250MHz, CDCl₃)δ; |
| | 2.12(s, 3H), 2.85(dd, J=17.3 and 7.1Hz, 1H), 3.46 |
| | (d, J=17.3Hz, 1H), 4.10-4.20(m, 2H), |
| | 4.30-4.50(m, 3H), 4.70-5.00(m, 4H), 6.90 |
| | (d, J=8.8Hz, 2H), 7.20-7.50(m, 5H), |
| | 7.68(d, J=8.8Hz, 2H), 8.36(s, 2H), 9.09(br s, |
| | 1H). |
| 27-27 | (+)-N-Benzyloxy-6-(3- |
| | methylybenzenesulfonyl)-5,6,7,8-tetrahydropyrido[3,4-b] |
| | pyrazine-7-carboxamide; |
| | ¹H-NMR(250MHz, CDCl₃)δ; |
| | 2.34(s, 3H), 2.83(dd, J=7.2 and 17.5Hz, 1H), 3.43 |
| | (d, J=17.5Hz, 1H), 4.41(d, J=17.9Hz, 1H), |
| | 4.70-5.00(m, 4H), 7.20-7.60(m, 9H), |
| | 8.32(s, 2H), 9.16(br s, 1H). |
| 27-28 | (+)-N-Benzyloxy-6-benzenesulfonyl- |
| | 5,6,7,8-tetrahydropyrido[3,4-b]pyrazine- |
| | 7-carboxamide; |
| | ¹H-NMR(250MHz, CDCl₃)δ; |
| | 2.82(dd, J=7.4 and 17.5Hz, 1H), 3.42(d, J=17.5Hz, |
| | 1H), 4.41(d, J=18.1Hz, 1H), 4.70-5.00(m, 4H), |
| | 7.30-7.60(m, 8H), 7.72(d, J=7.4Hz, |
| | 2H), 8.32(s, 2H), 9.13(s, 1H). |
| 27-29 | (+)-N-Benzyloxy-6- |
| | (4-fluorobenzenesulfonyl)-5,6,7,8-tetrahydropyrido[3,4-b]- |
| | pyrazine-7-carboxamide; |
| | ¹H-NMR(250MHz, CDCl₃)δ; |
| | 2.80-3.00(m, 1H), 3.42(d, J=18.4Hz, 1H), 4.41(d, J= |
| | 17.7Hz, 1H), 4.70-5.00(m, 4H), 7.00-7.20(m, 2H), |
| | 7.20-7.50(m, 5H), 7.70-7.80 |
| | (m, 2H), 8.30-8.40(m, 2H), 9.05(s, 1H). |
| 27-30 | (+)-N-Benzyloxy-6-(4- |
| | hexylbenzenesulfonyl)-5,6,7,8-tetrahydropyrido-[3,4-b]- |
| | pyrazine-7-carboxamide; |
| | ¹H-NMR(250MHz, CDCl₃)δ; |
| | 0.80-1.00(m, 3H), 1.20-1.40(m, 6H), 1.50-1.70(m, |
| | 2H), 2.59(t, J=7.7Hz, 2H), 2.82(dd, J=8.5 |
| | and 17.7Hz, 1H), 3.44(d, J=17.7Hz, 1H), |
| | 4.38(d, J=17.7Hz, 1H), 4.70-5.00(m, 4H), |
| | 7.19(d, J=8.2Hz, 2H), 7.20-7.50(m, |
| | 5H), 7.60(d, J=8.2Hz, 2H), 8.31(s, 2H), 9.11(br s, 1H). |
| 27-31 | (+)-N-Benzyloxy-6-(4- |
| | nitrobenzenesulfonyl)-5,6,7,8-tetrahydropyrido[3,4-b]- |
| | pyrazine-7-carboxamide; |
| | ¹H-NMR(250MHz, CDCl₃)δ; |
| | 2.90-3.10(m, 1H), 3.30-3.50(m, 1H), 4.40-4.70(m, |
| | 1H), 4.80-5.10(m, 4H), 7.20-7.50(m, 5H), |
| | 7.94(d, J=8.9Hz, 2H), 8.29(d, J=8.9Hz, |
| | 2H), 8.37(s, 2H), 8.91(br s, 1H). |
| 27-32 | (+)-N-Benzyloxy-6-(biphenyl-4-sulfonyl)- |
| | 5,6,7,8-tetrahydropyrido[3,4-b]- |
| | pyrazine-7-carboxamide; |
| | ¹H-NMR(250MHz, CDCl₃)δ; |
| | 2.80-3.00(m, 1H), 3.47(d, J=16.8Hz, 1H), 4.44(d, J= |
| | 18.0Hz, 1H), 4.80-5.00(m, 4H), 7.30-7.70(m, 12H), |
| | 7.78(d, J=8.2Hz, 2H), 8.30-8.40 |
| | (m, 2H), 9.12(br s, 1H). |
| 27-33 | (+)-N-Benzyloxy-6-(4-propylbenzenesulfonyl)- |
| | 5,6,7,8-tetrahydropyrido[3,4-b]- |
| | pyrazine-7-carboxamide; |
| | ¹H-NMR(250MHz, CDCl₃)δ; |
| | 0.90(d, J=7.3Hz, 3H), 1.50-1.70(m, 2H), 2.59(t, J= |
| | 7.6Hz, 2H), 2.83(dd, J=7.8 and 17.5Hz, 1H), |
| | 3.45(d, J=17.5Hz, 1H), 4.38(d, J=18.2Hz, |
| | 1H), 4.70-5.00(m, 4H), 7.20(d, J=8.2Hz, 2H), |
| | 7.30-7.50(m, 5H), 7.62(d, J= |
| | 8.2Hz, 2H), 8.32(s, 2H), 9.06(br s, 1H). |

### Reference Example 28

(±)-6-[4-(3-Methylthiopropoxy)benzenesulfonyl]-5,6,7,8-tetrahydropyrido[3,4-b]-pyrazine-7-carboxylic Acid

(a) tert-Butyl (±)-6-[4-(3-methylthiopropoxy)benzenesulfonyl]-5,6,7,8-tetrahydropyrido-[3,4-b]pyrazine-7-carboxylate;

To tert-butyl (±)-5,6,7,8-tetrahydropyrido[3,4-b]pyrazine-7-carboxylate obtained (0.87 g) were added dioxane (5 ml), water (5 ml) and dichloromethane (3 ml) and thereto were further added triethylamine (0.41 g), 4-(3-methylthiopropoxy)benzenesulfonyl chloride (1.14 g) and the mixture was stirred at room temperature overnight. The reaction mixture was acidified with an aqueous citric acid solution and was extracted with ethyl acetate. The organic layer was washed with water and a saturated saline solution, and dried over magnesium sulfate. After removing magnesium sulfate, the residue was purified by a silica gel column chromatography (mobile phase; ethyl acetate/n-hexane=1/1) to give the title compound (1.4 g) as pale yellow solids.

¹H-NMR (250 MHz, CDCl₃) δ; 1.19 (9H, s), 2.0-2.2 (5H, m), 2.6-2.8 (2H, m), 3.3-3.5 (2H, m), 4.0-4.2 (2H, m), 4.60 (1H, d, J=16.8 Hz), 4.90 (1H, d, J=16.8 Hz), 5.0-5.1 (1H, m), 6.98 (2H, d, J=8.8 Hz), 7.81 (2H, d, J=8.8 Hz), 8.40 (2H, s).

(b) (±)-6-[4-(3-Methylthiopropoxy)benzenesulfonyl]-5,6,7,8-tetrahydropyrido[3,4-b]pyrazine-7-carboxylic acid;

To tert-butyl (±)-6-[4-(3-methylthiopropoxy)benzenesulfonyl]-5,6,7,8-tetrahydropyrido-[3,4-b]pyrazine-7-carboxylate (1.4 g) was added 90% aqueous trifluoroacetic acid (10 ml) under ice-cooling, and the mixture was stirred for 3.5 hours. After distilling off the solvent, the mixture was extracted with ethyl acetate. The organic layer was washed with water and a saturated saline solution, and dried over magnesium sulfate. After removing magnesium sulfate, the solvent was distilled off, and the residue was purified by a silica gel column chromatography (mobile phase; chloroform/methanol=5/1) to give the title compound (0.66 g).

¹H-NMR (250 MHz, CDCl₃) δ; 2.13 (3H, s), 2.0-2.2 (2H, m), 2.69 (2H, t, J=7.0 Hz), 3.4-3.5 (2H, m), 4.14 (2H, t, J=6.1 Hz), 4.61 (1H, d, J=17.4 Hz), 4.93 (1H, d, J=17.4 Hz), 5.2-5.3 (1H, m), 6.98 (2H, d, J=8.8 Hz), 7.80 (2H, d, J=8.8 Hz), 8.4-8.6 (2H, m).

### Reference Example 29

Preparation of (+)-6-sulfonyl-5,6,7,8-tetrahydropyrido[3,4-b]pyrazine-7-carboxylic Acid

A typical example is shown as follows by the preparation of (+)-6-[4-(3-methylthiopropoxy)benzenesulfonyl]-5,6,7,8-tetrahydropyrido[3,4-b]pyrazine-7-carboxylic acid.

(+)-5,6,7,8-tetrahydropyrido[3,4-b]pyrazine-7-carboxylic acid hydrochloride (1.20 g) was dissolved in a mixture of dioxane (10 ml) and water (10 ml) and thereto were added triethylamine (1.40 g) and 4-(3-methylthiopropoxy)benzenesulfonyl chloride (1.80 g) and the mixture was stirred at room temperature for 4 hours. The reaction mixture was acidified with a diluted hydrochloric acid and extracted with ethyl acetate. The organic layer was washed with water and a saturated saline solutions, and dried over magnesium sulfate. After removing magnesium sulfate, the residue was purified by a silica gel column chromatography (mobile phase; chloroform/methanol=10/1) to give the title compound (0.79 g).

According to the example above, the following compounds were prepared.

TABLE 16

| No. | Physical constants of the obtained compounds |
|---|---|
| 29-1 | (+)-6-[4-(3-Methylthiopropoxy)benzenesulfonyl]- |
| | 5,6,7,8-tetrahydropyrido[3,4-b]- |
| | pyrazine-7-carboxylic acid |
| | ¹H-NMR(250MHz, CDCl₃)δ; |
| | 2.13(s, 3H), 2.00-2.20(m, 2H), 2.69(t, J=7.0Hz, 2H), |
| | 3.40-3.50(m, 2H), 4.14(t, J=6.1Hz, 2H), |
| | 4.61(d, J=17.4Hz, 1H), 4.93(d, J=17.4Hz, |
| | 1H), 5.20-5.30(m, 1H), 6.98(d, J=8.8Hz, 2H), |
| | 7.80(d, J=8.8Hz, 2H), 8.30-8.50(m, |
| | 2H). |
| 29-2 | (+)-6-[4-(3-Ethoxypropoxy)benzenesulfonyl]- |
| | 5,6,7,8-tetrahydropyrido[3,4-b]- |
| | pyrazine-7-carboxylic acid |
| | ¹H-NMR(250MHz, CDCl₃)δ; |
| | 1.18(t, J=7.0Hz, 3H), 1.90-2.20(m, 2H), 2.60-2.80 |
| | (m, 2H), 3.49(q, J=7.0Hz, 2H), |
| | 3.59(t, J=6.1Hz, 2H), 4.08(t, J=5.9Hz, 2H), 4.58(d, |
| | J=16.9Hz, 1H), 4.89(d, J=16.9Hz, 1H), |
| | 5.19(dd, J=3.2 and 5.6Hz, 1H), 6.95(d, J=8.9Hz, |
| | 2H), 7.79(d, J=8.9Hz, 2H), 8.35(d, |
| | J=2.7Hz, 1H), 8.38(d, J=2.7Hz, 1H). |
| 29-3 | (+)-6-[4-(3-Bromopropoxy)benzenesulfonyl]- |
| | 5,6,7,8-tetrahydropyrido[3,4-b]- |
| | pyrazine-7-carboxylic acid |
| | ¹H-NMR(250MHz, CDCl₃)δ; |
| | 2.20-2.50(m, 2H), 3.42(d, J=4.2Hz, 2H), 3.61(t, J=6.3Hz, |
| | 2H), 4.17(t, J=6.0Hz, 2H), 4.63(d, J=17.1Hz, 1H), |
| | 4.89(d, J=17.1Hz, 1H), 5.23 |
| | (t, J=4.2Hz, 1H), 6.90-7.10(m, 2H), 7.70-7.90(m, |
| | 2H), 8.34(d, J=2.5Hz, 1H), 8.42 |
| | (d, J=2.5Hz, 1H). |
| 29-4 | (+)-6-[4-(2-Ethoxyethoxy)benzenesulfonyl]- |
| | 5,6,7,8-tetrahydropyrido[3,4-b]- |
| | pyrazine-7-carboxylic acid |
| | ¹H-NMR(250MHz, DMSO-d₆)δ; |
| | 1.09(t, J=7.0Hz, 3H), 3.14(dd, J=2.3 and 17.4Hz, |
| | 1H), 3.25(dd, J=6.4 and J=17.4Hz, 1H), |
| | 3.46(q, J=7.0Hz, 2H), 3.60-3.70(m, 2H), |
| | 4.00-4.20(m, 2H), 4.46(d, J=17.2Hz, 1H), |
| | 4.68(d, 17.2Hz, 1H), 5.02(dd, J=6.4 and |
| | 2.3Hz, 1H), 7.06(d, J=8.9Hz, 2H), |
| | 7.76(d, J=8.9Hz, 2H), 8.40-8.50(m, 2H), 13.15 |
| | (br s, 1H). |
| 29-5 | (+)-6-[4-(Pyrazine-2-yloxy)benzenesulfonyl]- |
| | 5,6,7,8-tetrahydropyrido[3,4-b]- |
| | pyrazine-7-carboxylic acid |
| | ¹H-NMR(250MHz, CDCl₃)δ; |
| | 3.40-3.70(m, 2H), 4.68(d, J=16.7Hz, 1H), 5.00(d, J= |
| | 16.7Hz, 1H), 5.21(dd, J=2.5Hz and 6.3Hz, 1H), |
| | 7.31(d, J=8.8Hz, 2H), 7.98(d, J=8.8Hz, |
| | 2H), 8.16(dd, J=1.2 and 2.7Hz, 1H), |
| | 8.29(d, J=2.7Hz, 1H), 8.33(br, 1H), 8.40-8.50 |
| | (m, 2H). |
| 29-6 | (+)-6-(4-Pentyloxybenzenesulfonyl)-5,6,7,8- |
| | tetrahydropyrido[3,4-b]- |
| | pyrazine-7-carboxylic acid |
| | ¹H-NMR(250MHz, CDCl₃)δ; |
| | 0.94(t, J=6.8Hz, 3H), 1.30-1.60(m, 4H), 1.70-1.90 |
| | (m, 2H), 3.30-3.50(m, 2H), 3.99(t, J=6.6Hz, 2H), |
| | 4.62(d, J=17.1Hz, 1H), 4.88(d, |
| | J=17.1Hz, 1H), 5.10-5.30(m, 1H), 6.95(d, |
| | J=8.8Hz, 2H), 7.20-7.40(m, 5H), 7.79(d, |
| | J=8.8Hz, 2H), 8.30-8.50(m, 2H). |
| 29-7 | (+)-6-(4-Methylthiobenzenesulfonyl)- |
| | 5,6,7,8-tetrahydropyrido[3,4-b]- |
| | pyrazine-7-carboxylic acid |
| | ¹H-NMR(250MHz, CDCl₃)δ; |
| | 2.50(s, 3H), 3.30-3.50(m, 2H), 4.63(d, J=17.1Hz, 1 |
| | H), 4.89(d, J=17.1Hz, 1H), 5.21(dd, J=5.4 |
| | and 3.3Hz, 1H), 7.32(d, J=8.7Hz, 2H), |
| | 7.76(d, J=8.7Hz, 2H), 8.30(d, J=2.5Hz, |
| | 1H), 8.41(d, J=2.5Hz, 1H). |
| 29-8 | (+)-6-(4-Acetylbenzenesulfonyl)-5,6,7,8- |
| | tetrahydropyrido[3,4-b]pyrazine-7- |
| | carboxylic acid |
| | ¹H-NMR(250MHz, CDCl₃)δ; |
| | 2.65(s, 3H), 3.40-3.60(m, 2H), 4.58(d, J=17.0Hz, 1 |
| | H), 4.94(d, J=17.0Hz, 1H), 5.20-5.30(m, 1H), |
| | 7.97(d, J=8.5Hz, 2H), 8.07(d, J=8.5Hz, |
| | 2H), 8.35(d, J=2.6Hz, 1H), 8.40(d, J=2.6Hz, 1H). |
| 29-9 | (+)-6-[4-(3-phenylpropoxy)benzenesulfonyl]-5,6,7,8- |
| | tetrahydropyrido[3,4-b]- |
| | pyrazine-7-carboxylic acid |
| | ¹H-NMR(250MHz, CDCl₃)δ; |
| | 2.00-2.20(m, 2H), 2.80(t, J=6.3Hz, 2H), 3.20-3.80 |
| | (m, 2H), 3.99(t, J=6.3Hz, 2H), 4.62(d, J=17.1Hz, |
| | 1H), 4.87(d, J=17.1Hz, 1H), 5.21 |
| | (dd, J=3.4 and 5.4Hz, 1H), 6.93(d, J=8.8Hz, |
| | 2H), 6.90-7.30(m, 5H), 7.79(d, J=8.8Hz, |
| | 2H), 8.33(d, J=2.5Hz, 1H), 8.38(d, J=2.5Hz, 1H). |
| 29-10 | (+)-5,6,7,8-tetrahydropyrido[3,4-b]pyrazine- |
| | 6-(thiophen-2-sulfonyl)-7- |
| | carboxylic acid |
| | ¹H-NMR(250MHz, CDCl₃-D₂O)δ; |
| | 3.44(d, J=4.3Hz, 2H), 4.73(d, J=17.2Hz, 1H), |
| | 4.95(d, J=17.2Hz, 1H), 5.22(t, J=4.4Hz, 1H), |
| | 7.00-7.20(m, 1H), 7.60-7.70(m, 2H), |
| | 8.37(d, J=2.6Hz, 1H), 8.44(d, J=2.6Hz, 1H). |
| 29-11 | (+)-6-(2-trans-phenylethenesulfonyl)- |
| | 5,6,7,8-tetrahydropyrido[3,4-b]- |
| | pyrazine-7-carboxylic acid |
| | ¹H-NMR(250MHz, CDCl₃)δ; |
| | 3.54(d, J=3.7Hz, 2H), 4.68(d, J=17.0Hz, 1H), 4.87 |
| | (d, J=17.0Hz, 1H), 5.23(dd, J=3.7 and 4.9Hz, 1H), |
| | 6.85(d, J=16.0Hz, 1H), 7.30-7.60 |
| | (m, 5H), 7.66(d, J=16.0Hz, 1H), |
| | 8.40(d, J=2.6Hz, 1H), 8.45(d, J=2.6Hz, 1H). |
| 29-12 | (+)-6-[4-(3- |
| | methanesulfonylpropoxy)benzenesulfonyl]-5,6,7,8-tetra- |
| | hydropyrido[3,4-b]pyrazine-7-carboxylic acid |
| | ¹H-NMR(250MHz, DMSO-d₆)δ; |
| | 2.00-2.30(m, 2H), 3.01(s, 3H), 3.10-3.40(m, 2 |
| | H), 4.14(t, J=6.2Hz, 2H), 4.49(d, J=17.0Hz, |
| | 1H), 4.67(d, J=17.0Hz, 1H), 4.99(dd, |
| | J=3.3 and 5.6Hz, 1H), 7.06(d, J=8.9Hz, 2H), |
| | 7.79(d, J=8.9Hz, 2H), 8.40-8.50(m, 2 |
| | H). |
| 29-13 | (+)-6-[5- |
| | (2-methoxyethyl)thiophenesulfonyl]-5,6,7,8- |
| | tetrahydropyrido[3,4-b]- |
| | pyrazine-7-carboxylic acid |
| | ¹H-NMR(250MHz, CDCl₃)δ; |
| | 3.06(t, J=6.1Hz, 2H), 3.36(s, 3H), 3.43(d, J=4.3Hz, |
| | 2H), 3.61(t, J=6.1Hz, 2H), 4.76(d, J=17.1Hz, 1H), |
| | 4.93(d, J=17.1Hz, 1H), 5.21(t, |
| | J=4.3Hz, 1H), 6.84(d, J=3.8Hz, 1H), 7.50(d, |
| | J=3.8Hz, 1H), 7.83(br s, 1H), 8.37(d,N |
| | J=2.5Hz, 1H), 8.44(d, J=2.5Hz, 1H). |
| 29-14 | (+)-6-[2-trans-(4- |
| | methoxyphenyl)ethenesulfonyl]-5,6,7,8- |
| | tetrahydropyrido[3,4-b]- |
| | pyrazine-7-carboxylic acid |
| | ¹H-NMR(250MHz, DMSO-d₆)δ; |
| | 3.15-3.35(m, 1H), 3.35-3.55(m, 1H), 3.78(s, 3 |
| | H), 4.55(d, J=16.9Hz, 1H), 4.65(d, J=16.9Hz, |
| | 1H), 4.90-5.00(m, 1H), 6.97(d, J=8.8Hz, |
| | 2H), 7.17(d, J=15.4Hz, 1H), 7.42(d, J=15.4Hz, |
| | 1H), 7.62(d, J=8.8Hz, 2H), 8.40-8.50 |
| | (m, 2H). |

### Reference Example 30

(±)-N-Benzyloxy-6-[4-(2-hydroxyethoxy)benzenesulfonyl]-5,6,7,8-tetrahydropyrido[3,4-b]pyrazine-7-carboxamide

(+)-N-Benzyloxy-6-[4-(2-acetoxyethoxy)benzenesulfonyl]-5,6,7,8-tetrahydropyrido[3,4-b]pyrazine-7-carboxamide (206 mg) was dissolved in a mixture of methanol (4 ml) and 1,4-dioxane (6 ml) and thereto was added 28% sodium hydroxide/methanol solution (0.1 ml) and the mixture was stirred at room temperature for an hour. The reaction mixture was diluted with saturated saline solution and extracted with ethyl acetate. The ethyl acetate layer was washed with saturated saline solution and dried over magnesium hydroxide. After removing magnesium hydroxide, the solvent was distilled off under reduced pressure and finally the residue was purified by a silica gel column chromatography of medium pressure (mobile phase; ethyl acetate/methanol=20/1) to give the title compound (150 mg) as colorless materials.

¹H-NMR(250 MHz, CDCl₃) δ; 1.99 (br, 1H), 2.85 (dd, J=17.8 and 6.9 Hz, 1H), 3.46 (d, J=17. 8 Hz, 1H), 3.90-4.20 (m, 4H), 4.40 (d, J=17.8 Hz, 1H), 4.70-5.00 (m, 4H), 6.89 (d, J=9.0 Hz, 2H), 7.30-7.50 (m, 5H), 7.67 (d, J=9.0 Hz, 2H), 8.35 (s, 2H), 9.12 (br s, 1H).

### Reference Example 31

(+)-N-Benzyloxy-6-(4-hydroxybenzenesulfonyl)-5,6,7,8-tetrahydropyrido [3,4-b]pyrazine-7-carboxamide

(+)-N-Benzyloxy-6-(4-pivaloyloxybenzenesulfonyl)-5,6,7,8-tetrahydropyrido[3,4-b]pyrazine-7-carboxamide (350 mg) was dissolved in 1,4-dioxane (5 ml) and thereto was added 1N sodium hydroxide aqueous solution (2.0 ml), and the mixture was stirred at room temperature for 2 hours. The reaction mixture was diluted with 10% citric acid aqueous solution and extracted with ethyl acetate. The ethyl acetate layer was washed with saturated saline solution and dried over magnesium sulfate. After removing magnesium hydroxide, the solvent was distilled off under reduced pressure and finally the residue was purified by a silica gel column chromatography of medium pressure (mobile phase; n-hexane/ethyl acetate=1/2) to give the title compound (210 mg) as colorless solids.

¹H-NMR(250 MHz, CDCl₃) δ; 2.80-3.00 (m, 1H), 3.40-3.60 (m, 1H), 4.40 (d, J=17.1 Hz, 1H), 4.70-5.00 (m, 4H), 6.81 (d, J=8.5 Hz, 2H), 7.30-7.50 (m, 5H), 7.62 (d, J=8.5 Hz, 2H), 8.37 (s, 2H), 9.11 (s, 1H).

### Reference Example 32

7,8-cis-5,8-Dimethyl-6-(4-methoxybenzenesulfonyl)-5,6,7,8-tetrahydropyrido[3,4-b]pyrazine-7-carboxylic Acid

(a) 2,3-Bis(1-chloroethyl)pyrazine

2,3-diethylpyrazine (9.86 g) was dissolved in carbon tetrachloride (100 ml) and thereto were added N-chlorosuccinimide (19.3 g) and benzoyl peroxide (300 mg), and the mixture was stirred at refluxing temperature for a day. The precipitated solids were removed by filtration and the solvent was distilled off in vacuo. The residue was purified by a silica gel column chromatography (mobile phase; cyclohexane/ethyl acetate=10/1) to give the title compound (4.34 g) as pale brown oil, which were a mixture of two diastereoisomers (the diastereoisomer A and B).

The Diastereoisomer A;

¹H-NMR(250 MHz, CDCl₃) δ; 1.97 (d, J=6.6 Hz, 6H), 5.56 (q, J=6.6 Hz, 2H), 8.55 (s, 2H).

The Diastereoisomer B;

¹H-NMR(250 MHz, CDCl₃) δ; 1.97 (d, J=6.6 Hz, 6H), 5.45 (q, J=6.6 Hz, 2H), 8.57 (d, J=2.4 Hz, 1H), 8.60 (d, J=2.4 Hz, 1H).

(b) Ethyl 5,8-dimethyl-5,6,7,8-tetrahydropyrido[3,4-b]pyrazine-7-carboxylate

To a mixture of 2,3-bis(1-chloroethyl)pyrazine (1.00 g), N-(diphenylmethylene) glycine ethyl ester (1.30 g), benzyltriethylammonium chloride (112 mg) and acetonitrile (10 ml), was added powdered potassium hydroxide (0.15 g) at 0° C. and the mixture was stirred at room temperature overnight. The reaction mixture was adjusted to pH 7 by adding 10% citric acid solution and was extracted with ethyl acetate. The ethyl acetate layer was washed with water and saturated saline solution successively, and dried over magnesium sulfate. After magnesium sulfate was removed, the solvent was distilled off in vacuo and the residue was purified by a silica gel column chromatography of medium pressure (mobile phase; cyclohexane/ethyl acetate =5/1 to 3/1) to give ethyl 3-[3-(1-chloroethyl)pyrazine-2-yl]-2-(diphenylmethylene)aminobutanoate (300 mg).

A mixture of ethyl 3-[3-(1-chloroethyl)pyrazine-2-yl]-2-(diphenylmethylene) aminobutanoate (300 mg), 1N hydrochloric acid (5 ml) and diethylether (10 ml) was stirred at room temperature for 3 hours. To the aqueous layer which was separated from the reaction mixture, was added sodium bicarbonate and the mixture was stirred at room temperature overnight. The reaction mixture was diluted with water and extracted with ethyl acetate. The ethyl acetate layer was washed with water and saturated saline solution successively and dried over magnesium sulfate. Magnesium sulfate was removed and the solvent was distilled off in vacuo. The residue was dissolved in DMF (5 ml) and thereto was added potassium carbonate (75 mg), and the mixture was stirred at room temperature for 2 days. The reaction mixture was diluted with water and extracted with ethyl acetate. The ethyl acetate layer was washed with water and saturated saline solution successively, and dried over magnesium sulfate. After magnesium sulfate was removed, the solvent was distilled off in vacuo and the residue was finally purified by a silica gel column chromatography of medium pressure (mobile phase; chloroform/methanol=100/1 to 50/1) to give the title compound (51 mg) as brown oil. The oil was a mixture of two diastereoisomers in which the stereochemistry of 7- and 8-positions were different (the ratio of the diastereoisomers; 7,8-cis isomer/7,8-trans isomer=1/3).

7,8-cis Isomer;

¹H-NMR(250 MHz, CDCl₃) δ; 1.27 (d, J=7.1 Hz, 3H), 1.30 (t, J=7.1 Hz, 3H), 1.47 (d, J=6.9 Hz, 3H), 3.37 (dq, J=4.0 and 7.1 Hz, 1H), 4.10 (d, J=4.0 Hz, 1H), 4.20-4.30 (m, 2H), 4.36 (q, J=6.9H z, 1H), 8.30-8.40 (m, 2H).

7,8-trans Isomer;

¹H-NMR(250 MHz, CDCl₃) δ; 1.32 (t, J=7.0 Hz, 3H), 1.39 (d, J=7.0 Hz, 3H), 1.54 (d, J=6.7 Hz, 3H), 3.20-3.30 (m, 1H), 3.49 (d, J=10.3 Hz, 1H), 4.18 (d, J=7.0 Hz, 1H), 4.26 (dq, J=1.8 and 7.0 Hz, 2H), 8.34 (dd, J=0.9 and 2.5 Hz, 1H), 8.39 (dd, J=0.9 and 2.5 Hz, 1H).

(c) Ethyl 6-(4-methoxybenzenesulfonyl)-5,8-dimethyl-5,6,7,8-tetrahydropyrido[3,4-b]pyrazine-7-carboxylate

A mixture of ethyl 5,8-dimethyl-5,6,7,8-tetrahydropyrido[3,4-b]pyrazine-7-carboxylate (7,8-cis/7,8-trans=1/3) (51 mg), 4-methoxybenzenesulfonyl chloride (45 mg), triethylamine (37 μl) and acetonitrile(5 ml) was stirred at 50° C. overnight, and further at 70° C. for 24 hours. The reaction mixture was adjusted to pH 7 by adding 10% citric acid solution and extracted with ethyl acetate. The ethyl acetate layer was washed with water and saturated saline solution successively, and dried over magnesium sulfate. After magnesium sulfate was removed, the residue was distilled off in vacuo and the residue was finally purified by a silica gel column chromatography of medium pressure (mobile phase; n-hexane/ethyl acetate=3/1) to give the title compound (4.0 mg) as brown oil. The oil was a mixture of two diastereoisomers in which the stereochemistry of 7- and 8-positions were different (the ratio of the diastereoisomers; 7,8-cis isomer/7,8-trans isomer=10/1).

7,8-cis Isomer

¹H-NMR(250 MHz, CDCl₃) δ; 1.17 (t, J=7.2 Hz, 3H), 1.32 (d, J=7.0 Hz, 3H), 1.38 (d, J=7.2 Hz, 3H), 3.66 (dq, J=2.2 and 7.2 Hz, 1H), 3.87 (s, 3H), 3.90-4.20 (m, 2H), 4.96 (d, J=2.2 Hz, 1H), 5.19 (q, J=7.0 Hz, 1H), 6.97 (d, J=9.0 Hz, 2H), 7.92 (d, J=9.0 Hz, 2H), 8.37 (d, J=2.4 Hz, 1H), 8.41 (dd, J=0.6 and 2.5 Hz, 1H).

7,8-trans Isomer

¹H-NMR(250 MHz, CDCl₃) δ; 0.82 (t, J=7.0 Hz, 3H), 1.46 (d, J=7.0 Hz, 3H), 1.70 (d, J=6.8 Hz, 3H), 3.30-3.60 (m, 1H), 3.87 (s, 3H), 3.90-4.20 (m, 2H), 4.78 (dd, J=0.9 and 5.2 Hz, 1H), 5.10-5. 20 (m, 1H), 7.07 (d, J=9.0 Hz, 2H), 7.80 (d, J=9.0 Hz, 2H), 8.33 (dd, J=0.9 and 2.7 Hz, 1H), 8.30-8.40 (m, 1H).

(d) 7,8-cis-5,8-Dimethyl-6-(4-methoxybenzenesulfonyl)-5,6,7,8-tetrahydropyrido[3,4-b]pyrazine-7-carboxylic Acid

A mixture of ethyl 6-(4-methoxybenzenesulfonyl)-5,8-dimethyl-5,6,7,8-tetrahydropyrido[3,4-b]pyrazine-7-carboxylate (7,8-cis/7,8-trans=10/1) (4.0 mg), THF (5 ml) and 1N sodium hydroxide solution(200 μl) was stirred at 50° C. for 3 hours. The reaction mixture was diluted with water and washed with ether. Then it was adjusted to pH 4 by adding 10% citric acid solution and extracted with ethyl acetate.

The ethyl acetate layer was washed with water and saturated saline solution successively, and dried over magnesium sulfate. After magnesium sulfate was removed, the residue was distilled off in vacuo to give the title compound (3.0 mg) as colorless solids. The stereochemistry of 7- and 8-positions in the product was cis.

¹H-NMR(250 MHz, CDCl₃)δ; 1.25 (d, J=7.1 Hz, 3H), 1.45 (d, J=7.1 Hz, 3H), 3.68 (dq, J=2.5 and 7.1 Hz, 1H), 3.86 (s, 3H), 4.83 (d, J=2.5 Hz, 1H), 5.22 (q, J=6.8 Hz, 1H), 6.95 (d, J=9.0 Hz, 2H), 7.88 (d, J=9.0 Hz, 2H), 8.39 (d, J=2.5 Hz, 1H), 8.41 (d, J=2.5 Hz, 1H).

### Reference Example 33

(±)-2,3-dimethyl-6-(4-methoxybenzenesulfonyl)-5,6,7,8-tetrahydropyrido[3,4-b]pyrazine-7-carboxylic Acid

(a) 2,3-Dimethyl-5,6-bis(hydroxymethyl)pyrazine

To a mixture of 2,3-diamino-1,4-butanediol (1.2 g), potassium hydroxide (560 mg) and methanol (50 ml), was added 2,3-butanedione (860 mg) and the mixture was stirred at room temperature for an hour. The oxygen gas was blown into the reaction mixture with stirring for 3 hours, and then the mixture was adjusted to pH 7 by adding 5N hydrochloric acid. The reaction mixture was condensed and the resulting residue was purified by a silica gel column chromatography of medium pressure (mobile phase; chloroform/methanol=10/1) to give the title compound (520 mg) as brown solids.

¹H-NMR(250 MHz, CDCl₃) δ;2.57 (s, 6H), 4.07 (br s, 2H), 4.69 (s, 4H).

(b) 2,3-Dimethyl-5,6-bis(chloromethyl)pyrazine

2,3-Dimethyl-5,6-bis(hydroxymethyl)pyrazine (0.52 g) was dissolved in DMF (10 ml) and thereto was added phosphorus trichloride (0.47 g) dropwise under ice-cooling, and the mixture was stirred at room temperature for an hour. The reaction mixture was diluted with water and extracted with ethyl acetate. The ethyl acetate layer was washed with water and saturated saline solution successively, and dried over magnesium sulfate. After magnesium sulfate was removed, the residue was distilled off in vacuo to give the title compound (0.42 g)

¹H-NMR(250 MHz, CDCl₃) δ;2.57 (s, 6H), 4.79 (s, 4H).

(c) Ethyl 6-acetyl-2,3-dimethyl-6-ethoxycarbonyl—5,6,7,8-tetrahydropyrido[3,4-b]pyrazine-7-carboxylate

2,3-Dimethyl-5,6-bis(chloromethyl)pyrazine (0.42 g) was dissolved in acetonitrile (50 ml) and thereto were added diethyl acetamidomalonate (0.46 g) and cesium carbonate (2.0 g), and the mixture was stirred at refluxing temperature for 3 hours. After the reaction mixture was cooled to room temperature, insoluble materials were removed by filtration. The solvent of the filtrate was distilled off in vacuo and the residue was purified by a silica gel column chromatography of 30 medium pressure (mobile phase; chloroform/methanol=100/1) to give the title compound (600 mg) as blown oil.

¹H-NMR(250 MHz, CDCl₃) δ; 1.10-1.40 (m, 6H), 1.96 (s, 3H), 2.51 (s, 3H), 2.53 (s, 3H), 3.62 (s, 2H), 4.10-4.40 (m, 4H), 4.77 (s, 2H).

(d) (±)-2,3-Dimethyl-6-(4-methoxybenzenesulfonyl)-5,6,7,8-tetrahydropyrido[3,4-b]pyrazine-7-carboxylic Acid

Ethyl 6-acetyl-2,3-dimethyl-6-ethoxycarbonyl-5,6,7,8-tetrahydropyrido[3,4-b]pyrazine-7-carboxylate (600 mg) was refluxed in 6N hydrochloric acid for 6 hours. The reaction mixture was cooled to room temperature and the solvent was distilled off in vacuo.

The resulting residue was dissolved in a mixed solvent of water (20 ml) and 1,4-dioxane (20 ml) and thereto were added triethylamine (850 mg) at room temperature and then 4-methoxybenzenesulfonyl chloride (530 mg) under ice-cooling, and the mixture was stirred under ice-cooling for an hour. The mixture was diluted with 1N hydrochloric acid and extracted with ethyl acetate. The ethyl acetate layer was washed with water and dried over magnesium sulfate. After magnesium sulfate was removed, the solvent was distilled off in vacuo and the residue was finally purified by a silica gel column chromatography of medium pressure (mobile phase; n-hexane/ethyl acetate=100/1) to give the title compond (170 mg) as yellow oil.

¹H-NMR(250 MHz, CDCl₃) δ;2.41 (s, 6H), 3.10-3.40 (m, 2H), 3.84 (s, 3H), 4.49 (d, J=16.7 Hz, 1H), 4.74 (d, J=16.7 Hz, 1H), 5.13 (dd, J=2.2 and 6.2 Hz, 1H), 6.94 (d, J=8.9 Hz, 2H), 7.78 (d, J=8.9 Hz, 2H), 8.67 (br s, 1H).

### Reference Example 34

(+)-6-(4-Ethynylbenzenesulfonyl)-5,6,7,8-tetrahydropyrido[3,4-b]pyrazine-7-carboxylic Acid

(a) Methyl (+)-6-(4-bromobenzenesulfonyl)-5,6,7,8-tetrahydropyrido[3,4-b]pyrazine-7-carboxylate

Methyl (+)-5,6,7,8-tetrahydropyrido[3,4-b]pyrazine-7-carboxylate hydrochloride (1.15 g) was dissolved in a mixed solvent of water (20 ml) and 1,4-dioxane (20 ml) and thereto were added triethylamine (1.62 g) and 4-bromobenzenesulfonyl chloride (1.77 g), and the mixture was stirred at room temperature for 15 hours. To the reaction mixture was added 10% citric acid solution (100 ml), and the products were extracted with ethyl acetate. The ethyl acetate layer was washed with 10% citric acid solution, saturated sodium bicarbonate solution and water successively and dried over magnesium sulfate. After magnesium sulfate was removed, the solvent was distilled off in vacuo and the residue was finally purified by a silica gel column chromatography of medium pressure (mobile phase; n-hexane/ethyl acetate=1/2) to give the title compound (0.95 g) as pale yellow oil.

¹H-NMR(250 MHz, CDCl₃) δ;3.40-3.50 (m, 5H), 4.53 (d, J=16.9 Hz, 1H), 4.92 (d, J=16.9 Hz, 1H), 5.15-5.25 (m, 1H), 7.60-7.80 (m, 4H), 8.35-8.45 (m, 2H).

(b) Methyl (+)-6-(4-trimethylsilylethynylbenzenesulfonyl)-5,6,7,8-tetrahydropyrido-[3,4-b]pyrazine-7-carboxylate

Methyl (+)-6-(4-bromobenzenesulfonyl)-5,6,7,8-tetrahydropyrido[3,4-b]pyrazine-7-carboxylate (280 mg) was dissolved in acetonitrile (1 ml) and thereto were added triethylamine (1 ml), 10% Pd—C (36 mg), triphenylphosphine (28 mg), cuprus iodide (6.5 mg) and trimethylsilylacetylene (80 mg), and the mixture was stirred at refluxing temperature for 2.5 hours.

After the reaction mixture was cooled to room temperature, methanol (20 ml) was added and insoluble materials were removed by filtration. The solvent of the filtrate was distilled off in vacuo and the residue was purified by a silica gel column chromatography of medium pressure (mobile phase; n-hexane/ethyl acetate=2/1) to give the title compound (190 mg) as crystals.

¹H-NMR(250 MHz, CDCl₃) δ; 0.21 (s, 9H), 3.30-3.45 (m, 5H), 4.48 (d, J=16.8 Hz, 1H), 4.89 (d, J=16.8 Hz, 1H), 5.10-5.20 (m, 1H), 7.54 (d, J=8.5 Hz, 2H), 7.75 (d, J=8.5 Hz, 2H), 8.30-8.40 (m, 2H).

(c) (+)-6-(4-Ethynylbenzenesulfonyl)-5,6,7,8-tetrahydropyrido[3,4-b]pyrazine-7-carboxylic Acid

Methyl (+)-6-(4-trimethylsilylethynylbenzenesulfonyl)-5,6,7,8-tetrahydropyrido[3,4-b]pyrazine-7-carboxylate (190 mg) was dissolved in 1,4-dioxane (1.5 ml) and thereto was added 1N potassium hydroxide solution (2.3 ml), and the mixture was stirred at room temperature for 2 hours. The reaction mixture was diluted with water (30 ml) and adjusted to pH 3 by adding 10% citric acid solution. The products were extracted with ethyl acetate and the ethyl acetate layer was washed with saturated saline solution, and dried over magnesium sulfate. After magnesium sulfate was removed, the solvent was distilled off in vacuo and the residue was finally purified by a silica gel column chromatography of medium pressure (mobile phase; chloroform/methanol=10/1) to give the title compound (80 mg) as yellow solids.

¹H-NMR(250 MHz, DMSO-d₆) δ;3.19 (dd, J=2,3 and 17.4 Hz, 1H), 3.25-3.40 (m, 1H), 4.48 (d, J=17.0 Hz, 1H), 4.51 (s, 1H), 4.74 (d, J=17.0 Hz, 1H), 5.08 (dd, J=2.3 and 6.4 Hz, 1H), 7.65 (d, J=8.3 Hz, 2H), 7.87 (d, J=8.3 Hz, 2H), 8.40-8.50 (m, 2H).

What is claimed is:

1. A keratinocyte-proliferation inhibitor comprising as active Ingredient a compound having an activity of inhibiting the solubilization of heparin-binding EGF-like growth factor bound to cell membranes.

2. The keratinocyte-proliferation suppresor according to claim 1, comprising as active ingredient a compound of the general formula (I): 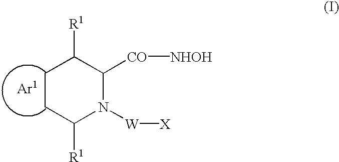
or a pharmaceutically acceptable salt thereof, wherein
Ar¹ is any cyclic structure selected from the following formulas (A) to (C): 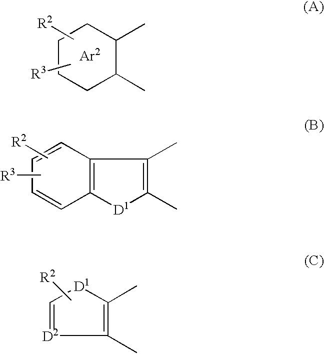
wherein Ar² is a 6-membered aromatic ring which may include 1 to 2 nitrogen atoms, D¹ is —(CH₂)ₙ— (n is integer of 1 to 3), oxygen atom, sulfur atom, —S(O)—, —S(O)₂— or —NR⁴— (R⁴ is hydrogen atom, C₁-C₈ alkyl or C₁-C₈ acyl), D² is —(CH)— or nitrogen atom, R² and R³ may be the same or different, and are hydrogen atom, hydroxyl, C₁-C₈ alkyl, C₂-C₈ alkenyl, C₂-C₈ alkynyl, halogen atom or —NR⁵R⁶ (R⁵ and R⁶ may be the same or different, and are hydrogen atom, C₁-C₈ alkyl, C₁-C₈ acyl, carbamoyl or C₁-C₄ alkylcarbamoyl), wherein the said C₁-C₈ alkyl, C₂-C₈ alkenyl and C₂-C₈ alkynyl may be substituted by halogen atom, heterocycle, C₁-C₄ alkoxy, C₁-C₄ acyl, C₁-C₄ acyloxy, heteroaryloxy, C₁-C₄ alkylthio, phenyl, phenoxy or NR⁷R⁸— (R⁷ and R⁸ may be the same or different, and are hydrogen atom, C₁-C₈ alkyl or C₁-C₈ acyl); R¹ is hydrogen atom or methyl; W is divalent atomic group of the following formula (IIa) or —(IIb): 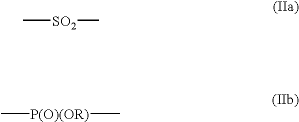
wherein R is hydrogen atom, C₁-C₈ alkyl or phenyl, wherein the said C₁-C₈ alkyl and phenyl may be substituted by halogen atom, hydroxyl, nitrile, C₁-C₄alkyloxycarbonyl, carboxyl, carbamoyl, C₁-C₄ alkylcarbamoyl, C₂-C₄ alkenyl, C₂-C₄ alkynyl, heterocycle, C₁-C₄ alkoxy, C₁-C₄ acyl, C₁-C₄ acyloxy, heteroaryloxy, C₁-C₄ alkylthio, phenyl, biphenyl, phenoxy, C₁-C₄ alkylsulfonyl, arylsulfonyl, arylaminosulfonyl or —NR⁹R¹⁰ (R⁹ and R¹⁰ may be the same or different, and are hydrogen atom, C₁-C₈ alkyl, C₁-C₈ acyl, carbamoyl or C₁-C₄ alkylcarbamoyl); X is atomic group of the following formula: 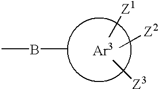
wherein cyclic structure Ar³ is benzene ring or 5- or 6-membered heteroaromatic ring including one oxygen atom, one sulfur atom or 1 to 2 nitrogen atoms, B is covalent bond, —(CH₂)_{q}— (q is integer of 1 to 4), CH═CH—, —C(O)—, —CH(OH)— or NR¹¹-, R¹¹ is hydrogen atom, C₁-C₈ alkyl, C₂-C₈ alkenyl or C₂-C₈ alkynyl, and the said C₁-C₈ alkyl, C₂-C₈ alkenyl and C₂-C₈ alkynyl may be substituted by halogen atom, heterocycle, C₁-C₈ alkoxy, C₁-C₈ acyl, C₁-C₈ acyloxy, heteroaryloxy, C₁-C₈ alkylthio, phenyl, phenoxy or —NR¹²R¹³ (R¹² and R¹³ may be the same or different, and are hydrogen atom, C₁-C₈ alkyl, C₁-C₈ acyl, carbamoyl or C₁-C₄ alkylcarbamoyl); Z¹, Z² and Z³ may be the same or different, and are hydrogen atom, C₁-C₈ alkyl, C₂-C₈ alkenyl, C₂-C₈ alkynyl, phenyl, heterocycle, halogenatom, hydroxyl, C₁-C₈alkoxy, C₁-C₈ acyl, C₁-C₈ acyloxy, phenoxy, heteroaryloxy, C₁-C₈ alkylthio or —NR¹⁴R¹⁵ (R¹⁴ and R¹⁵ may be the same or different, and are hydrogen atom, C₁-C₈alkyl, C₁-C₈acyl, carbamoyl or C₁-C₄alkylcarbamoyl), wherein the said C₁-C₈ alkyl, phenyl, heterocycle, C₂-C₈ alkenyl, C₂-C₈ alkynyl, C₁-C₈ alkoxy, C₁-C₈ acyl, C₁-C₈ acyloxy, phenoxy, heteroaryloxy and C₁-C₈ alkylthio may be substituted by halogen atom, heterocycle, C₁-C₈ alkoxy, C₁-C₄ acyl, C₁-C₄ alkylsulfonyl, cyano, C₁-C₄ alkoxycarbonyl, hydroxyl, C₁-C₈ acyloxy, heteroaryloxy, C₁-C₈ alkylthio, phenyl, phenoxy or amino, or Z¹ and Z² may be combined to represent divalent atomic group —O—CH₂—O—.

3. The keratinocyte-proliferation suppresor according to claim 1 or 2, comprising as active ingredient a compound of the general formula (Ia): 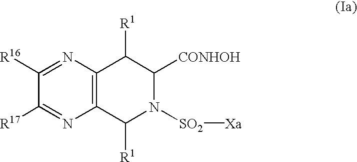
or a pharmaceutically acceptable salt thereof, wherein
R¹ is hydrogen atom or methyl, R¹⁶ and R¹⁷ may be the same or different, and are hydrogen atom or C₁-C₄ alkyl, Xa is atomic group of the following formula (D), (E), (F) or (G): 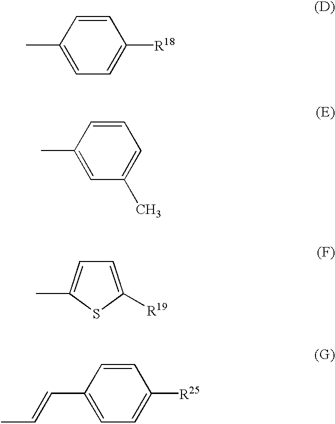
wherein R¹⁸ is hydrogen atom, halogen atom, hydroxyl, amino, C₁-C₈ alkyl, vinyl, ethynyl, phenyl, C₁-C₈ alkylthio, phenoxy, 4-aminophenoxy, heteroaryloxy, C₁-C₈ acyloxy, C₁-C₈ acyl or C₁-C₈ alkoxy, wherein the said C₁-C₈ alkoxy may be substituted by 2-propynyl, 2-butynyl, phenyl, halogen, C₁-C₄ alkoxy, C₁-C₄ alkylthio, C₁-C₄ alkylsulfonyl, cyano, C₁-C₄ alkoxycarbonyl, C₁-C₄ acyloxy or hydroxyl; R¹⁹ is hydrogen atom or C₁-C₈ alkyl, wherein the said C₁-C₈ alkyl may be substituted by C₁-C₄ alkoxy, and R²⁵ is hydrogen atom or C₁-C₈ alkoxy.

4. The keratinocyte-proliferation suppresor according to claim 1 or 2, comprising as active ingredient a compound of the general formula (Ib): 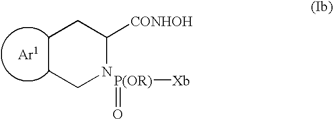
or a pharmaceutically acceptable salt thereof, wherein
Ar¹ is any cyclic structure selected from the following formulas (A) to (C): 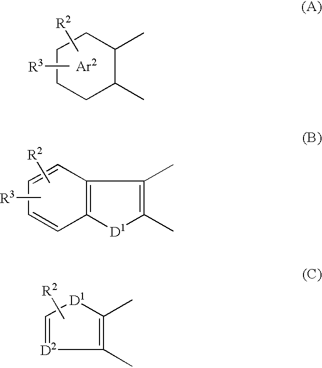
wherein Ar² is 6-membered aromatic ring which may include 1 to 2 nitrogen atoms, D¹ is —(CH₂)n— (n is integer of 1 to 3), oxygen atom, sulfur atom, —S(O)—, —S(O)₂— or NR⁴— (R⁴ is hydrogen atom, hydroxyl, C₁-C₈ alkyl or C₁-C₈ acyl), D² is —(CH)— or nitrogen atom, R² and R³, may be the same or different, and are, hydrogen atom, C₁-C₈ alkyl, C₂-C₈ alkenyl, C₂-C₈ alkynyl, halogen atom, —NR⁵R⁶ (R⁵ and R⁶ may be the same or different, and are hydrogen atom, C₁-C₈alkyl, C₁-C₈acyl, carbamoyl or C₁-C₄alkylcarbamoyl), wherein the said C₁-C₈ alkyl, C₂-C₈ alkenyl and C₂-C₈ alkynyl may be substituted by halogen atom, heterocycle, C₁-C₄ alkoxy, C₁-C₄ acyl, C₁-C₄ acyloxy, heteroaryloxy, C₁-C₄ alkylthio, phenyl, phenoxy or NR⁷R⁸—(R⁷ and R⁸ may be the same or different, and are hydrogen atom, C₁-C₈ alkyl or C₁-C₈ acyl);
R is hydrogen atom, C₁-C₈ alkyl or phenyl, wherein the said CL-C₈ alkyl and phenyl may be substituted by halogen atom, hydroxyl, nitrile, C₁-C₄alkyloxycarbonyl, carboxyl, carbamoyl, C₁-C₄ alkylcarbamoyl, C₂-C₄ alkenyl, C₂-C₄ alkynyl, heterocycle, C₁-C₄ alkoxy, C₁-C₄ acyl, C₁-C₄ acyloxy, heteroaryloxy, C₁-C₄ alkylthio, phenyl, biphenyl, phenoxy, C₁-C₄ alkylsulfonyl, arylsulfonyl, arylaminosulfonyl or NR⁹R¹⁰ (R⁹ and R¹⁰ may be the same or different, and are hydrogen atom, C₁-C₈ alkyl, C₁-C₈ acyl, carbamoyl or C₁-C₄ alkylcarbamoyl); Xb is atomic group of the following formula: 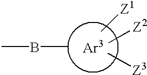
wherein cyclic structure Ar³ is benzene ring or 5- or 6-membered heteroaromatic ring including one oxygen atom, one sulfur atom or 1 to 2 nitrogen atoms, B is covalent bond, —(CH₂)_{q}— (q is integer of 1 to 4), —CH═CH—, —C(O)—, —CH(OH)— or N¹¹—, R¹¹ is hydrogen atom, C₁-C₈alkyl, C₂-C₈alkenylor C₂-C₈alkynyl, wherein the said C₁-C₈ alkyl, C₂-C₈ alkenyl and C₂-C₈ alkynyl may be substituted by halogen atom, heterocycle, C₁-C₈ alkoxy, C₁-C₈ acyl, C₁-C₈ acyloxy, heteroaryloxy, C₁-C₈ alkylthio, phenyl, phenoxy or NR¹²R¹³ (R¹² and R¹³ may be the same or different, and are hydrogen atom, C₁-C₈ alkyl, C₁-C₈ acyl, carbamoyl or C₁-C₄ alkylcarbamoyl); Z¹, Z² and Z³ may be the same or different, and are hydrogen atom, C₁-C₈ alkyl, C₂-C₈ alkenyl, C₂-C₈ alkynyl, phenyl, heterocycle, halogen atom, hydroxyl, C₁-C₈ alkoxy, C₁-C₈ acyl, C₁-C₈ acyloxy, phenoxy, heteroaryloxy, C₁-C₈ alkylthio or —NR¹⁴R¹⁵ (R¹⁴ and R¹⁵ may be the same or different, and are hydrogen atom, C₁-C₈alkyl, C₁-C₈acyl, carbamoyl or C₁-C₄alkylcarbamoyl), wherein the said C₁-C₈ alkyl, phenyl, heterocycle, C₂-C₈ alkenyl, C₂-C₈ alkynyl, C₁-C₈ alkoxy, C₁-C₈ acyl, C₁-C₈ acyloxy, phenoxy, heteroaryloxy and C₁-C₈ alkylthio may be substitited by halogen atom, heterocycle, C₁-C₈ alkoxy, C₁-C₈ acyl, C₁-C₄ alkylsulfonyl, cyano, C₁-C₄ alkoxycarbonyl, hydroxyl, C₁-C₈ acyloxy, heteroaryloxy, C₁-C₈ alkylthio, phenyl, phenoxy or amino, or Z¹ and Z² may be combined to represent divalent atomic group —O—CH₂—O—.

5. The keratinocyte-proliferation suppresor according to claim 1 or 2, comprising as active ingredient a compound of the general formula (Ic): 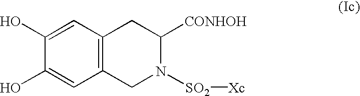
or a pharmaceutically acceptable salt thereof, wherein
Xc is atomic group of the following formula (H), (I), (J) or (K): 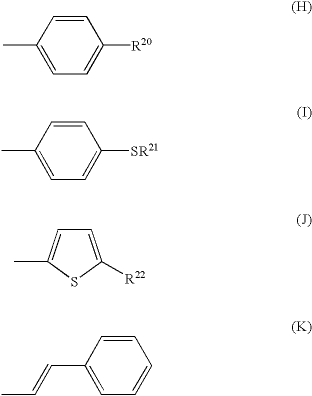
wherein R²⁰ is vinyl, ethynyl, pyridyloxy, pyrazyloxy, C₁-C₈alkyl, C₁-C₈ acyl or C₁-C₈ alkoxy, wherein the said C₁-C₈ alkyl, C₁-C₈ acyl or C₁-C₈ alkoxy may be substituted by phenyl, halogen atom, C₁-C₄ alkoxy, C₁-C₄ alkylthio, C₁-C₄ alkylsulfonyl, cyano, C₁-C₄ alkoxycarbonyl, C₁-C₄ acyloxyorhydroxyl; R²¹ and R²² are hydrogen atom or C₁-C₈alkyl, wherein the said C₁-C₈ alkyl may be substituted by C₁-C₄ alkoxy.

6. A compound of the general formula (T): 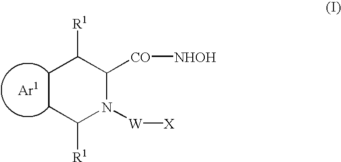
or a pharmaceutically acceptable salt thereof, wherein
Ar¹ is any cyclic structure selected from the following formulas (A) to (C): 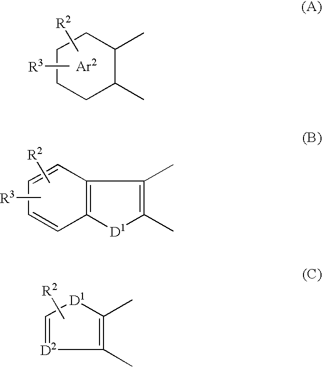
wherein Ar² is 6-membered aromatic ring which may include 1 to 2 nitrogen atoms, D¹ is —(CH₂)ₙ— (n is integer of 1 to 3), oxygen atom, sulfur atom, —S(O)—, —S(O)₂— or —NR⁴— (R⁴ is hydrogen atom, C₁-C₈ alkyl or C₁-C₈ acyl), D² is —(CH)— or nitrogen atom, R² and R³ may be the same or different, and are hydrogen atom, hydroxyl, C₁-C₈ alkyl, C₂-C₈ alkenyl, C₂-C₈ alkynyl, halogen atom, —NR⁵R⁶ (R⁵ and R⁶ may be the same or different, and are hydrogen atom, C₁-C₈ alkyl, C₁-C₈ acyl, carbamoyl or C₁-C₄ alkylcarbamoyl), wherein the said C₁-C₈ alkyl, C₂-C₈ alkenyl, C₂-C₈ alkynyl may be substituted by halogen atom, heterocycle, C₁-C₄ alkoxy, C₁-C₄ acyl, C₁-C₄ acyloxy, heteroaryloxy, C₁-C₄ alkylthio, phenyl, phenoxy or NR⁷R⁸— (R⁷ and R8 may be the same or different, and are hydrogen atom, C₁-C₈ alkyl or C₁-C₈ acyl); R¹ is hydrogen atom or methyl; W is divalent atomic group of the following formula (IIa) or (IIb): 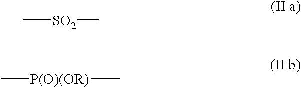
R is hydrogen atom, C₁-C₈ alkyl or phenyl, wherein the said C₁-C₈ alkyl and phenyl may be substituted by halogen atom, hydroxyl, nitrile, C₁-C₄ alkyloxycarbonyl, carboxyl, carbamoyl, C₁-C₄ alkylcarbamoyl, C₂-C₄alkenyl, C₂-C₄alkynyl, heterocycle, C₁-C₄ alkoxy, C₁-C₄acyl, C₁-C₄acyloxy, heteroaryloxy, C₁-C₄alkylthio, phenyl, biphenyl, phenoxy, C₁-C₄ alkylsulfonyl, arylsulfonyl, arylaminosulfonyl or NR⁹R¹⁰ (R⁹ and R¹⁰maybe the same or different, and are hydrogen atom, C₁-C₈ alkyl, C₁-C₈ acyl, carbamoyl or C₁-C₄ alkylcarbamoyl); X is atomic group of the following formula: 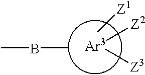
wherein cyclic structure Ar³ is benzene ring or 5- or 6-membered heteroaromatic ring including one oxygen atom, one sulfur atom or 1 to 2 nitrogen atoms, B is covalent bond, —(CH2)_{q}— (q is integer of 1 to 4), —CH═CH—, —C(O)—, —CH(OH)— or —NR¹¹—, R¹¹ is hydrogen atom, C₁-C₈ alkyl, C₂-C₈ alkenyl or C₂-C₈ alkynyl, wherein the said C₁-C₈ alkyl, C₂-C₈ alkenyl and C₂-C₈ alkynyl may be substituted by halogen atom, heterocycle, C₁-C₈ alkoxy, C₁-C₈ acyl, C₁-C₈ acyloxy, heteroaryloxy, C₁-C₈ alkylthio, phenyl, phenoxy or —NR¹²R¹³ (R12 and R13 may be the same or different, and are hydrogen atom, C₁-C₈ alkyl, C₁-C₈ acyl, carbamoyl or C₁-C₄ alkylcarbamoyl); Z¹, Z² and Z³may be the same or different, and are hydrogen atom, C₁-C₈ alkyl, C₂-C₈ alkenyl, C₂-C₈ alkynyl, phenyl, heterocycle, halogen atom, hydroxyl, C₁-C₈alkoxy, C₁-C₈ acyl, C₁-C₈ acyloxy, phenoxy, heteroaryloxy, C₁-C₈ alkylthio or —NR¹⁴R⁵ (R⁴ and R¹⁵ may be the same or different, and are hydrogen atom, C₁-C₈ alkyl, C₁-C₈ acyl, carbamoyl or C₁-C₄ alkylcarbamoyl), wherein the said C₁-C₈ alkyl, phenyl, heterocycle, C₂-C₈alkenyl, C₂-C₈ alkynyl, C₁-C₈ alkoxy, C₁-C₈ acyl, C₁-C₈ acyloxy, phenoxy, heteroaryloxy and C₁-C₈ alkylthio may be substituted by halogen atom, heterocycle, C₁-C₈ alkoxy, C₁-C₈ acyl, C₁-C₄ alkylsulfonyl, cyano, C₁-C₄ alkoxycarbonyl, hydroxyl, C₁-C₈ acyloxy, heteroaryloxy, C₁-C₈ alkylthio, phenyl, phenoxy or amino, or Z¹ and Z² may be combined to represent divalent atomic group —O—CH₂—O—.

7. A compound of the general formula (Ia): 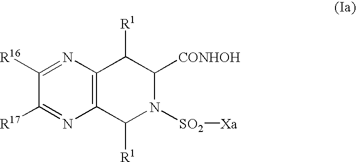
or a pharmaceutically acceptable salt thereof, wherein
R¹ is hydrogen atom or methyl, R and R¹⁷ may be the same or different, and are hydrogen atom or C₁-C₄ alkyl, Xa is atomic group of the following formula (D), (E), (F) or (G): 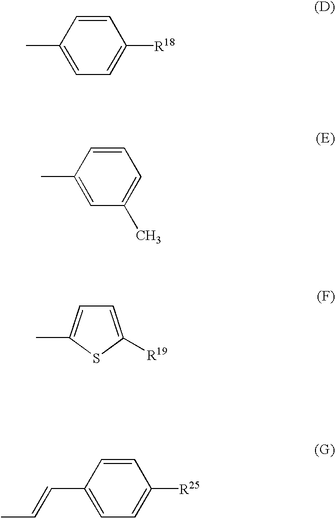
2-propynyl, 2-butynyl, phenyl, halogen, C₁-C₄ alkoxy, C₁-C₄ alkylthio, C₁-C₄ alkylsulfonyl, cyano, C₁-C₄ alkoxycarbonyl, C₁-C₄ acyloxy or hydroxyl; R¹⁹ is hydrogen atom or C₁-C₈ alkyl, wherein the said C₁-C₈ alkyl may be substituted by C₁-C₄ alkoxy, R²⁵ is hydrogen atom or C₁-C₈ alkoxy).

8. A compound of the general formula (Ib): 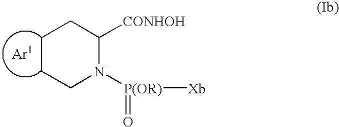
or a pharmaceutically acceptable salt thereof, wherein
Ar¹ is any cyclic structure selected from the following formulas (A) to (C): 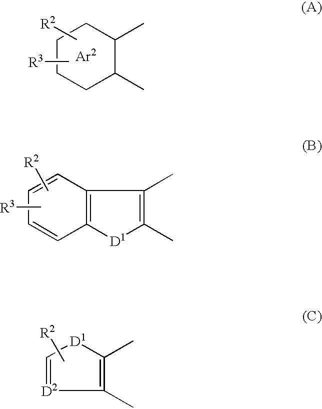
wherein Ar is 6-membered aromatic ring which may include 1 to 2 nitrogen atoms, D¹ is —(CH₂)ₙ— (n is integer of 1 to 3), oxygen atom, sulfur atom, —S(O)—, —S(O)₂— or NR⁴— (R⁴ is hydrogen atom, C₁-C₈ alkyl or C₁-C₈ acyl), D² is —(CH)— or nitrogen atom, R² and R³may be the same or different, and are hydrogen atom, hydroxyl, C₁-C₈ alkyl, C₂-C₈ alkenyl, C₂-C₈ alkynyl, halogen atom, —NR⁵R⁶ (R⁵ and R⁶ may be the same or different, and are hydrogen atom, C₁-C₈ alkyl, C₁-C₈ acyl, carbamoyl or C₁-C₄ alkylcarbamoyl), wherein the said C₁-C₈ alkyl, C₂-C₈ alkenyl, C₂-C₈ alkynyl may be substituted by halogen atom, heterocycle, C₁-C₄ alkoxy, C₁-C₄ acyl, C₁-C₄ acyloxy, heteroaryloxy, C₁-C₄ alkylthio, phenyl, phenoxy or —NR⁷R⁸ (R⁷ and R⁸ may be the same or different, and are hydrogen atom, C₁-C₈ alkyl or C₁-C₈ acyl);
R is hydrogen atom, C₁-C₈ alkyl or phenyl, wherein the said C₁-C₈ alkyl and phenyl may be substituted by halogen atom, hydroxyl, nitrile, C₁-C₄alkyloxycarbonyl, carboxyl, carbamoyl, C₁-C₄ alkylcarbamoyl, C₂-C₄ alkenyl, C₂-C₄ alkynyl, heterocycle, C₁-C₄ alkoxy, C₁-C₄ acyl, C₁-C₄ acyloxy, heteroaryloxy, C₁-C₄ alkylthio, phenyl, biphenyl, phenoxy, C₁-C₄ alkylsulfonyl, arylsulfonyl, arylaminosulfonyl or —NR⁹R¹⁰ (R⁹ and R¹⁰ may be the same or different, and are hydrogen atom, C₁-C₈ alkyl, C₁-C₈ acyl, carbamoyl or C₁-C₄ alkylcarbamoyl); Xb is atomic group of the following formula: 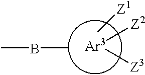
wherein cyclic structure Ar³ is benzene ring or 5- or 6-membered heteroaromatic ring including one oxygen atom, one sulfur atom or 1 to 2 nitrogen atoms, B is covalent bond, —(CH2)_{q}— (q is integer of 1 to 4), —CH═CH—, —C(O)—, CH(OH)— or NR¹¹—, R¹¹ is hydrogenatom, C₁-C₈alkyl, C₂-C₈alkenylor C₂-C₈alkynyl, wherein the said C₁-C₈ alkyl, C₂-C₈ alkenyl and C₂-C₈ alkynyl may be substituted by halogen atom, heterocycle, C₁-C₈ alkoxy, C₁-C₈ acyl, C₁-C₈ acyloxy, heteroaryloxy, C₁-C₈ alkylthio, phenyl, phenoxy or NR¹²R¹³ (R¹² and R¹³ may be the same or different, and are hydrogen atom, C₁-C₈ alkyl, C₁-C₈ acyl, carbamoyl or C₁-C₄ alkylcarbamoyl); Z¹, Z² and Z³ may be the same or different, and are hydrogen atom, C₁-C₈ alkyl, C₂-C₈ alkenyl, C₂-C₈ alkynyl, phenyl, heterocycle, halogenatom, hydroxyl, C₁-C₈alkoxy, C₁-C₈ acyl, C₁-C₈ acyloxy, phenoxy, heteroaryloxy, C₁-C₈ alkylthio or —NR¹⁴R¹⁵ (R¹⁴ and R¹⁵ may be the same or different, and are hydrogen atom, C₁-C₈ alkyl, C₁-C₈ acyl, carbamoyl or C₁-C₄ alkylcarbamoyl), wherein the said C₁-C₈ alkyl, phenyl, heterocycle, C₂-C₈ alkenyl, C₂-C₈ alkynyl, C₁-C₈ alkoxy, C₁-C₈ acyl, C₁-C₈ acyloxy, phenoxy, heteroaryloxy and C₁-C₈ alkylthio may be substituted by halogen atom, heterocycle, C₁-C₈ alkoxy, C₁-C₈ acyl, C₁-C₄ alkylsulfonyl, cyano, C₁-C₄ alkoxycarbonyl, hydroxyl, C₁-C₈ acyloxy, heteroaryloxy, C₁-C₈ alkylthio, phenyl, phenoxy or amino, or Z¹ and Z² may be combined to represent divalent atomic group-O—CH₂—O—.

9. A compound of the general formula (Ic): 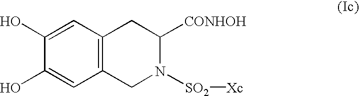
or a pharmaceutically acceptable salt thereof, wherein
Xc is atomic group of the following formula (H), (I), (J) or (K): 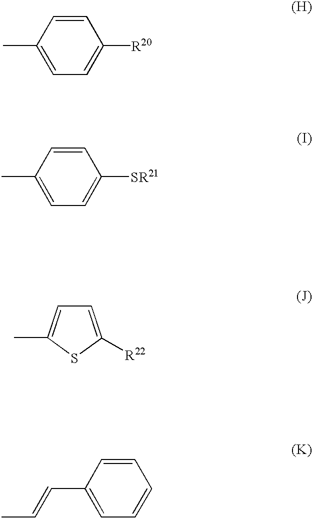
wherein R²⁰ is vinyl, ethynyl, pyridyloxy, pyrazyloxy, C₁-C₈ alkyl, C₁-C₈ acyl or C₁-C₈ alkoxy, wherein the said C₁-C₈ alkyl, C₁-C₈ acyl or C₁-C₈ alkoxy may be substituted by phenyl, halogen atom, C₁-C₄ alkoxy, C₁-C₄ alkylthio, C₁-C₄ alkylsulfonyl, cyano, C₁-C₄ alkoxycarbonyl, C₁-C₄acyloxyorhydroxyl; R²¹ and R²² are hydrogen atom or C₁-C₈alkyl, wherein the said C₁-C₈alkyl may be substituted by C₁-C₄ alkoxy.
